(19) United States
(12) Patent Application Publication
(10) Pub. No.: US 2010/0022620 A1
(43) Pub. Date: Jan. 28, 2010

(54) EPITOPE REDUCTION THERAPY

(76) Inventors: Matthew David Max Crispin, Oxford (GB); Christopher Scanlan, Oxford (GB); Frances Mary Platt, Oxford (GB); Hugh John Willison, Glasgow (GB)

Correspondence Address: NIXON & VANDERHYE, PC, 901 NORTH GLEBE ROAD, 11TH FLOOR, ARLINGTON, VA, 22203, US

(21) Appl. No.: 12/375,068

(22) PCT Filed: Jul. 27, 2007
(86) PCT No.: PCT/GB2007/002861

§ 371 Date: Oct. 5, 2009

(30) Foreign Application Priority Data
Jul. 27, 2006 (GB) 0614947.0

(51) Int. Cl. A61K 31/7088; A61K 31/70; A61P 25/28; A61P 3/10

(52) U.S. Cl. 514/44 A; 514/44 R; 514/43

(57) ABSTRACT

The present invention provides the use of an inhibitor of glycolipid biosynthesis in the manufacture of a medicament for the treatment of a glycolipid-mediated autoimmune disease.

### FIELD OF THE INVENTION

The present invention relates to the treatment of glycolipid-mediated autoimmnune diseases.

### BACKGROUND TO THE INVENTION

Anti-glycolipid antibodies can mediate tissue damage and destruction. Anti-glycolipid antibodies, such as anti-glycolipid autoantibodies, are found in a range of diseases including: Guillain-Barré syndrome; variants of Guillain-Barré syndrome; Guillain-Barré syndrome with opthalmoplegia; Miller Fisher syndrome; cranial nerve variants of Miller Fischer syndrome, for instance Bickerstaff's brainstem encephalitis; Acute motor axonal neuropathy; Motor neuropathy; Motor neuropathy with multifocal conduction blocks; Lower motor neuron syndromes; Chronic inflammatory demyelinating polyneuropathy; Multifocal chronic inflammatory demyelinating polyneuropathy; Acute inflammatory demyelinating polyneuropathy; Subacute inflammatory demyelinating polyneuropathy; Sensory neuropathies; Multifocal Motor Neuropathy; Multifocal motor sensory neuropathy; Acute Motor Sensory Axonal neuropathy; Multifocal motor demyelinating neuropathy; Chronic idiopathic sensory ataxic neuropathy; Chronic recurrent polyneuropathy; Mixed motor sensory neuropathy; Sciatica; Autoimmune mononeuritis multiplex; Acute relapsing sensory-dominant polyneuropathy associated with anti-GQ1b antibody; Amyotrophic lateral sclerosis; Diabetic neuropathy; Acute panautonomic neuropathy; Bell's palsy; Acute opthalmoparesis; Multiple sclerosis; Transverse myelitis; Optic neuritis; Chronic myelinic neuropathy with IgM gammopathy; Cryptogenic partial epilepsies; Partial oculomotor nerve palsy; Isolated cranial neuropathy; Autoimmune cerebellar disease; Acute Disseminated Encephalomyelitis; Stiff-man syndrome; Bickerstaff's brainstem encephalopathy; Systemic lupus erythamatosus; Discoid lupus; Scleroderma; Morphoea; CREST; Mixed connective tissue disease; Relapsing polychondritis; Sjogren's syndrome; Primary fibromyalgia syndrome; an autoimmune complication of drug therapy with Tumor necrosis factor-α blocker, Interferon-α, Tacrolimus (FK506), Cyclosporine A, Suramin, Zimeldine, Cisplatin, Captopril, Danazol, Gold, Penicillamine, Streptokinase or Anistreplase; an autoimmune complication of vaccination with Influenza Vaccination; an autoimmune complication of vaccination with Menactra meningococcal conjugate vaccine; Fibromyalgia syndrome; Chronic fatigue syndrome; Behçet's disease; Hashimoto's thyroiditis; Graves' disease; Alzheimer's disease; Insulin-dependent (type I) diabetes mellitus; Neuroborreliosis; Acute Disseminated Encephalomyelitis; Guillain-Barré disease; Rheumatoid arthritis; Still's disease; Coeliac disease; Crohn's disease; Ulcerative colitis; Primary adrenal failure; Pernicious anoemia; Idiopathic thrombocytopenic purpura; IgA Nephropathy; Meniere's disease; Autoimmune hemolytic anemia; Autoimmune hepatitis; Autoimmune inner ear disease or Acute ophthahnoparesis.

In such autoimmune diseases, antibodies specific to glycolipids bind the carbohydrate moiety of the glycolipid antigen. These antibodies include IgM and differentiated class-switched antibodies. These antibodies can show considerable discrimination between the different carbohydrate structures found in different glycolipids. Glycolipids are found on all tissues and show considerable tissue specific diversity. The specificity of the antibody to a particular glycolipid antigen may influence the tissue recognised and hence the type of pathology observed. For example, in Guillain-Barré syndrome, antibody reactivity towards glycolipids such as GM1 and GD1a can lead to the development of neuropathy. Thus the localization of the autoantigen (such as GM1 and GD1a) correlates with the localisation of antibody mediated tissue damage.

Glycolipids can also be recognised by T-cells when presented in complex with CD1 molecules. Thus, autoreactive T-cells can recognise self glycolipids in autoimmune conditions. For example, T-cells from patients with multiple sclerosis show reactivity to sulfatide glycolipids in complex with CD1a.

Some glycolipid-mediated autoimmune diseases may be treated by reducing the serum levels of the destructive anti-glycolipid antibodies using plasma exchange (Harel M, et al. Clin Rev Allergy Immunol. 2005 December; 29(3):281-7), Hughes R A et al. Lancet. 2005 Nov. 5; 366(9497):1653-66). However, this is a transient ameliorative therapy which fails to remove the underlying basis of the pathology. This is reflected by the modest and/or transient effect of this therapy in most patients. In addition, plasma exchange is time and technology intensive, and is not practicable in many cases, for example, in countries with less developed health care facilities. A recent potential development over plasma exchange is the use of glycolipid resins to adsorb glycolipid reactive autoantibodies from patient sera. Again, however, this approach suffers from the limitation that it does not alter the antigenic stimulus that maintains pathology. A further method for the treatment of glycolipid-mediated autoimmune diseases such as Guillain-Barré syndrome is intravenous immunoglobulin (IVIg) treatment. However, IVIg treatment has limited efficacy with side effects ranging from anaphylactic reactions to serum sickness-type symptoms. There is therefore a need to develop improved treatments for glycolipid-mediated autoimmune diseases.

### SUMMARY OF THE INVENTION

Immunologically “self” epitopes (i.e. autoantigens) are recognised by autoreactive antibodies and T-cells, leading to immune pathology. The present invention relates to the removal or reduction of self-antigens as a direct and targeted approach to treating autoimmmunity. It is believed that the abundance of many self-epitopes can be controlled by metabolic or pharmaceutical intervention without serious unwanted effects, and consequently that autoimmunity can be reduced or eliminated by inhibiting the synthesis or expression of endogenous self antigens. Specifically, this applies to the synthesis or expression of glycolipid antigens which are associated with a range of clinically distinct pathologies in which antibody or T-cell mediated immunity to the glycolipids leads to disease. It is believed that inhibition of glycolipid synthesis will reduce epitope formation and hence reduce anti-glycolipid mediated tissue damage.

Accordingly, the present invention provides the use of an inhibitor of glycolipid biosynthesis in the manufacture of a medicament for the treatment of a glycolipid-mediated autoimmune disease.

The invention also provides a method of treating a glycolipid-mediated autoimmune disease, which method comprises administering to a patient in need of such treatment an effective amount of an inhibitor of glycolipid biosynthesis.

The invention also provides a pharmaceutical composition for use in treating a glycolipid-mediated autoimmune disease, comprising a pharmaceutically acceptable carrier or diluent and an inhibitor of glycolipid biosynthesis.

The invention also provides an inhibitor of glycolipid biosynthesis for use in treating a glycolipid-mediated autoimmune disease.

The invention also provides an agent for the treatment of a glycolipid-mediated autoimmune disease, comprising an inhibitor of glycolipid biosynthesis.

Typically, the inhibitor of glycolipid biosynthesis is a compound of the following formula (I), formula (II), formula (III), formula (IV), formula (V), formula (IX) or formula (XII):

Accordingly, the invention further provides the use, in the manufacture of a medicament for the treatment of a glycolipid-mediated autoimmune disease, of a compound of formula (I), formula (II), formula (III), formula (IV), formula (V), formula (IX) or formula (XII):

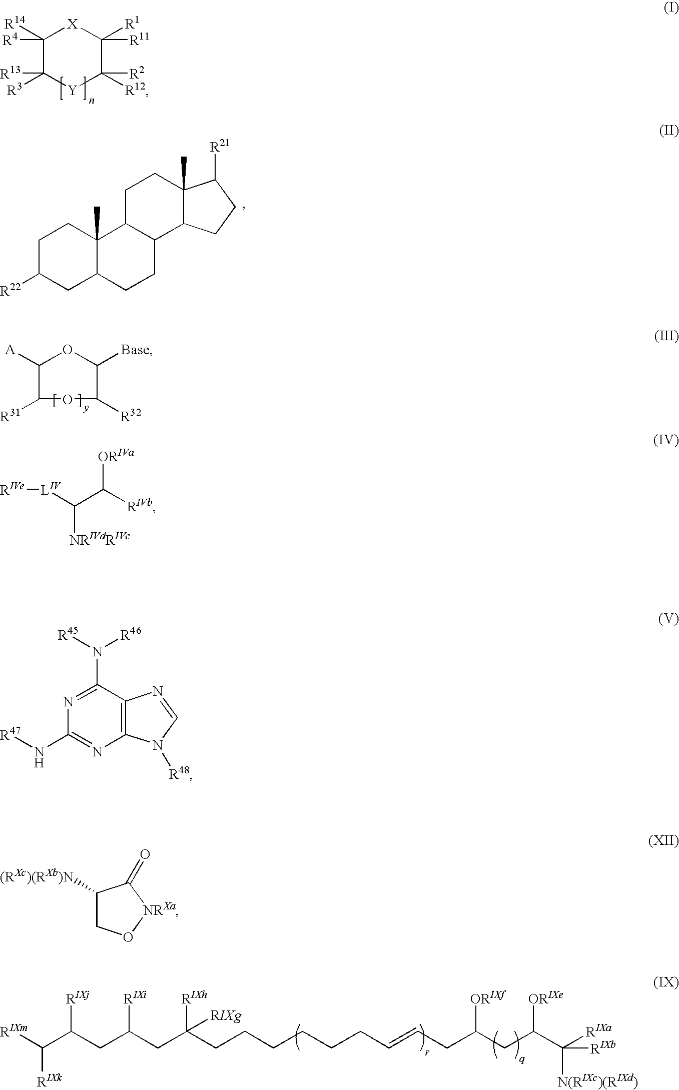

wherein:

X is O, S or NR⁵;

R⁵ is hydrogen, substituted or unsubstituted C₁₋₂₀ alkyl, substituted or unsubstituted C₁₋₂₀ alkylene-aryl, substituted or unsubstituted C₁₋₂₀ alkylene-C₃₋₂₀ heteroaryl, substituted or unsubstituted C₁₋₂₀ alkylene-C₃₋₂₅ cycloalkyl, substituted or unsubstituted C₁₋₂₀ alkylene-C₃₋₂₀ heterocyclyl, substituted or unsubstituted C₁₋₂₀ alkylene-O—C₃₋₂₀ heterocyclyl, substituted or unsubstituted aryl, substituted or unsubstituted C₃₋₂₀ heteroaryl, substituted or unsubstituted C₃₋₂₅ cycloalkyl or substituted or unsubstituted C₃₋₂₀ heterocyclyl, or R⁵ forms, together with R¹, R¹¹, R⁴ or R¹⁴, a substituted or unsubstituted C₁₋₆ alkylene group, wherein said C₁₋₂₀ alkyl and C₁₋₂₀ alkylene are optionally interrupted by N(R′), O, S or arylene wherein R′ is H, C₁₋₆ alkyl or aryl;

n is 0 or 1;

Y is O, S or CR⁶R⁶;

R¹, R¹¹, R⁴ and R¹⁴, which may be the same or different, are independently selected from hydrogen, hydroxyl, carboxyl, amino, thiol, substituted or unsubstituted C₁₋₂₀ alkyl, substituted or unsubstituted C₁₋₂₀ alkoxy, substituted or unsubstituted aryloxy, acyl, ester, acyloxy, C₁₋₁₀ alkylamino, di(C₁₋₁₀)alkylamino, amido, acylamido, —O—C₃₋₂₅ cycloalkyl and —O—C₃₋₂₀ heterocyclyl, provided that one of R¹, R¹¹, R⁴ and R¹⁴ may form, together with R⁵, a substituted or unsubstituted C₁₋₆ alkylene group, wherein said C₁₋₂₀ alkyl is optionally interrupted by N(R′), O, S or arylene;

R², R¹², R³, R¹³, R⁶ and R¹⁶, which may be the same or different, are independently selected from hydrogen, hydroxyl, carboxyl, amino, thiol, substituted or unsubstituted C₁₋₂₀ alkyl, substituted or unsubstituted C₁₋₂₀ alkoxy, substituted or unsubstituted aryloxy, acyl, ester, acyloxy, C₁₋₁₀ alkylamino, di(C₁₋₁₀)alkylamino, amido, acylamido —O—C₃₋₂₅ cycloalkyl and —O—C₃₋₂₀ heterocyclyl, wherein said C₁₋₂₀ alkyl is optionally interrupted by N(R′), O, S or arylene;

R²¹ is selected from oxo, -L³⁰-R²³, -L³⁰-C(O)N(H)—R²⁴ and a group of the following formula (VI):

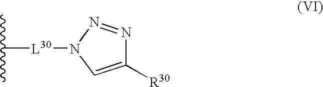

L³⁰ is substituted or unsubstituted C₁₋₂₀ alkylene which is optionally interrupted by N(R′), O, S or arylene;

R²³ is carboxyl, hydroxyl, ester, phosphonate ester, phosphate ester, phosphoric acid and phosphonic acid;

R²⁴ is C₁₋₂₀ alkyl which is unsubstituted or substituted with one or more groups selected from carboxyl, hydroxyl, ester, phosphonate ester, phosphate ester, phosphoric acid and phosphonic acid, wherein said C₁₋₂₀ alkyl is optionally interrupted by N(R′), O, S or arylene;

R³⁰ is C₁₋₂₀ alkyl which is unsubstituted or substituted with one or more groups selected from carboxyl, hydroxyl, ester, amino, phosphonate ester, phosphate ester, phosphoric acid and phosphonic acid, wherein said C₁₋₂₀ alkyl is optionally interrupted by N(R′), O, S or arylene;

R²² is hydroxyl, oxo, acyloxy, phosphoric acid or —OC(O)-alk-C(O)OH, wherein alk is substituted or unsubstituted C₁₋₂₀ alkylene which is optionally interrupted by N(R′), O, S or arylene;

Base is selected from a group of any one of the following formulae (a), (b), (c), (d), (e), (f) and (g):

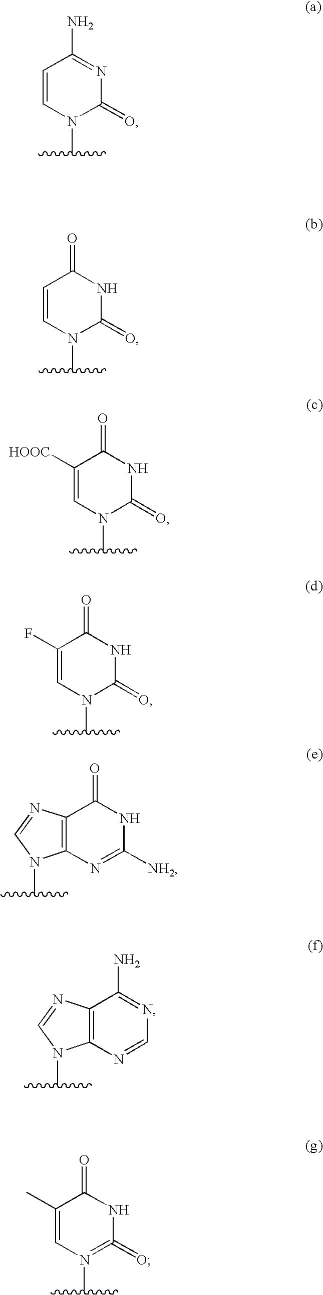

y is 0 or 1;

R³¹ is OH; R³² is H or OH; or, provided that y is 0, R³¹ and R³² together form —O—C(R³³)(R³⁴)—O—, wherein R³³ and R³⁴ are independently selected from H and methyl;

A is substituted or unsubstituted C₁₋₂₀ alkyl, substituted or unsubstituted C₁₋₂₀ alkylene-aryl, substituted or unsubstituted C₁₋₂₀ alkylene-C₃₋₂₀ heteroaryl, substituted or unsubstituted C₁₋₂₀ alkylene-C₃₋₂₅ cycloalkyl or substituted or unsubstituted C₁₋₂₀ alkylene-C₃₋₂₀ heterocyclyl, wherein said C₁₋₂₀ alkyl and C₁₋₂₀ alkylene are optionally interrupted by N(R′), O, S or arylene, wherein R′ is H, C₁₋₆ alkyl or aryl, or A is a group of any one of the following formulae (g) to (k):

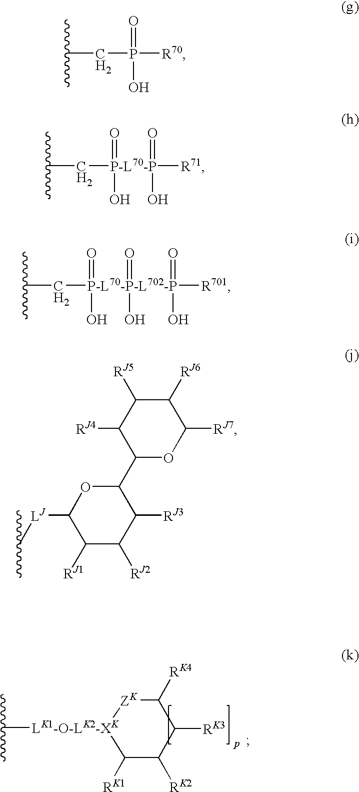

L⁷⁰, L⁷⁰¹ and L⁷⁰² are independently selected from —O—, —C(R³⁵)(R³⁶)— and —NH—, wherein R³⁵ and R³⁶ are independently selected from H, OH and CH₃;

R⁷⁰, R⁷¹ and R⁷⁰¹ are selected from OH, substituted or unsubstituted C₁₋₂₀ alkyl, substituted or unsubstituted C₁₋₂₀ alkoxy, substituted or unsubstituted C₁₋₁₀ alkylamino and -L⁷¹-(X²)ₘ-L⁷²-R⁷²; wherein m is 0 or 1; X² is O, S, —C(R⁴⁵)(R⁴⁶)— or —O—C(R⁴⁵)(R⁴⁶)—, wherein R⁴⁵ and R⁴⁶ are independently selected from H, OH, phosphonic acid or a phosphonic acid salt; L⁷¹ and L⁷² are independently selected from a single bond and substituted or unsubstituted C₁₋₂₀ alkylene, which C₁₋₂₀ alkylene is optionally interrupted by N(R′), O, S or arylene, wherein R′ is H, C₁₋₆ alkyl or aryl; and R⁷² is C₃₋₂₅ cycloalkyl or C₃₋₂₀ heterocyclyl;

L^{J} is substituted or unsubstituted C₁₋₂₀ alkylene;

R^{J1}, R^{J2}, R^{J3}, R^{J4}, R^{J5}, R^{J6} and R^{J7}, which are the same or different, are independently selected from hydrogen, hydroxyl, carboxyl, amino, thiol, substituted or unsubstituted C₁₋₂₀ alkyl, substituted or unsubstituted C₁₋₂₀ alkoxy, substituted or unsubstituted aryloxy, acyl, ester, acyloxy, C₁₋₁₀ alkylamino, di(C₁₋₁₀)alkylamino, amido, acylamido, —N(H)C(O)CH═CH—R^{3e}, —O—C₃₋₂₅ cycloalkyl and —O—C₃₋₂₀ heterocyclyl, wherein said C₁₋₂₀ alkyl is optionally interrupted by NR′), O, S or arylene, and wherein R^{J8} is substituted or unsubstituted C₁₋₂₀ alkyl;

L^{K1} and L^{K2}, which are the same or different, are independently selected from a single bond and substituted or unsubstituted C₁₋₂₀ alkylene;

X^{K} is N or C(R^{K6}), wherein R^{K6} is H, COOH or ester;

Z^{K} is O or CH(R^{K5});

p is 0 or 1;

R^{K1}, R^{K2}, R^{K3}, R^{K4} and R^{K5}, which are the same or different, are independently selected from hydrogen, hydroxyl, carboxyl, amino, thiol, substituted or unsubstituted C₁₋₂₀ alkyl, substituted or unsubstituted C₁₋₂₀ alkoxy, substituted or unsubstituted aryloxy, acyl, ester, acyloxy, C₁₋₁₀ alkylamino, di(C₁₋₁₀)alkylamino, amido, acylamido, —O—C₃₋₂₅ cycloalkyl and —O—C₃₋₂₀ heterocyclyl, wherein said C₁₋₂₀ alkyl is optionally interrupted by N(R′), O, S or arylene;

R^{IVa} and R^{IVd}, which are the same or different, are independently selected from H, substituted or unsubstituted C₁₋₆ alkyl or substituted or unsubstituted phenyl;

R^{IVb} is H, substituted or unsubstituted aryl, —CH═CHR^{IVf}, or substituted or unsubstituted C₁₋₂₀ alkyl, which C₁₋₂₀ alkyl is optionally interrupted by N(R′), O, S or arylene wherein R′ is H, C₁₋₆ alkyl or aryl;

R^{IVc} is H, substituted or unsubstituted C₁₋₆ alkyl, substituted or unsubstituted phenyl or —C(O)R^{IVg};

R^{IVf} is H or substituted or unsubstituted C₁₋₂₀ alkyl, which C₁₋₂₀ alkyl is optionally interrupted by N(R′), O, S or arylene;

R^{IVg} is H or substituted or unsubstituted C₁₋₂₀ alkyl, which C₁₋₂₀ alkyl is optionally interrupted by N(R′), O, S or arylene;

R^{IVe} is H, hydroxyl, carboxyl, amino, thiol, substituted or unsubstituted C₁₋₂₀ alkyl, substituted or unsubstituted C₁₋₂₀ alkoxy, substituted or unsubstituted aryloxy, acyl, ester, acyloxy, C₁₋₁₀ alkylamino, di(C₁₋₁₀)alkylamino, amido, acylamido, —O—C₃₋₂₅ cycloalkyl, —O—C₃₋₂₀ heterocyclyl, substituted or unsubstituted aryl, substituted or unsubstituted C₃₋₂₀ heteroaryl, substituted or unsubstituted C₃₋₂₅ cycloalkyl or substituted or unsubstituted C₃₋₂₀ heterocyclyl, which C₁₋₂₀ alkyl is optionally interrupted by N(R′), O, S or arylene;

L^{IV} is substituted or unsubstituted C₁₋₂₀ alkylene which C₁₋₂₀ alkylene is optionally interrupted by N(R′), O, S or arylene;

R⁹¹ and R⁹², which are the same or different, are independently selected from H, substituted or unsubstituted C₁₋₂₀ alkyl, substituted or unsubstituted aryl and -L⁹¹-R⁹⁵, wherein L⁹¹ is substituted or unsubstituted C₁₋₂₀ alkylene, wherein said C₁₋₂₀ alkyl and said C₁₋₂₀ alkylene are optionally interrupted by N(R′), O, S or arylene wherein R′ is H, C₁₋₆ alkyl or aryl, and wherein R⁹⁵ is substituted or unsubstituted aryl, amino, C₁₋₁₀ alkylamino or di(C₁₋₁₀)alkylamino;

R⁹³ is -L⁹²-R⁹⁶, wherein L⁹² is a single bond or substituted or unsubstituted C₁₋₂₀ alkylene, which C₁₋₂₀ alkylene is optionally interrupted by N(R′), O, S or arylene, and wherein R⁹⁶ is amido or substituted or unsubstituted aryl;

R⁹⁴ is H or substituted or unsubstituted C₁₋₂₀ alkyl, which C₁₋₂₀ alkyl is optionally interrupted by N(R′), O, S or arylene;

q is 0 or 1;

r is 0 or 1;

R^{IXa} is H, COOH or an unsubstituted or substituted ester;

R^{IXb} is an unsubstituted or substituted C₁₋₆ alkyl;

R^{IXc} and R^{IXd}, which are the same or different, are each independently selected from H, unsubstituted or substituted C₁₋₆ alkyl and unsubstituted or substituted phenyl;

R^{IXe} and R^{IXf}, which are the sane or different, are each independently selected from H, unsubstituted or substituted C₁₋₆ alkyl, unsubstituted or substituted phenyl and unsubstituted or substituted acyl;

either (a) one of R^{IXg} and R^{IXh} is H and the other is OR^{IXr}, wherein R^{IXr} is selected from H, unsubstituted or substituted C₁₋₆ alkyl, unsubstituted or substituted phenyl and unsubstituted or substituted acyl, or (b) R^{IXg} and R^{IXh} together form an oxo group;

R^{IXi} is H, unsubstituted or substituted C₁₋₆ alkyl, unsubstituted or substituted C₁₋₆ alkoxy and unsubstituted or substituted phenyl;

R^{IXj} is H, unsubstituted or substituted C₁₋₆ alkyl or a group of the following formula (X):

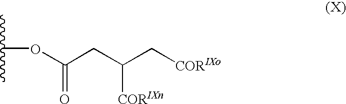

in which R^{IXn} and R^{IXo}, which are the same or different, are each independently selected from OH, unsubstituted or substituted C₁₋₆ alkoxy, unsubstituted or substituted phenoxy, amino, unsubstituted or substituted C₁₋₆ alkylamino and unsubstituted or substituted di(C₁₋₆)alkylamino;

R^{IXk} is H, unsubstituted or substituted C₁₋₆ alkyl or a group of the following formula (XI):

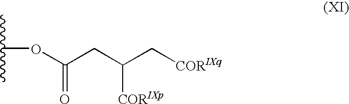

in which R^{IXp} and R^{IXq}, which are the same or different, are each independently selected from OH, unsubstituted or substituted C₁₋₆ alkoxy, unsubstituted or substituted phenoxy, amino, unsubstituted or substituted C₁₋₆ alkylamino and unsubstituted or substituted di(C₁₋₆)alkylamino;

R^{IXm} is selected from H and unsubstituted or substituted C₁₋₂₀ alkyl, which C₁₋₂₀ alkyl is optionally interrupted by N(R′), O, S or phenylene, wherein R′ is H, C₁₋₆ alkyl or phenyl;

R^{Xa} is H, substituted or unsubstituted C₁₋₂₀ alkyl, substituted or unsubstituted C₁₋₂₀ alkylene-aryl, substituted or unsubstituted C₁₋₂₀ alkylene-C₃₋₂₀ heteroaryl, substituted or unsubstituted C₁₋₂₀ alkylene-C₃₋₂₅ cycloalkyl, substituted or unsubstituted C₁₋₂₀ alkylene-C₃₋₂₀ heterocyclyl, substituted or unsubstituted C₁₋₂₀ alkylene-O—C₃₋₂₀ heterocyclyl, substituted or unsubstituted aryl, substituted or unsubstituted C₃₋₂₀ heteroaryl, substituted or unsubstituted C₃₋₂₅ cycloalkyl or substituted or unsubstituted C₃₋₂₀ heterocyclyl wherein said C₁₋₂₀ alkyl and C₁₋₂₀ alkylene are optionally interrupted by N(R′), O, S or arylene wherein R′ is H, C₁₋₆ alkyl or aryl; and

R^{Xb} and R^{Xc}, which are the same or different, are independently selected from H, unsubstituted or substituted C₁₋₁₀ alkyl and unsubstituted or substituted aryl;

or a pharmaceutically acceptable salt thereof.

Alternatively, the inhibitor of glycolipid biosynthesis is RNA.

Accordingly, the invention further provides the use of RNA in the manufacture of a medicament for the treatment of a glycolipid-mediated autoimmune disease.

### BRIEF DESCRIPTION OF THE FIGURES

FIG. 1 contains four micrographs, (a) to (d), of which:

(a) is a light image of control-treated human neuroblastoma cells;

(b) is a light image of human neuroblastoma cells after treatment with 500 μM NB-DNJ;

(c) is an image of the distribution of fluorescent (Alexa-Fluor 488) anti-GM1 IgG in the control treated cells; and

(d) is an image of the distribution of fluorescent (Alexa-Fluor 488) anti-GM1 IgG in the NB-DNJ treated cells.

FIG. 2 contains three graphs, (a) to (c), showing the effects of (a) 1 μM, (b) 5 μM and (c) 10 μM NB-DNJ respectively on PC12 ganglioside content, measured by anti-ganglioside antibody binding. The y-axes represent mean fluorescence, in units of % of fluorescence observed at day 0. A to D on the x-axes represent, respectively, days 0, 1, 2 and 3 following exposure to NB-DNJ; E and F represent respectively 2 and 3 days post compound-wash-out.

FIG. 3 contains three graphs, (a) to (c), showing the effect of (a) 50 μM, (b) 100 M and (c) 500 μM NB-DNJ respectively on PC12 ganglioside content, measured by anti-ganglioside antibody binding. The y-axes represent mean fluorescence, in units of % of fluorescence observed at day 0. A to D on the x-axes represent, respectively, days 0, 1, 2 and 3 following exposure to NB-DNJ; E and F represent respectively 2 and 3 days post compound-wash-out.

FIG. 4 contains three graphs, (a) to (c), showing the effect of the reductions in ganglioside levels using (a) 1 μM, (b) 5 μM and (c) 10 μM NB-DNJ respectively on anti-ganglioside antibody cytotoxicity. The y-axes represent lysis observed, in units of % of lysis observed at day 0. A to D on the x-axes represent, respectively, days 0, 1, 2 and 3 following exposure to NB-DNJ; E and F represent respectively 2 and 3 days post compound-wash-out.

FIG. 5 contains three graphs, (a) to (c), showing the effect of the reductions in ganglioside levels using (a) 50 μM, (b) 100 μM and (c) 500 μM NB-DNJ respectively on anti-ganglioside antibody cytotoxicity. The y-axes represent lysis observed, in units of % of lysis observed at day 0. A to D on the x-axes represent, respectively, days 0, 1, 2 and 3 following exposure to NB-DNJ; E and F represent respectively 2 and 3 days post compound-wash-out.

FIG. 6 is a bar chart showing the levels of various GSL species, measured by HPLC, on day 3 of treatment with 1, 5, 10, 50, 100 or 500 μM NB-DNJ. The x-axis shows the HPLC retention time (GU) values of the various GSL species, and the y-axis represents the level of GSL species as a percentage of the control level. The legend indicates the NB-DNJ concentrations in units of μM.

FIG. 7 is a schematic diagram of glycosphingolipid biosynthesis, indicating the actions of the inhibitor compounds NB-DNJ; PDMP (D,L-threo-1-phenyl-2-decanoylamino-3-morpholino-1-propanol); PPMP (D,L-threo-1-phenyl-2-hexadecanoylamino-3-morpholino-1-propanol); fumonisin; myriocin; and L-cycloserine.

FIG. 8 shows a comparison of patient () and control (∘) sera binding; ELISA was carried out to compare patient and control sera antibody binding to a range of gangliosides. The p values were calculated to guage significance of the difference between levels of patient and control antibody binding. Values of under 0.1 were obtained for binding to GM1 and GQ1b. These were therefore selected for further analysis.

FIG. 9 is a graph of sera dilution (x axis) versus apparent antibody binding, A(450 nm), (y axis) showing patient and control sera GM1 binding. Curve (A) is the binding curve obtained for control 2, curve (B) is the binding curve for control 3, curve (C) is the binding curve for patient 8, curve (D) is the binding curve for patient 10, curve (E) is the binding curve for patient 13 and curve (F) is the binding curve for patient 15. ELISA was carried out using immobilised GM1 and increasing dilutions of patient and control sera. Apparent antibody binding decreased as sera dilution was increased.

FIG. 10 is a graph of sera dilution (x axis) versus apparent antibody binding, A(450 nm), (y axis) showing patient and control sera GQ1b binding. Curve (G) is the binding curve obtained for control 3, curve (H) is the binding curve for control 4, curve (I) is the binding curve for patient 8, curve (J) is the binding curve for patient 9, curve (K) is the binding curve for patient 10 and curve (L) is the binding curve for patient p11av. Patient and control sera anti-GQ1b binding activity was analysed by ELISA. Immobilised GQ1b was incubated with increasing dilutions of patient and control sera. Again, apparent antibody binding decreased as sera dilution was increased.

FIG. 11 shows pictures of three TLC plates, A, B(i) and B(ii), on which purified GM1 and GM2 were run. Plate A was stained using orcinol spray to detect any bands containing carbohydrate. Immuno-overlay was carried out on plates B(i) and B(ii), with patient ‘8’ serum for B(i) and control ‘2’ serum for B(ii). Patient sera showed sufficient anti-GM1 antibody binding for detection by TLC-immuno-overlay.

FIG. 12 shows pictures of three TLC lanes, labelled A(i), A(ii) and B(ii). Ganglioside extracted from RAW cells was run by TLC parallel to GM1 and GM2 standards. Lanes were then separated. GM1 and GM2 standards and one lane containing RAW extract were stained with orcinol. A(i) shows the GM1 and GM2 standards stained with orcinol and A(ii) shows the RAW extract stained with orcinol. Immuno-overlay with patient ‘8’ sera was carried out on the other lane containing RAW extract. B(ii) shows the RAW extract on which immuno-overlay was carried out. RAW cells contain sufficient GM1 for detection with orcinol or immuno-overlay with patient sera.

FIG. 13 shows the results of a TLC immuno-overlay experiment which reveals drug dependent decrease in GBS patient sera antibody binding to GM1. RAW cells were grown in media containing a range of NB-DNJ (ii) and NB-DGJ (iii) concentrations from 0 to 1000 μM. Gangliosides were extracted and run on TLC plates in duplicate parallel to GM1 standard (i). Immuno-overlay was carried out with patient ‘8’ serum (A) and control ‘4’ serum (B). A=patient serum; B=control serum; (i)=GM1 standard; (ii)=NB-DNJ (concentrations given in μM); and (iii)=NB-DGJ (concentrations given in μM).

FIG. 14 shows the results of a TLC immuno-overlay experiment with standardisation of amount of sample. Gangliosides were extracted from RAW cells grown in a range of NB-DNJ (ii) and NB-DGJ (iii) concentrations from 0 to 1000 μM, and run on TLC plates parallel to GM1 standards (i), as described for FIG. 13 (Example 6d). Overlays were carried out using patient ‘8’ serum (A) and control ‘4’ serum (B). The darker staining reflects slightly longer exposure time during ECL staining. A=patient serum; B=control serum; (i)=GM1 standard; (ii)=NB-DNJ (concentrations given in μM); and (iii)=NB-DGJ (concentrations given in μM).

FIG. 15 shows a TLC plate, with standardised amounts of sample, run in an identical manner to those shown in FIGS. 13 and 14 (Example 6d) but stained with Orcinol. Orcinol-staining reveals a drug dependent decrease in levels of GM1 in RAW cells. TLC was carried out as described for FIG. 14 (Example 6d). The plate was stained with orcinol to detect bands containing carbohydrate. (i)=GM1 standard; (ii)=NB-DNJ (concentrations given in μM); and (iii)=NB-DGJ (concentrations given in μM).

FIG. 16 shows a TLC of extracts from PC12 cells, grown in media containing a range of NB-DNJ and NB-DGJ concentrations, stained with orcinol. Gangliosides were extracted from the cells and run on the TLC plate, parallel to a purified GQ1b standard (i). Carbohydrate density was revealed through orcinol staining. (i)=GQ1b standard; (ii)=NB-DNJ (concentrations given in μM); and (iii)=NB-DGJ (concentrations given in μM).

FIG. 17 consists of two bar charts, (a) and (b), displaying the results of an HPLC analysis of the presence and relative abundance of gangliosides in PC12 cells. Bar chart (a) shows the relative abundance (y axis) of gangliosides GQ1b, GD1b, GD1a, GM1a and Gb3 (x axis) on PC12 cells grown in cell media containing 0M (A), 50 μM (B) and 1 mM (C) NB-DNJ. Bar chart (b) shows the relative abundance (y axis) of gangliosides GQ1b, GD1, GD1a, GM1a and Gb3 (x axis) on PC12 cells grown in cell media containing 0M (A), 50 μM (B) and 1 mM (C) NB-DGJ.

### DETAILED DESCRIPTION OF THE INVENTION

The term “inhibitor of glycolipid biosynthesis”, as used herein, means a compound that is capable of inhibiting the synthesis or expression of a glycolipid. Typically, the glycolipid is a glycosphingolipid (GSL). More typically, the glycolipid is a ganglioside. Alternatively, the glycolipid is a neutral GSL. Inhibitors of glycolipid biosynthesis are either known or readily identifiable, without undue experimentation, using known procedures.

GSLs are synthesized from ceramide by the sequential addition of monosaccharides mediated by Golgi-resident glycosyltransferases. The amount of GSL present on the cell surface is determined by the opposing actions of GSL catabolism, mediated by lysosomal glycosidases, and GSL biosynthesis (reviewed in, for example, Platt F M et al. Phil. Trans. R. Soc. Lond. B (2003) 358:947-954; Butters T D et al. Glycobiology (2005) 15:43-52). The two main classes of GSL are the neutral GSLs (lacto and globo series) and the gangliosides. Gangliosides contain sialic acid (neuraminic acid) and are consequently negatively charged. Although ubiquitous, gangliosides are abundant on the cell surface of the peripheral and central nervous system (CNS) (Lloyd & Furukawa, Glycoconjugate J. (1998) 15:627-636). The majority of GSLs are glucose derivatives of ceramide. However, galactose based GSLs are also present and are particularly abundant in the CNS. Such galactose-based GSLs include the sulfatides.

There are several classes of compounds which can affect the metabolism of glycolipids, including compounds of formulae (I), (II), (III), (IV), (V), (IX) and (XII) defined above. Some of these compounds (notably, NB-DNJ) have found use in the treatment of congenital disorders of glycolipid storage (such as type I Gaucher disease, reviewed in Aerts J M et al. J. Inherit Metab. Dis. (2006) 29(2-3): 449-453), or as potential anti-microbial agents (for example to modulate the toxicity of cholera toxin to ganglioside-type glycolipids, reviewed in Svensson M et al. Mol. Microbiol. (2003) 47: 453-461).

Defects in GSL catabolism result in a build up of GSLs. Such diseases are termed GSL storage disorders. Small-molecule inhibitors such as the alkyl-iminosugars have been developed to inhibit the biosynthesis of glucosylceramide, the first step in the biosynthesis of GSLs. Such compounds are thus inhibitors of glycolipid biosynthesis which may be employed in the present invention. Glucosylceramide is synthesised by the action of glucosylceramide synthase (also known as UDP-glucose: N-acylsphingosine glucosyltransferase), which catalyses the transfer of glucose to ceramide. The inhibition of glucosylceramide synthase can be achieved in vivo by small-molecule inhibitors (Reviewed in Asano N. Glycobiology (2003) 13:93-104). Inhibition can be achieved by small-molecule mimics of the substrate, transition state or product of glucosylceramide synthase. Broadly, three classes of inhibitors can be deduced: (1) mimics of the carbohydrate moiety (“sugar mimics”), (2) mimics of the ceramide or sphingosine moiety (“lipid mimics”) and (3) mimics of the nucleotide moiety of the sugar-nucleotide substrate of the glycosyltransferase (“nucleotide mimics”). Many inhibitors exhibit properties of more than one class. For example, inhibitors can exhibit properties of both (1) and (2) (e.g. Alkylated-DNJ, and AMP-DNJ, discussed below).

The sugar mimics (1) have received considerable attention and include iminosugars such as nojirimycin, N-butyldeoxynojirimycin (NB-DNJ) and N-butyldeoxygalactonojirimycin (NB-DGJ) (see U.S. Pat. No. 5,472,969; U.S. Pat. No. 5,656,641; U.S. Pat. No. 6,465,488; U.S. Pat. No. 6,610,703; U.S. Pat. No. 6,291,657; U.S. Pat. No. 5,580,884; Platt F, S. Biol. Chem. (1994) 269:8362-8365; Platt F M et al. Phil. Trans. R. Soc. Lond. B (2003) 358:947-954; and Butters T D et al. Glycobiology (2005) 15:43-52). The modification of the iminosugar core with an alkyl chain such as a butyl group (as in NB-DNJ) or a nonyl group (as in N,N-DNJ) are important for the clinical applications. Further sugar derivatives include N-(5-adamantane-1-yl-methoxypentyl)-DNJ (AMP-DNJ) (Overkleeft et al. J. Biol. Chem. (1998) 41:26522-26527), α-homogalactonojirimycin (HGJ) (Martin et al. 1995 Tetrahedron Letters 36:10101-10116), α-homoallonojirimycin (HAJ) (Asano et al 1997 J. Nat. Prod. 60:98-101, Martin et al 1999 Bioorg. Med. Chem. Lett 9:3171-3174) and β-1-C-butyl-DGJ (CB-DGJ) (Ikeda et al 2000 Carbohydrate Res. 323:73-80). NB-DNJ results in measurable decrease in GSL levels within a day of treatment with the effect on GSL levels stabilizing after 10 days of treatment in mice (Platt F M J. Biol. Chem. (1997) Aug. 1; 272(31):19365-72.). Critically, both NB-DNJ and NB-DGJ penetrate the CNS without significant effects on behaviour or CNS development, and treatment of adult mice with NB-DNJ or NB-DGJ has been shown not to cause neurological side effects (U. Andersson et al., Neurobiology of Disease, 16 (2004) 506-515). NB-DGJ resulted in a marked reduction in total ganglioside and GM1 content in cerebrum-brainstem (Kasperzyk et al. J. Lipid Res. (2005) 46:744-751). It is believed that, as distinct from their known efficacy in reducing lysosomal storage of glycolipids, these compounds could be used to disrupt or remove auto-immune epitopes from the cell surface. Glycolipids are a target for autoantibodies in many autoimmune conditions, as discussed herein below.

More recently, lipid mimics (2) have been developed to inhibit glycolipid biosynthesis (Abe A. et al. J. Biochem Tokyo (1992) 111:191-196. Reviewed in Asano N. Glycobiology (2003) 13:93-104). Ceramide-based inhibitors include D,L-threo-1-phenyl-2-decanoylamino-3-morpholino-1-propanol (DMP) and D,L-threo-1-phenyl-2-hexadecanoylamino-3-morpholino-1-propanol (PPMP). Numerous derivatives have subsequently been developed such as: D-threo-1-phenyl-2-palmitoilamino-3-pyrrolidino-1-propanol (P4), 4′-hydroxy-P4 (pOH-P4) and 3′,4′-ethylenedioxy-P4 (EtDO-P4; Genz-78132, Genzyme). L-DMDP (Yu C Y et al. Chem Commun (Camb). 2004 Sep. 7; (17):1936-7). Small-molecule inhibitors of galactosyltransferases have also been developed and are described in Chung S J, Bioorg Med Chem. Lett. 1998 Dec. 1; 8(23):3359-64.

Typically, the inhibitor of glycolipid biosynthesis is an inhibitor of glycolipid biosynthesis other than D,L-threo-1-phenyl-2-decanoylamino-3-morpholino-1-propanol (PDMP).

Glycolipid biosynthesis can also be disrupted by the use of small molecule inhibitors of the glycosidases, glycosyltransferases and other enzymes such as transferases and synthases, that act upstream or downstream of glucosylceramide synthase or galactosylceramide synthase. Such inhibitors are capable of inhibiting the synthesis of a glycolipid and are therefore inhibitors of glycolipid biosynthesis which may be employed in the present invention.

Inhibitors of the glycosidases and glycosyltransferases that act downstream of the glucosylceramide synthase or galactosylceramide synthase may be employed in the present invention. For example the biosynthesis of sialic acid containing glycolipids, the gangliosides can be downregulated by the use of inhibitors of the sialyltransferases. Example compounds include, sialic acid (N-acetylneuraminic acid), lithocholic acid analogues which potently inhibit α2-3-sialyltransferase (Chang K H et al. Chem Commun (Camb). 2006 Feb. 14; (6):629-31), cytidin-5′-yl sialylethylphosphonate which inhibits rat recombinant α-2,3- and α-2,6-ST (IC(50)=0.047, 0.34 mM (Izumi M J Org Chem. 2005 Oct. 28; 70(22):8817-24), and Soyasaponin I, a potent and specific sialyltransferase inhibitor (Wu C Y Biochem Biophys Res Commun. 2001 Jun. 8; 284(2):466-9). Furthermore, some carbohydrates are modified by the addition or removal of chemical groups from the glycan backbone. For example sulfatides are formed by the action of sulfotransferase on carbohydrate moiety of glycolipids. These enzymes are themselves targets for reduction of autoreactive antigens.

Accordingly, in one embodiment of the invention the inhibitor of glycolipid biosynthesis is an inhibitor of a glycosyltransferase. Typically, the inhibitor mimics the substrate, transition state or product of the glycosyltransferase. In particular, the inhibitor may be a compound that mimics the carbohydrate moiety of the substrate, transition state or product of the glycosyltransferase. Alternatively, the inhibitor is a compound that mimics the lipid moiety of the substrate, transition state or product of the glycosyltransferase. Alternatively, the inhibitor is a compound that mimics the nucleotide moiety of the sugar-nucleotide substrate or transition state of the glycosyltransferase. Typically, the glycosyltransferase is a glucosyltransferase. The glucosyltransferase is, for instance, glucosylceramide synthase. Alternatively, the glycosyltransferase may be a galactosyltransferase. The galactosyltransferase may be, for instance, β-4 galactosyltransferase. Alternatively, the glycosyltransferase may be a ceramide galactosyltransferase. The ceramide galactosyltransferase may be, for instance, UDP-galactose:ceramide galactosyltransferase. (also known as galactosylceramide synthase). Alternatively, the glycosyltransferase is a sialyltransferase.

In principle, all glycosyltransferases can be inhibited by substrate mimics (Chung S J, Bioorg Med Chem. Lett. 1998 Dec. 1; 8(23):3359-64). Such substrate mimics can be employed for use in the present invention as inhibitors of glycolipid biosynthesis.

Further examples of inhibitors of glycolipid biosynthesis include inhibitors of sulfotransferase, fucosyltransferase, or N-acetylhexosaminetransferase. Thus in one embodiment of the present invention, the inhibitor of glycolipid biosynthesis is an inhibitor of a sulfotransferase. In another embodiment of the present invention, the inhibitor of glycolipid biosynthesis is an inhibitor of a fucosyltransferase. In another embodiment of the present invention, the inhibitor of glycolipid biosynthesis is an inhibitor of an N-acetylhexosaminetransferase. Sulfotransferase inhibitors are described in Armstrong, J. I. et al. Angew. Chem. Int. Ed. 2000, 39, No. 7, 1303-1306 and references therein. Examples of sulfotransferase inhibitors are given in Table 3 below. In one embodiment, the inhibitor of glycolipid biosynthesis is an inhibitor of a glycosyltransferase or a sulfotransferase. Fucosyltransferase inhibitors are described in Qiao, L. et al., S. Am. Chem. Soc. 1996, 118, 7653-7662. An example of a fucosyltransferase inhibitor is propyl 2-acetamido-2-deoxy-4-O-(β-D-galactopyranosyl)-3-O-(2-(N-(β-L-homofaconojirimycinyl))ethyl)-α-D-glucopyranoside, which is an azatrisaccharide compound. Qiao, L. et al. found that compound, in the presence of guanosine diphosphate (GDP), to be an effective inhibitor of human α-1,3-fucosyltransferase V. In addition, Wong, C-H, Pure & Appl. Chem., Vol. 67, No. 10, pp 1609-1616 describes the synergistic inhibition of α-1,3-fucosyltransferase using an azasugar and GDP. Azasugar compounds (such as those of formula I herein) can be employed for use in the present invention as inhibitors of glycolipid biosynthesis. Such azasugar compounds can be used as inhibitors of glycolipid biosynthesis either alone or in combination with a nucleotide mimic compound, for instance in combination with GDP or a compound of formula III herein. N-acetylhexosaminetransferase inhibitors are described in Schäfer et al., J. Org. Chem. 2000, 65, 24-29. Compounds 58a to 58c and 59a to 59c in Table 2 below are examples of N-acetylhexosaminetransferase inhibitors.

Glycolipid biosynthesis can be disrupted by the use of inhibitors of enzymes, such as transferases and synthases, that act upstream of glucosylceramide synthase or galactosylceramide synthase. Such inhibitors are termed “inhibitors of ceramide biosynthesis”. Inhibitors of ceramide biosynthesis are capable of inhibiting the synthesis of a glycolipid and are therefore inhibitors of glycolipid biosynthesis which may be employed in the present invention.

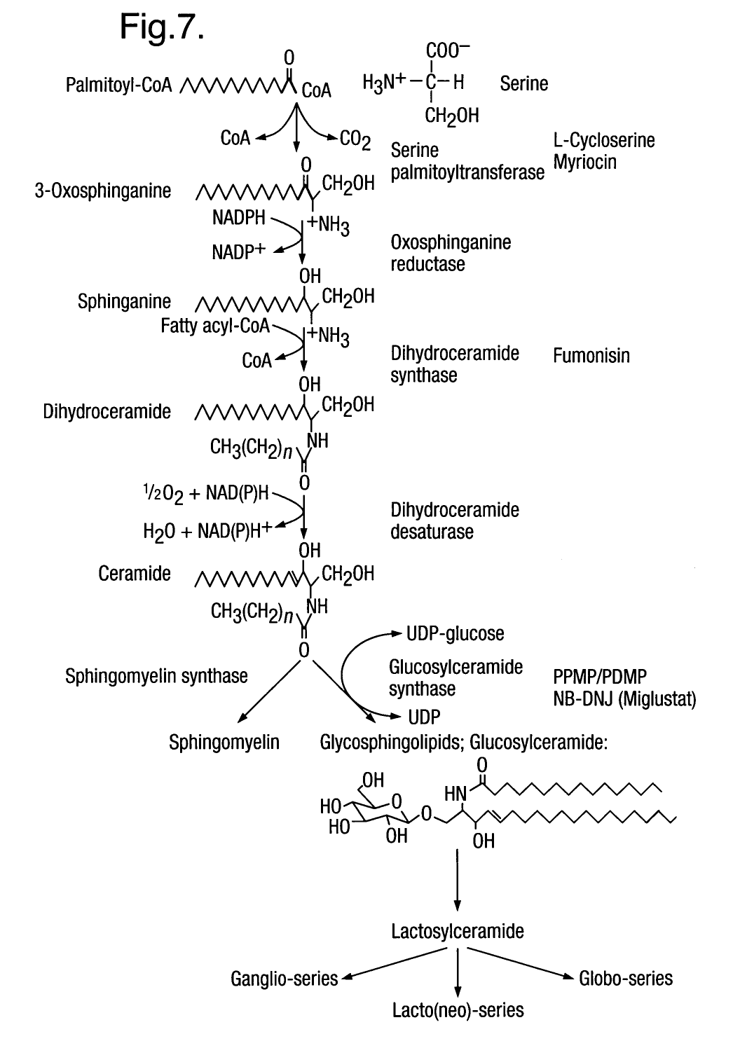

Enzymes which act upstream of glucosylceramide synthase include serine palmitoyltransferase and dihydroceramide synthase. Inhibitors of serine palmitoyltransferase include L-Cycloserine and Myriocin. Inhibitors of dihydroceramide synthase include Fumonisin. These particular enzymes and inhibitors of ceramide biosynthesis are indicated in the schematic diagram of glycosphingolipid biosynthesis in FIG. 7.

In one embodiment, the inhibitor of glycolipid biosynthesis is an inhibitor of ceramide biosynthesis. Typically, the inhibitor of glycolipid biosynthesis is an inhibitor of serine palmitoyltransferase or an inhibitor of dihydroceramide synthase. More typically, in this embodiment, the inhibitor of glycolipid biosynthesis is L-Cycloserine, Myriocin or Fumonisin.

Typically, the inhibitor of glycolipid biosynthesis is an inhibitor of glycolipid biosynthesis other than an inhibitor of a sialyltransferase.

The skilled person can readily identify inhibitors of glycolipid biosynthesis without undue experimentation, using known procedures. For instance, inhibitors of glycolipid biosynthesis can be identified by incubating and or growing cells in culture in the presence of the putative inhibitor together with an assay for the effect of glycolipid biosynthesis. Such assays include the analysis of fluorescently-labelled glycolipid carbohydrate headgroups by HPLC, thin-layer chromatography (TLC) of glycolipids and analysis of glycolipids using mass spectrometry (Neville D C, Anal. Biochem. 2004 Aug. 15; 331(2):275-82; Mellor H R Biochem. J. 2004 Aug. 1; 381 (Pt 3):861-6; Hayashi Y. et al., Anal. Biochem. 2005 Oct. 15; 345(2):181-6; Sandhoff, R. et al., J. Biol. Chem., vol. 277, no. 23, 20386-20398, 2002; Sandhoff, R. et al., J. Biol. Chem., vol. 280, no. 29, 27310-27318, 2005; Platt, F. M. et al., J. Biol. Chem., vol. 269, issue 11, 8362-8365, 1994; Platt, F. M. et al., J. Biol. Chem., vol. 269, issue 43, 27108-27114, 1994).

Neville D C et al. (Anal. Biochem. 2004 Aug. 15; 331(2):275-82) have developed an optimised assay method in which fluorescently labelled glycosphingolipid-derived oligosaccharides are analysed. Thus, inhibitors of glycolipid biosynthesis for use in accordance with the present invention can be identified by incubating or growing cells in culture, in the presence of the putative inhibitor, and applying the assay described in Neville et al. The assay described in Neville et al. enables GSL levels to be measured by HPLC analysis of GSL-derived oligosaccharides following ceramide glycanase digestion of the GSLs and anthranilic acid labelling of the released oligosaccharides. In the assay, glyocosphingolipids (GSLs) are extracted from the sample and purified by column chromatography. The extracted GSLs are then digested with ceramide glycanase. The extracted GSLs are first dried and redissolved, with mixing in 10 μl incubation buffer (50 μM sodium acetate, pH 5.0, containing 1 mg/ml sodium cholate or sodium taurodeoxycholate). To this is added, with gentle mixing, 0.05 U ceramide glycanase in a further 10 μl incubation buffer (giving a final concentration of 2.5 U/ml). One unit (U) is defined as the amount of enzyme that will hydrolyze 1.0 nmol of ganglioside, GM1, per minute at 37° C. Incubations are performed at 37° C. for 18 hours. The ceramide-glycanase-released oligosaccharides are then labelled with anthranilic acid and purified essentially as described in Anumula and Dhume, Glycobiology 8 (1998) 685-694 with the modifications described in Neville D C et al. Anal. Biochem. 2004 Aug. 15; 331(2):275-82. The purified 2-AA-labelled oligosaccharides are then separated by normal phase HPLC, as described in Neville D C et al., and glucose unit values are determined following comparison with a 2-AA-labelled glucose oligomer ladder (derived from a partial hydrolysate of dextran) external standard. Inhibitors of glycolipid biosynthesis are identified by measuring the decrease in GSL levels observed in the presence of the inhibitor. A similar assay method is described in Mellor H R Biochem. J. 2004 Aug. 1; 381(Pt 3):861-6. That document describes the synthesis of a series of DNJ analogues to study their inhibitory activity in cultured HL60 cells. When the cells are treated for 16 hours at non-cytotoxic concentrations of DNJ analogue, a 40-50% decrease in GSL levels can be observed by HPLC analysis of GSL-derived oligosaccharides following ceramide glycanase digestion of GSL and 2-aminobenzamide labelling of the released oligosaccharides.

Hayashi Y. et al., Anal. Biochem. 2005 Oct. 15; 345(2):181-6 reports an HPLC-based method that uses fluorescent acceptors and nonradioisotope UDP-sugar donors to provide a fast, sensitive and reproducible assay to determine glucosylceramide synthase (GlcT) and lactosylceramide synthase (GalT) activities. Thus, inhibitors of glycolipid biosynthesis for use in accordance with the present invention can be identified by incubating and or growing cells in culture in the presence of the putative inhibitor, and applying the assay method described in Hayashi et al. The HPLC-based assay procedures described in Hayashi et al. involve mixing a fluorescent acceptor substrate, either 50 μmol of C6-NBD-Cer or C6-NBD-GlcCer, and 6.5 nmol of lecithin in 100 μmol of ethanol, and then evaporating the solvent. Next 10 μl of water is added and the mixture is sonicated to form liposomes. For the GlcT assay, 50 μl of reaction mixture contains 500 μM UDP-Glc, 1 mM EDTA, 10 μl C6-NBD-Cer liposome and 20 μl of an appropriate amount of enzyme in lysis buffer 1. For the GalT assay, 50 μl of mixture contains 100 μM UDP-Gal, 5 mM MgCl₂, 5 mM MnCl₂, 10 μl C6-NBD-GlcCer liposome, and 20 μl of an appropriate amount of enzyme in lysis buffer 2. The assays are carried out at 37° C. for 1 hour. The reaction is stopped by adding 200 μl of chloroform/methanol (2:1, v/v). After a few seconds of vortexing, 5 μl of 500 μM KCl is added and then centrifuged. After the organic phase has dried up, lipids are dissolved in 200 μl of isopropyl alcohol/n-hexane/H₂O (55:44:1) and then transferred to a glass vial in an autosampler. A 100 μl aliquot sample is then loaded onto a normal-phase column and eluted with isopropyl alcohol/n-hexane/H₂O (55:44:1) for the GlcT assay or isopropyl alcohol/n-hexane/H₂O/phosphoric acid (110:84:5.9:0.1) for the GalT assay at a flow rate of 2.0 ml/min. Fluorescence can be determined using a fluorescent detector set to excitation and emission wavelengths of 470 and 530 nm, respectively. Fluorescent peaks are identified by comparing their retention times with those of standards.

Further assays include fluorescent-activated cells sorting (FACS) with glycolipid-binding proteins such as anti-glycolipid antibodies or lectins (see for example Rouquette-Jazdanian et al., The Journal of Immunology, 2005, 175: 5637-5648 and Chefalo, P et al., Biochemistry 2006, Mar. 21; 45(11): 3733-9).

Sandhoff et al. (J. Biol. Chem., vol. 277, no. 23, 20386-20398, 2002 and J. Biol. Chem., vol. 280, no. 29, 27310-27318, 2005) describe assay methods in which glycolipids are analysed by mass spectrometry or by TLC. Inhibitors of glycolipid biosynthesis for use in accordance with the present invention can be identified by incubating and or growing cells in culture in the presence of the putative inhibitor, and applying the TLC assay method or mass spectrometry assay method described by Sandhoff et al. Further details of these methods are given below.

In the methods described by Sandhoff et al. (J. Biol. Chem., vol. 277, no. 23, 20386-20398, 2002 and J. Biol. Chem., vol. 280, no. 29, 27310-27318, 2005) the glycosphingolipid profiles in mice were measured by nano-electrospray ionization tandem mass spectrometry (nano-ESI-MS/MS). Glycosphingolipids were first extracted from murine tissue for mass spectrometric analysis. The samples prepared included both the extracted GSLs and synthesised GSL MS standards. Nano-ESI-MS/MS analyses were performed with a triple quadropole instrument equipped with a nano-electrospray source operating at an estimated flow rate of 20-50 nl/min. Usually 10 μL of sample, dissolved in methanol or methanolic ammonium acetate (5 mM), was filled into a gold-sputtered capillary, which was positioned at a distance of 1-3 mm in front of the cone. The source temperature was set to 30° C. and the spray was started by applying 800-1200 V to the capillary. For each spectrum 20-50 scans of 15-30 s duration were averaged. The resulting Nano-ESI-MS/MS data could then be evaluated for quantification of the GSLs as follows: Quantitative spectra were measured with an average mass resolution of 1200 (ion mass/full width half maximum). Peak height values of the first mono-isotopic peak of each compound were taken for evaluation. A linear trend was calculated from the peak intensities of the corresponding internal standard lipids. The obtained calibration curve represented the intensity of the internal standard amount at a given m/z value. The quantities of the individual species of a GSL were calculated using a corrected intensity ratio (sample GSL/internal standard trend), knowing the amount of the internal standard added. The amount of the GSL was then calculated from the sum of the individual molecular species.

Sandhoff et al. (J. Biol. Chem., vol. 277, no. 23, 20386-20398, 2002 and J. Biol. Chem., vol. 280, no. 29, 27310-27318, 2005) also describe a procedure for analysing GSLs using TLC. Glycosphingolipids were extracted from murine tissue for analysis by TLC. Neutral and acidic GSL fractions were each taken up in 100 μL chloroform/methanol/water (10:10:1). Aliquots were then spotted on TLC plates with a Linomat IV from CAMAG (Muttenz, C H). A pre-run was performed with chloroform/alcohol (1:1). The plates were then dried and the GSLs were separated with the running solvent chloroform, methanol, 0.2% aqueous CaCl₂ (60:35:8). GSL bands were detected with orcinol/sulphuric acid spray reagent at 110° C. for 10 to 20 mins and the GSLs were identified by comparison with GSL standards.

TLC assays for analysing glycolipid biosynthesis are also described in Platt, F. M. et al., J. Biol. Chem., vol. 269, issue 11, 8362-8365 and 1994; Platt, F. M. et al., J. Biol. Chem., vol. 269, issue 43, 27108-27114, 1994.

In another embodiment, the inhibitor of glycolipid biosynthesis is Ribonucleic acid (RNA). RNA can be used to reduce (“knock down”) expression of a target enzyme which is involved in glycolipid biosynthesis, such as a transferase enzyme, in order to achieve the same result as a small molecule inhibitor of that enzyme. The transferase enzyme may be a glycosyltransferase, for instance. Typically, the transferase enzyme is a glucosyltransferase, sialyltransferase, galactosyltransferasae, ceramide galactosyltransferase, sulfotransferase, faicosyltransferase, or an N-acetylhexosaminetransferase. In one embodiment, the transferase enzyme is a galactosyltransferase, for instance α-1,3-galactosyltransferase. Typically the RNA is antisense RNA or siRNA (small interfering RNA).

The skilled person can readily identify RNA inhibitors of glycolipid biosynthesis without undue experimentation, using known procedures. By considering the coding sequence of a particular target enzyme which is involved in glycolipid biosynthesis, the skilled person is able to design RNA, for instance antisense RNA or siRNA, that is able to reduce (“knock down”) expression of that enzyme (see, for example, Huesken, D. et al. (2005) Design of a genome-wide siRNA library using an artificial neural network. Nat. Biotechnol. 23, 995).

Zhu, M. et al., Transplantation 2005; 79: 289-296 describes the use of siRNA to reduce expression of the galactosyltransferase enzyme α-1,3-galactosyltransferase and, consequently, reduce synthesis of the α-Gal epitope (Galα1-3Galβ1-4GlcNAc-R). In Zhu et al., α-1,3-galactosyltransferase-specific siRNA was transfected into the porcine aortic endothelial cell line, PED. α-Gal expression was assessed by Western blotting, flow cytometric analyses (FACS) and immunofluorescence. RNA interference was successfully achieved in PED cells as shown by the specific knock-down of α1,3 galactosyltransferase mRNA levels. Flow cytometric analysis using the Griffonia simplicifolia isolectin B4 lectin confirmed the suppression of α-1,3-galactosyltransferase activity, as evidenced by decreased α-Gal.

The siRNA duplexes used by Zhu et al. were synthesised by in vitro transcription with T7 RNA polymerase and obtained readily annealed (Genesil, Wuhan, China). The duplexes were designed by considering the various isoforms of α-1,3-galactosyltransferase, termed α1,3GT isoforms 1, 2, 3, 4 and 5 respectively. These isoforms are a result of the alternative splicing of exons 5, 6 and 7 of α-1,3-galactosyltransferase. Porcine endothelial cells express isoforms 1, 2 and 4 only. The catalytic domain of α-1,3-galactosyltransferase is encoded by exons 7, 8 and 9. Thus, in order to avoid missing certain splice variants, and to efficaciously knockdown the expression of the α-1,3-galactosyltransferase mRNA translating the three PED isoforms simultaneously, two siRNA duplexes were sythesised that were specific for the α-1,3-galactosyltransferase mRNA sequence located in exons 7 and 9 as the target of siRNA. The siRNA duplex specific for the α-1,3-galactosyltransferase mRNA sequence located in exon 7 was termed “siRNA-1”, and the siRNA duplex specific for the α-1,3-galactosyltransferase mRNA sequence located in exon 9 was termed “siRNA-2”. Zhu et al. found siRNA-1 to be effective in reducing α-1,3-galactosyltransferase mRNA expression. The siRNA-1 sequence is from position +199 to +217 relative to the start codon of the porcine α-1,3-galactosyltransferase coding sequence (Genbank Accession No. AF221508). The sequence of the siRNA-1 duplex is as follows:

| | |
|---|---|
| sense: | 5′-GAAGAAGACGCUAUAGGCAdTdT-3′ |
| antisense: | 5′-UGCCUAUAGCGUCUUCUUCCdTdT-3′ |

According to Zhu, M. et al., FACS analysis and immunofluorescent assay indicated that the transfection with siRNA-1 led to a dramatic decrease in binding of fluorescein isothiocyanate-conjugated Griffonia simplicifolia isolectin B4 (FITC-GS-IB4) to the α-Gal epitope as compared with parental PED, indicating that decreased α-Gal expression had occurred. Western Blot analysis further confirmed the α-1,3-galactosyltransferase RNA interference effect on the synthesis of Glycoproteins which have the α-Gal residue.

Typically, the inhibitor of glycolipid biosynthesis is an inhibitor of glycolipid biosynthesis other than ribonucleic acid (RNA).

The following definitions apply to the compounds of formula (I) and formula (II):

A C₁₋₂₀ alkyl group is an unsubstituted or substituted, straight or branched chain saturated hydrocarbon radical. Typically it is C₁₋₁₀ alkyl, for example methyl, ethyl, propyl, butyl, pentyl, hexyl, heptyl, octyl, nonyl or decyl, or C₁₋₆ alkyl, for example methyl, ethyl, propyl, butyl, pentyl or hexyl, or C₁₋₄ alkyl, for example methyl, ethyl, i-propyl, n-propyl, t-butyl, s-butyl or n-butyl. When an alkyl group is substituted it typically bears one or more substituents selected from substituted or unsubstituted C₁₋₂₀ alkyl, substituted or unsubstituted aryl (as defined herein), cyano, amino, C₁₋₁₀ alkylamino, di(C₁₋₁₀)alkylamino, arylamino, diarylamino, arylalkylamino, amido, acylamido, hydroxy, oxo, halo, carboxy, ester, acyl, acyloxy, C₁₋₂₀ alkoxy, aryloxy, haloalkyl, sulfonic acid, sulfhydryl (i.e. thiol, —SH), C₁₋₁₀ alkylthio, arylthio, sulfonyl, phosphoric acid, phosphate ester, phosphonic acid and phosphonate ester. Examples of substituted alkyl groups include haloalkyl, hydroxyalkyl, aminoalkyl, alkoxyalkyl and alkaryl groups. The term alkaryl, as used herein, pertains to a C₁₋₂₀ alkyl group in which at least one hydrogen atom has been replaced with an aryl group. Examples of such groups include, but are not limited to, benzyl(phenylmethyl, PhCH₂—), benzhydryl(Ph₂CH—), trityl(triphenylmethyl, Ph₃C—), phenethyl(phenylethyl, Ph-CH₂CH₂—), styryl(Ph-CH═CH—), cinnamyl(Ph-CH═CH—CH₂—).

Typically a substituted C₁₋₂₀ alkyl group carries 1, 2 or 3 substituents, for instance 1 or 2.

A C₃₋₂₅ cycloalkyl group is an unsubstituted or substituted alkyl group which is also a cyclyl group; that is, a monovalent moiety obtained by removing a hydrogen atom from an alicyclic ring atom of a carbocyclic ring of a carbocyclic compound, which moiety has from 3 to 25 carbon atoms (unless otherwise specified), including from 3 to 25 ring atoms. Thus, the term “cycloalkyl” includes the sub-classes cycloalkyenyl and cycloalkynyl. Examples of groups of C₃₋₂₅ cycloalkyl groups include C₃₋₂₀ cycloalkyl, C₃₋₁₅ cycloalkyl, C₃₋₁₀ cycloalkyl, C₃₋₇ cycloalkyl. When a C₃₋₂₅ cycloalkyl group is substituted it typically bears one or more substituents selected from C₁₋₆ alkyl which is unsubstituted, aryl (as defined herein), cyano, amino, C₁₋₁₀ alkylamino, di(C₁₋₁₀)alkylamino, arylamino, diarylamino, arylalkylamino, amido, acylamido, hydroxy, oxo, halo, carboxy, ester, acyl, acyloxy, C₁₋₂₀ alkoxy, aryloxy, haloalkyl, sulfonic acid, sulfhydryl (i.e. thiol, —SH), C₁₋₁₀ alkylthio, arylthio, phosphoric acid, phosphate ester, phosphonic acid and phosphonate ester and sulfonyl. Typically a substituted C₃₋₂₅ cycloalkyl group carries 1, 2 or 3 substituents, for instance 1 or 2.

Examples of C₃₋₂₅ cycloalkyl groups include, but are not limited to, those derived from saturated monocyclic hydrocarbon compounds, which C₃₋₂₅ cycloalkyl groups are unsubstituted or substituted as defined above:

cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇), methylcyclohexane (C₇), dimethylcyclohexane (C₈), menthane (C₁₀);

unsaturated monocyclic hydrocarbon compounds:

cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇), methylcyclohexene (C₇), dimethylcyclohexene (C8);

saturated polycyclic hydrocarbon compounds:

thujane (C₁₀), carane (C₁₀), pinane (C₁₀), bornane (C₁₀), norcarane (C₇), norpinane (C₇), norbornane (C₇), adamantane (C₁₀), decalin (decahydronaphthalene) (C₁₀);

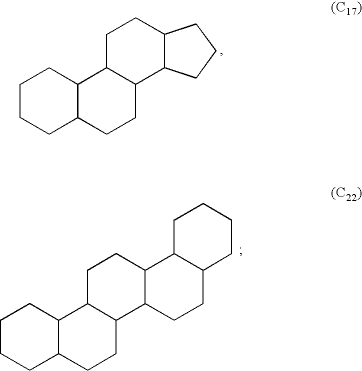

unsaturated polycyclic hydrocarbon compounds: camphene (C₁₀), limonene (C₁₀), pinene (C₁₀),

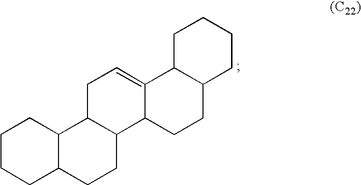

polycyclic hydrocarbon compounds having an aromatic ring:

indene (C₉), indane (e.g., 2,3-dihydro-1H-indene) (C₉), tetraline (1,2,3,4-tetrahydronaphthalene) (C₁₀C), acenaphthene (C₁₂), fluorene (C₁₃), phenalene (C₁₃), acephenanthrene (C₁₅), aceanthrene (C₁₆), cholanthrene (C₂₀).

A C₃₋₂₀ heterocyclyl group is an unsubstituted or substituted monovalent moiety obtained by removing a hydrogen atom from a ring atom of a heterocyclic compound, which moiety has from 3 to 20 ring atoms (unless otherwise specified), of which from 1 to 10 are ring heteroatoms. Preferably, each ring has from 3 to 7 ring atoms, of which from 1 to 4 are ring heteroatoms. When a C₃₋₂₀ heterocyclyl group is substituted it typically bears one or more substituents selected from C₁₋₆ alkyl which is unsubstituted, aryl (as defined herein), cyano, amino, C₁₋₁₀ alkylamino, di(C₁₋₁₀)alkylamino, arylamino, diarylamino, arylalkylamino, amido, acylamido, hydroxy, oxo, halo, carboxy, ester, acyl, acyloxy, C₁₋₂₀ alkoxy, aryloxy, haloalkyl, sulfonic acid, sulfhydryl (i.e. thiol, —SH), C₁₋₁₀ alkylthio, arylthio, phosphoric acid, phosphate ester, phosphonic acid and phosphonate ester and sulfonyl. Typically a substituted C₃₋₂₀ heterocyclyl group carries 1, 2 or 3 substituents, for instance 1 or 2.

Examples of groups of heterocyclyl groups include C₃₋₂₀heterocyclyl, C₅₋₂₀heterocyclyl, C₃₋₁₅heterocyclyl, C₅₋₁₅heterocyclyl, C₃₋₁₂heterocyclyl, C₅₋₁₂heterocyclyl, C₃₋₁₀heterocyclyl, C₅₋₁₀heterocyclyl, C₃₋₇heterocyclyl, C₅₋₇heterocyclyl, and C₅₋₆heterocyclyl.

Examples of (non-aromatic) monocyclic C₃₋₂₀ heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);

S₁: thiirane (C₃), thietane (C₄), thiolane (tetrahydrothiophene) (C₅), thiane (tetrahydrothiopyran) (C₆), thiepane (C₇);

O₂: dioxolane (C₅), dioxane (C₆), and dioxepane (C₇);

O₃: trioxane (C₆);

N₂: imidazolidine (C₅), pyrazolidine (diazolidine) (C₅), imidazoline (C₅), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆);

N₂O₁: oxadiazine (C₆);

O₁S₁: oxathiole (C₅) and oxathiane (thioxane) (C₆); and,

N₁O₁S₁: oxathiazine (C₆).

Examples of substituted (non-aromatic) monocyclic heterocyclyl groups include those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose. C₃₋₂₀ heterocyclyl includes groups derived from heterocyclic compounds of the following structure:

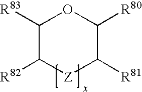

wherein x is 0 or 1; z is CHR⁸⁴; and R⁸⁰, R⁸¹, R⁸², R⁸³ and R⁸⁴, which are the same or different, are independently selected from H, C₁₋₆ alkyl, OH, acyloxy, SH, C₁₋₆ alkoxy, aryloxy, amino, C₁₋₁₀ alkylamino, di(C₁₋₁₀)alkylamino, amido, acylamido and a group derived from a second group of the following structure:

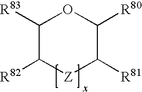

in which second group x is 0 or 1; z is CHR⁸⁴; and R⁸⁰, R⁸¹, R², R⁸³ and R⁸⁴, which are the same or different, are independently selected from H, C₁₋₆ alkyl, OH, acyloxy, SH, C₁₋₆ alkoxy, aryloxy, amino, C₁₋₁₀ alkylamino, di(C₁₋₁₀)alkylamino, amido and acylamido. The term “group derived from” in this case means that the group is a monovalent moiety obtained by removing the R⁸⁰, R⁸¹, R⁸², R⁸³ or R⁸⁴ atom from a carbon atom of the above compounds. Thus, C₃₋₂₀ heterocyclyl includes groups of the following structure:

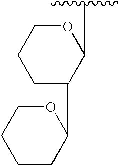

wherein each of the ring carbon atoms is independently unsubstituted or substituted with C₁₋₆ alkyl, OH, acyloxy, SH, C₁₋₆ alkoxy, aryloxy, amino, C₁₋₁₀ alkylamino, di(C₁₋₁₀)alkylamino, amido and acylamido.

C₃₋₂₀ heterocyclyl also includes groups in which two heterocyclic rings are linked by an oxygen atom. Thus, C₃₋₂₀ heterocyclyl includes disaccharide groups, in which two monosaccharide heterocyclic rings are linked with an oxygen atom. Accordingly, C₃₋₂₀ heterocyclyl includes groups of the following formula (m):

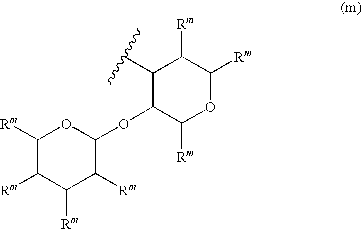

wherein each R^{m}, which is the same or different, is independently selected from C₁₋₆ alkyl, OH, acyloxy, SH, C₁₋₆ alkoxy, aryloxy, amino, C₁₋₁₀ alkylamino, di(C₁₋₁₀)alkylamino, amido and acylamido. Thus, the following disaccharide group is one example of a substituted C₃₋₂₀ heterocyclic group:

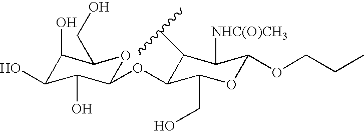

Examples of C₃₋₂₀ heterocyclyl groups which are also aryl groups are described below as heteroaryl groups.

An aryl group is a substituted or unsubstituted, monocyclic or bicyclic aromatic group which typically contains from 6 to 14 carbon atoms, preferably from 6 to 10 carbon atoms in the ring portion. Examples include phenyl, naphthyl, indenyl and indanyl groups. An aryl group is unsubstituted or substituted. When an aryl group as defined above is substituted it typically bears one or more substituents selected from C₁-C₆ alkyl which is unsubstituted (to form an aralkyl group), aryl which is unsubstituted, cyano, amino, C₁₋₁₄₀ alkylamino, di(C₁₋₁₀)alkylamino, arylamino, diarylamino, arylalkylamino, amido, acylamido, hydroxy, halo, carboxy, ester, acyl, acyloxy, C₁₋₂₀ alkoxy, aryloxy, haloalkyl, sulfhydryl (i.e. thiol, —SH), C₁₋₁₀ alkylthio, arylthio, sulfonic acid, phosphoric acid, phosphate ester, phosphonic acid and phosphonate ester and sulfonyl. Typically it carries 0, 1, 2 or 3 substituents. A substituted aryl group may be substituted in two positions with a single C₁₋₆ alkylene group, or with a bidentate group represented by the formula —X—C₁₋₆ alkylene, or —X—C₁₋₆ alkylene-X—, wherein X is selected from O, S and NR, and wherein R is H, aryl or C₁₋₆ alkyl. Thus a substituted aryl group may be an aryl group fused with a cycloalkyl group or with a heterocyclyl group. The term aralkyl as used herein, pertains to an aryl group in which at least one hydrogen atom (e.g., 1, 2, 3) has been substituted with a C₁₋₆ alkyl group. Examples of such groups include, but are not limited to, tolyl (from toluene), xylyl (from xylene), mesityl (from mesitylene), and cumenyl (or cumyl, from cumene), and duryl (from durene). The ring atoms of an aryl group may include one or more heteroatoms (as in a heteroaryl group). Such an aryl group (a heteroaryl group) is a substituted or unsubstituted mono- or bicyclic heteroaromatic group which typically contains from 6 to 10 atoms in the ring portion including one or more heteroatoms. It is generally a 5- or 6-membered ring, containing at least one heteroatom selected from O, S, N, P, Se and Si. It may contain, for example, 1, 2 or 3 heteroatoms. Examples of heteroaryl groups include pyridyl, pyrazinyl, pyrimidinyl, pyridazinyl, furanyl, thienyl, pyrazolidinyl, pyrrolyl, oxazolyl, oxadiazolyl, isoxazolyl, thiadiazolyl, thiazolyl, isothiazolyl, imidazolyl, pyrazolyl, quinolyl and isoquinolyl. A heteroaryl group may be unsubstituted or substituted, for instance, as specified above for aryl. Typically it carries 0, 1, 2 or 3 substituents.

A C₁₋₂₀ alkylene group is an unsubstituted or substituted bidentate moiety obtained by removing two hydrogen atoms, either both from the same carbon atom, or one from each of two different carbon atoms, of a hydrocarbon compound having from 1 to 20 carbon atoms (unless otherwise specified), which may be aliphatic or alicyclic, and which may be saturated, partially unsaturated, or fully unsaturated. Thus, the term “alkylene” includes the sub-classes alkenylene, alkynylene, cycloalkylene, etc., discussed below. Typically it is C₁₋₁₀ alkylene, for instance C₁₋₆ alkylene. Typically it is C₁₋₄ alkylene, for example methylene, ethylene, i-propylene, n-propylene, t-butylene, s-butylene or n-butylene. It may also be pentylene, hexylene, heptylene, octylene and the various branched chain isomers thereof. An alkylene group may be unsubstituted or substituted, for instance, as specified above for alkyl. Typically a substituted alkylene group carries 1, 2 or 3 substituents, for instance 1 or 2.

In this context, the prefixes (e.g., C₁₋₄, C₁₋₇, C₁₋₂₀, C₂₋₇, C₃₋₇, etc.) denote the number of carbon atoms, or range of number of carbon atoms. For example, the term “C₁₋₄alkylene,” as used herein, pertains to an alkylene group having from 1 to 4 carbon atoms. Examples of groups of alkylene groups include C₁₋₄ alkylene (“lower alkylene”), C₁₋₇ alkylene, C₁₋₁₀ alkylene and C₁₋₂₀ alkylene.

Examples of linear saturated C₁₋₇ alkylene groups include, but are not limited to, —(CH₂)ₙ— where n is an integer from 1 to 7, for example, —CH₂— (methylene), —CH₂CH₂-(ethylene), —CH₂CH₂CH₂— (propylene), and —CH₂CH₂CH₂CH₂— (butylene).

Examples of branched saturated C₁₋₇ alkylene groups include, but are not limited to, —CH(CH₃)—, —CH(CH₃)CH₂—, —CH(CH₃)CH₂CH₂—, —CH(CH₃)CH₂CH₂CH₂—, —CH₂CH(CH₃)CH₂—, —CH₂CH(CH₃)CH₂CH₂—, —CH(CH₂CH₃)—, —CH(CH₂CH₃)CH₂—, and —CH₂CH(CH₂CH₃)CH₂—

Examples of linear partially unsaturated C₁₋₇ alkylene groups include, but is not limited to, —CH═CH— (vinylene), —CH═CH—CH₂—, —CH₂—CH═CH₂—, —CH═CH—CH₂—CH₂—, —CH═CH—CH₂—CH₂—CH₂—, —CH═CH—CH═CH—, —CH═CH—CH═CH—CH₂—, —CH═CH—CH═CH—CH₂—CH₂—, —CH═CH—CH₂—CH═CH—, and —CH═CH—CH₂—CH₂—CH═CH—.

Examples of branched partially unsaturated C₁₋₇ alkylene groups include, but is not limited to, —C(CH₃)═CH—, —C(CH₃)═CH—CH₂—, and —CH═CH—CH(CH₃)—.

Examples of alicyclic saturated C₁₋₇ alkylene groups include, but are not limited to, cyclopentylene (e.g., cyclopent-1,3-ylene), and cyclohexylene (e.g., cyclohex-1,4-ylene).

Examples of alicyclic partially unsaturated C₁₋₇ alkylene groups include, but are not limited to, cyclopentenylene (e.g., 4-cyclopenten-1,3-ylene), cyclohexenylene (e.g., 2-cyclohexen-1,4-ylene; 3-cyclohexen-1,2-ylene; 2,5-cyclohexadien-1,4-ylene).

C₁₋₂₀ alkylene and C₁₋₂₀ alkyl groups as defined herein are either uninterrupted or interrupted by one or more heteroatoms or heterogroups, such as S, O or N(R″) wherein R″ is H, C₁₋₆ alkyl or aryl (typically phenyl), or by one or more arylene (typically phenylene) groups. The phrase “optionally interrupted” as used herein thus refers to a C₁₋₂₀ alkyl group or an alkylene group, as defined above, which is uninterrupted or which is interrupted between adjacent carbon atoms by a heteroatom such as oxygen or sulfur, by a heterogroup such as N(R″) wherein R″ is H, aryl or C₁-C₆ alkyl, or by an arylene group. For instance, a C₁₋₂₀ alkyl group such as n-butyl may be interrupted by the heterogroup N(R″) as follows: —CH₂N(R″)CH₂CH₂CH₃, —CH₂CH₂N(R″)CH₂CH₃, or —CH₂CH₂CH₂N(R″)CH₃. Similarly, an alkylene group such as n-butylene may be interrupted by the heterogroup N(R″) as follows: —CH₂N(R″)CH₂CH₂CH₂—, —CH₂CH₂N(R″)CH₂CH₂—, or —CH₂CH₂CH₂N(R″)CH₂—. Typically an interrupted group, for instance an interrupted C₁₋₂₀ alkylene or C₁₋₂₀ alkyl group, is interrupted by 1, 2 or 3 heteroatoms or heterogroups or by 1, 2 or 3 arylene (typically phenylene) groups. More typically, an interrupted group, for instance an interrupted C₁₋₂₀ alkylene or C₁₋₂₀ alkyl group, is interrupted by 1 or 2 heteroatoms or heterogroups or by 1 or 2 arylene (typically phenylene) groups. For instance, a C₁₋₂₀ alkyl group such as n-butyl may be interrupted by 2 heterogroups N(R″) as follows: —CH₂N(R″)CH₂N(R″)CH₂CH₃.

An arylene group is an unsubstituted or substituted bidentate moiety obtained by removing two hydrogen atoms, one from each of two different aromatic ring atoms of an aromatic compound, which moiety has from 5 to 14 ring atoms (unless otherwise specified). Typically, each ring has from 5 to 7 or from 5 to 6 ring atoms. An arylene group may be unsubstituted or substituted, for instance, as specified above for aryl.

In this context, the prefixes (e.g., C₅₋₂₀, C₆₋₂₀, C₅₋₁₄, C₅₋₇, C₅₋₆, etc.) denote the number of ring atoms, or range of number of ring atoms, whether carbon atoms or heteroatoms. For example, the term “C₅₋₆ arylene,” as used herein, pertains to an arylene group having 5 or 6 ring atoms. Examples of groups of arylene groups include C₅₋₂₀ arylene, C₆₋₂₀ arylene, C₅₋₁₄ arylene, C₆₋₁₄ arylene, C₆₋₁₀ arylene, C₅₋₁₂ arylene, C₅₋₁₀ arylene, C₅₋₇ arylene, C₅₋₆ arylene, C₅ arylene, and C₆ arylene.

The ring atoms may be all carbon atoms, as in “carboarylene groups” (e.g., C₆₋₂₀ carboarylene, C₆₋₁₄ carboarylene or C₆₋₁₀ carboarylene).

Examples of C₆₋₂₀ arylene groups which do not have ring heteroatoms (i.e., C₆₋₂₀ carboarylene groups) include, but are not limited to, those derived from the compounds discussed above in regard to aryl groups, e.g. phenylene, and also include those derived from aryl groups which are bonded together, e.g. phenylene-phenylene (diphenylene) and phenylene-phenylene-phenylene (triphenylene).

Alternatively, the ring atoms may include one or more heteroatoms, as in “heteroarylene groups” (e.g., C₅₋₁₀ heteroarylene).

Examples of C₅₋₁₀ heteroarylene groups include, but are not limited to, those derived from the compounds discussed above in regard to heteroaryl groups.

As used herein the term oxo represents a group of formula: ═O

As used herein the term acyl represents a group of formula: —C(═O)R, wherein R is an acyl substituent, for example, a substituted or unsubstituted C₁₋₂₀ alkyl group, a substituted or unsubstituted C₃₋₂₀ heterocyclyl group, or a substituted or unsubstituted aryl group. Examples of acyl groups include, but are not limited to, —C(═O)CH₃ (acetyl), —C(═O)CH₂CH₃ (propionyl), —C(═O)C(CH₃)₃ (t-butyryl), and —C(═O)Ph (benzoyl, phenone).

As used herein the term acyloxy (or reverse ester) represents a group of formula: —OC(═O)R, wherein R is an acyloxy substituent, for example, substituted or unsubstituted C₁₋₂₀ alkyl group, a substituted or unsubstituted C₃₋₂₀heterocyclyl group, or a substituted or unsubstituted aryl group, typically a C₁₋₆ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

As used herein the term ester (or carboxylate, carboxylic acid ester or oxycarbonyl) represents a group of formula: —C(═O)OR, wherein R is an ester substituent, for example, a substituted or unsubstituted C₁₋₂₀ alkyl group, a substituted or unsubstituted C₃₋₂₀ heterocyclyl group, or a substituted or unsubstituted aryl group (typically a phenyl group). Examples of ester groups include, but are not limited to, —C(═O)OCH₃, —C(═O)OCH₂CH₃, —C(═O)OC(CH₃)₃, and —C(═O)OPh.

As used herein the term phosphonic acid represents a group of the formula: —P(═O)(OH)₂. As would be understood by the skilled person, a phosphonic acid group can exist in protonated and deprotonated forms (i.e. —P(═O)(OH)₂, —P(═O)(O⁻)₂ and —P(═O)(OH)(O⁻)) all of which are within the scope of the term “phosphonic acid”.

As used herein the term phosphonic acid salt represents a group which is a salt of a phosphonic acid group. For example a phosphonic acid salt may be a group of the formula —P(═O)(OH)(O⁻X⁺) wherein X is a monovalent cation. X⁺ may be an alkali metal cation. X⁺ may be Na⁺ or K⁺, for example.

As used herein the term phosphonate ester represents a group of one of the formulae:

—P(═O)(OR)₂ and —P(═O)(OR)O— wherein each R is independently a phosphonate ester substituent, for example, —H, substituted or unsubstituted C₁₋₂₀ alkyl, substituted or unsubstituted C₃₋₂₀ heterocyclyl, C₃₋₂₀ heterocyclyl substituted with a further C₃₋₂₀ heterocyclyl, substituted or unsubstituted C₁₋₂₀ alkylene-C₃₋₂₀ heterocyclyl, substituted or unsubstituted C₃₋₂₅ cycloalkyl, substituted or unsubstituted C₁₋₂₀ alkylene-C₃₋₂₅ cycloalkyl, aryl, substituted or unsubstituted C₁₋₂₀ alkylene-aryl. Examples of phosphonate ester groups include, but are not limited to, —P(═O)(OCH₃)₂, —P(═O)(OCH₂CH₃)₂, —P(═O)(O-t-Bu)₂, and —P(═O)(OPh)₂,

As used herein the term phosphoric acid represents a group of the formula: —OP(═O)(OH)₂.

As used herein the term phosphate ester represents a group of one of the formulae: —OP(═O)(OR)₂ and —OP(═O)(OR)O⁻ wherein each R is independently a phosphate ester substituent, for example, —H, substituted or unsubstituted C₁₋₂₀ alkyl, substituted or unsubstituted C₃₋₂₀ heterocyclyl, C₃₋₂₀ heterocyclyl substituted with a further C₃₋₂₀ heterocyclyl, substituted or unsubstituted C₁₋₂₀ alkylene-C₃₋₂₀ heterocyclyl, substituted or unsubstituted C₃₋₂₅ cycloalkyl, substituted or unsubstituted C₁₋₂₀ alkylene-C₃₋₂₅ cycloalkyl, aryl, substituted or unsubstituted C₁₋₂₀ alkylene-aryl. Examples of phosphate ester groups include, but are not limited to, —OP(═O)(OCH₃)₂, —OP(═O)(OCH₂CH₃)₂, —OP(═O)(O-t-Bu)₂, and —OP(═O)(OPh)₂.

As used herein the term amino represents a group of formula —NH₂. The term C₁-C₁₀ alkylamino represents a group of formula —NHR′ wherein R′ is a C₁₀ alkyl group, preferably a C₁₋₆ alkyl group, as defined previously. The term di(C₁₋₁₀)alkylamino represents a group of formula —NR′R″ wherein R′ and R″ are the same or different and represent C₁₋₁₀ alkyl groups, preferably C₁₋₆ alkyl groups, as defined previously. The term arylamino represents a group of formula —NHR′ wherein R′ is an aryl group, preferably a phenyl group, as defined previously. The term diarylamino represents a group of formula —NR′R″ wherein R′ and R″ are the same or different and represent aryl groups, preferably phenyl groups, as defined previously. The term arylalkylamino represents a group of formula —NR′R″ wherein R′ is a C₁₋₁₀ alkyl group, preferably a C₁₋₆ alkyl group, and R″ is an aryl group, preferably a phenyl group.

As used herein the term amido represents a group of formula: —C(═O)NR′R″, wherein R′ and R″ are independently amino substituents, as defined for di(C₁₋₁₀)alkylamino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R′ and R″, together with the nitrogen atom to which they are attached, form a heterocyclic structure as in, for example, piperidinocarbonyl, morpholinocarbonyl, thiomorpholinocarbonyl, and piperazinocarbonyl.

As used herein the term acylamido represents a group of formula: —NR¹C(═O)R², wherein R¹ is an amide substituent, for example, hydrogen, a C₁₋₂₀alkyl group, a C₃₋₂₀ heterocyclyl group, an aryl group, preferably hydrogen or a C₁₋₂₀ alkyl group, and R² is an acyl substituent, for example, a C₁₋₂₀ alkyl group, a C₃₋₂₀ heterocyclyl group, or an aryl group, preferably hydrogen or a C₁₋₂₀ alkyl group. Examples of acylamide groups include, but are not limited to, —NHC(═O)CH₃, —NHC(═O)CH₂CH₃, —NHC(═O)Ph, —NHC(═O)C₁₅H₃₁ and —NHC(═O)C₉H₁₉. Thus, a substituted C₁₋₂₀ alkyl group may comprise an acylamido substituent defined by the formula —NHC(═O)—C₁₋₂₀ alkyl, such as —NHC(═O)C₁₅H₃₁ or —NHC(═O)C₉H₁₉. R¹ and R² may together form a cyclic structure, as in, for example, succinimidyl, maleimidyl, and phthalimidyl:

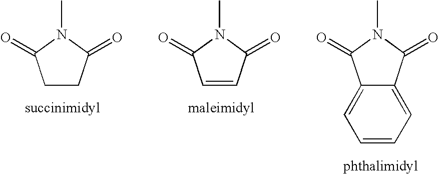

A C₁₋₁₀ alkylthio group is a said C₁₋₁₀ alkyl group, preferably a C₁₋₆ alkyl group, attached to a thio group. An arylthio group is an aryl group, preferably a phenyl group, attached to a thio group.

A C₁₋₂₀ alkoxy group is a said substituted or unsubstituted C₁₋₂₀ alkyl group attached to an oxygen atom. A C₁₋₆ alkoxy group is a said substituted or unsubstituted C₁₋₆ alkyl group attached to an oxygen atom. A C₁₋₄ alkoxy group is a substituted or unsubstituted C₁₋₄ alkyl group attached to an oxygen atom. Said C₁₋₂₀, C₁₋₆ and C₁₋₄ alkyl groups are optionally interrupted as defined herein. Examples of C₁₋₄ alkoxy groups include, —OMe (methoxy), —OEt (ethoxy), —O(nPr) (n-propoxy), —O(iPr) (isopropoxy), —O(nBu) (n-butoxy), —O(sBu) (sec-butoxy), —O(iBu) (isobutoxy), and —O(tBu) (tert-butoxy). Further examples of C₁₋₂₀ alkoxy groups are —O(Adamantyl), —O—CH₂-Adamantyl and —O—CH₂—CH₂-Adamantyl. An aryloxy group is a substituted or unsubstituted aryl group, as defined herein, attached to an oxygen atom. An example of an aryloxy group is —OPh (phenoxy).

Unless otherwise specified, included in the above are the well known ionic, salt, solvate, and protected forms of these substituents. For example, a reference to carboxylic acid or carboxyl group (—COOH) also includes the anionic (carboxylate) form (—COO⁻), a salt or solvate thereof, as well as conventional protected forms. Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms.

Certain compounds may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anti-clinical-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto, enol, and enolate forms, as in, for example, the following tautomeric pairs: keto/enol (illustrated below), imine/enamine, amide/imino alcohol, amidine/amidine, nitroso/oxime, thioketone/enethiol, N-nitroso/hyroxyazo, and nitro/aci-nitro.

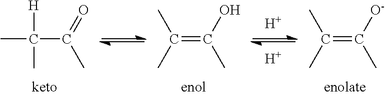

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; 0 may be in any isotopic form, including 160 and 180; and the like.

Unless otherwise specified, a reference to a particular compound includes all such isomeric forms, including (wholly or partially) racemic and other mixtures thereof. Methods for the preparation (e.g., asymmetric synthesis) and separation (e.g., fractional crystallisation and chromatographic means) of such isomeric forms are either known in the art or are readily obtained by adapting known methods, in a known manner.

Unless otherwise specified, a reference to a particular compound also includes ionic, salt, solvate, protected forms and prodrugs thereof.

Examples of pharmaceutically acceptable salts of the compounds for use in accordance with the present invention include salts with inorganic acids such as hydrochloric acid, hydrobromic acid, hydroiodic acid, sulphuric acid, nitric acid and phosphoric acid; and organic acids such as methanesulfonic acid, benzenesulphonic acid, formic acid, acetic acid, trifluoroacetic acid, propionic acid, butyric acid, isobutyric acid, oxalic acid, malonic acid, succinic acid, fumaric acid, maleic acid, lactic acid, malic acid, tartaric acid, citric acid, ethanesulfonic acid, aspartic acid, benzoic acid and glutamic acid. Typically the salt is a hydrochloride, an acetate, a propionate, a benzoate, a butyrate or an isobutyrate. Examples of pharmaceutically acceptable salts are discussed in Berge et al., 1977, “Pharmaceutically Acceptable Salts,” J. Pharm. Sci., Vol. 66, pp. 1-19.

A prodrug of an inhibitor of glycolipid biosynthesis is a compound which, when metabolised (e.g., in vivo), yields the desired active compound. Typically, the prodrug is inactive, or less active than the active compound, but may provide advantageous handling, administration, or metabolic properties.

For example, some prodrugs are O-acylated (acyloxy) derivatives of the active compound, i.e. physiologically acceptable metabolically labile acylated derivatives. During metabolism, the one or more —O-acyl(acyloxy) groups (—O—C(═O)R^{p}) are cleaved to yield the active drug. R^{p} may be a C₁₋₁₀ alkyl group, an aryl group or a C₃₋₂₀ cycloalkyl group. Typically, R^{p} is a C₁₋₁₀ alkyl group including, but not limited to, methyl, ethyl, propyl, butyl, pentyl, hexyl, heptyl, octyl, nonyl and decyl. Such derivatives may be formed by acylation, for example, of any of the hydroxyl groups (—OH) in the parent compound, with, where appropriate, prior protection of any other reactive groups present in the parent compound, followed by deprotection if required. Thus, the free hydroxyl groups on an iminosugar inhibitor of glycolipid biosynthesis (for instance DNJ, DGJ, or an N-alkylated derivative of DNJ or DGJ such as NB-DNJ or NB-DGJ) may be acylated with up to four, typically exactly four, O-acyl groups. The O-acyl groups are enzymatically removed in vivo to provide the non-O-acylated (i.e. hydroxyl-containing) active inhibitor of glycolipid biosynthesis.

Some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

The compound for use in accordance with the invention can be used in the free form or the salt form. For example, when the compound is an iminosugar such as DNJ, DGJ or an N-alkylated derivative thereof, it can be used in the free amine form or in the salt form. The compound may also be used in prodrug form. The prodrug can itself be used in the free form or the salt form. For example, when the prodrug is an iminosugar such as an O-acylated prodrug of DNJ, DGJ or an N-alkylated derivative thereof, it can be used in the free amine form or in the salt form.

In one embodiment the invention provides the use, in the manufacture of a medicament for the treatment of a glycolipid-mediated autoimmune disease, of a compound of the following formula (I):

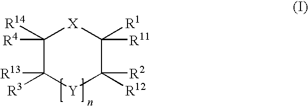

wherein:

X is O, S or NR⁵;

R⁵ is hydrogen, substituted or unsubstituted C₁₋₂₀ alkyl, substituted or unsubstituted C₁₋₂₀ alkylene-aryl, substituted or unsubstituted C₁₋₂₀ alkylene-C₃₋₂₀ heteroaryl, substituted or unsubstituted C₁₋₂₀ alkylene-C₃₋₂₅ cycloalkyl, substituted or unsubstituted C₁₋₂₀ alkylene-C₃₋₂₀ heterocyclyl, substituted or unsubstituted C₁₋₂₀ alkylene-O—C₃₋₂₀ heterocyclyl, substituted or unsubstituted aryl, substituted or unsubstituted C₃₋₂₀ heteroaryl, substituted or unsubstituted C₃₋₂₅ cycloalkyl or substituted or unsubstituted C₃₋₂₀ heterocyclyl, or R⁵ forms, together with R¹, R¹¹, R⁴ or R¹⁴, a substituted or unsubstituted C₁₋₆ alkylene group, wherein said C₁₋₂₀ alkyl and C₁₋₂₀ alkylene are optionally interrupted by N(R′), O, S or arylene wherein R′ is H, C₁₋₆ alkyl or aryl;

n is 0 or 1;

Y is O, S or CR⁶R¹⁶;

R¹, R¹¹, R⁴ and R¹⁴, which may be the same or different, are independently selected from hydrogen, hydroxyl, carboxyl, amino, thiol, substituted or unsubstituted C₁₋₂₀ alkyl, substituted or unsubstituted C₁₋₂₀ alkoxy, substituted or unsubstituted aryloxy, acyl, ester, acyloxy, C₁₋₁₀ alkylamino, di(C₁₋₁₀)alkylamino, amido, acylamido, —O—C₃₋₂₅ cycloalkyl and —O—C₃₋₂₀ heterocyclyl, provided that one of R¹, R¹¹, R⁴ and R¹⁴ may form, together with R⁵, a substituted or unsubstituted C₁₋₆ alkylene group, wherein said C₁₋₂₀ alkyl is optionally interrupted by N(R′), O, S or arylene; and

R², R¹², R³, R¹³, R⁶ and R¹⁶, which may be the same or different, are independently selected from hydrogen, hydroxyl, carboxyl, amino, thiol, substituted or unsubstituted C₁₋₂₀ alkyl, substituted or unsubstituted C₁₋₂₀ alkoxy, substituted or unsubstituted aryloxy, acyl, ester, acyloxy, C₁₋₁₀ alkylamino, di(C₁₋₁₀)alkylamino, amido, acylamido —O—C₃₋₂₅ cycloalkyl and —O—C₃₋₂₀ heterocyclyl, wherein said C₁₋₂₀ alkyl is optionally interrupted by N(R′), O, S or arylene,

or a pharmaceutically acceptable salt thereof.

In the compounds of formula (I), typically either R¹ or R¹¹ (more typically R¹¹) is H. Typically, either R² or R¹² (more typically R¹²) is H. Typically, either R³ or R¹³ (more typically R¹³) is H. Typically, either R⁴ or R¹⁴ (more typically R¹⁴) is H. Typically, where Y is CR⁶R⁶, either R⁶ or R¹⁶ (more typically R¹⁶) is H.

Typically, R¹ or R¹¹ is selected from hydrogen, hydroxyl, carboxyl, substituted or unsubstituted C₁₋₂₀ alkyl, substituted C₁₋₂₀ alkoxy, substituted or unsubstituted aryloxy, —O—C₃₋₂₅ cycloalkyl and —O—C₃₋₂₀ heterocyclyl. More typically, R¹¹ is hydrogen and R¹ is selected from hydrogen, hydroxyl, carboxyl, substituted or unsubstituted C₁₋₂₀ alkyl, substituted C₁₋₂₀ alkoxy, substituted or unsubstituted aryloxy, —O—C₃₋₂₅ cycloalkyl and —O—C₃₋₂₀ heterocyclyl.

Typically, when R¹ or R¹¹ is C₁₋₂₀ alkoxy, said C₁₋₂₀ alkoxy group is substituted with an ester group or an aryl group, for instance with —C(O)OCH₃ or Ph.

Typically, when R¹ or R¹¹ is an aryloxy group, the aryl group bonded to the oxygen of said aryloxy is either substituted or unsubstituted phenyl, or substituted or unsubstituted naphthyl. Typically, the phenyl or naphthyl is either unsubstituted or monosubstituted with halo or methoxy.

When R¹ or R¹¹ is a substituted C₁₋₂₀ alkyl group, the substituent may be a hydroxyl, phosphate ester or phosphonate ester group. For instance, R¹ or R¹¹ may be CH₂OH or a group of the following formula (VII):

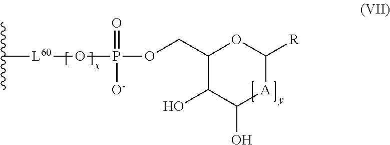

wherein L⁶⁰ is substituted or unsubstituted C₁₋₂₀ alkylene; x is 0 or 1; y is 0 or 1; A is CHR′″ and R is H, C₁₋₂₀ alkyl, C₃₋₂₀ heterocyclyl, C₃₋₂₅ cycloalkyl, aryl or C₁₋₂₀ alkoxy, wherein R′″ is hydroxyl, C₁₋₆ alkoxy, aryloxy or acyl. Typically R′″ is hydroxyl. Typically R is either —OCH₃ or a heterocyclic group of the following structure:

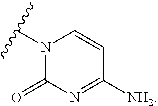

Typically, both R¹ and R¹¹ are groups of formula (VI) above. An example of a compound in which both R¹ and R¹¹ are groups of formula (VII) is cytidin-5′-yl sialylethylphosphonate.

Typically, when R¹ or R¹¹ is unsubstituted C₁₋₂₀ alkyl, it is a methyl, ethyl, propyl, butyl, pentyl, hexyl, heptyl, octyl, nonyl or decyl group.

Typically, when R¹ or R¹¹ is —O—C₃₋₂₅ cycloalkyl, the cycloalkyl group is a group derived from a compound of one of the following formulae, which compound may be substituted or unsubstituted:

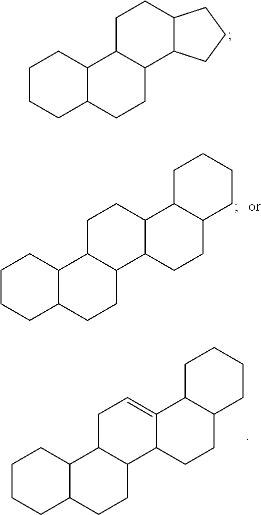

The term “group derived from a compound” in this case means that the group is a monovalent moiety obtained by removing a hydrogen atom from a carbon atom of the compound. An example of a compound in which R¹ or R¹¹ is —O—C₃₋₂₅ cycloalkyl is Soyasaponin I, in which R¹ or R¹¹ has the following structure:

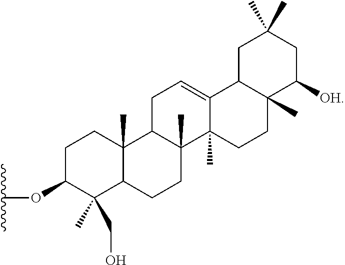

Typically, when R¹ or R¹¹ is —O—C₃₋₂₀ heterocyclyl, said heterocyclyl group is a group derived from a monosaccharide in cyclic form, for instance a group of the following structure:

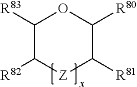

wherein x is 0 or 1; z is CHR⁸⁴; and R⁸⁰, R⁸¹, R⁸², R⁸³ and R⁸⁴, which are the same or different, are independently selected from H, C₁₋₆ alkyl, OH, acyloxy, SH, C₁₋₆ alkoxy, aryloxy, amino, C₁₋₁₀ alkylamino, di(C₁₋₁₀)alkylamino, amido and acylamido. The term “group derived from” in this case means that the group is a monovalent moiety obtained by removing the R⁸⁰, R⁸¹, R⁸², R⁸³ or R⁸⁴ atom from a carbon atom of the above compound.

More typically, when R¹ or R¹¹ is —O—C₃₋₂₀ heterocyclyl, said —O—C₃₋₂₀ heterocyclyl group is a group of any one of the following structures:

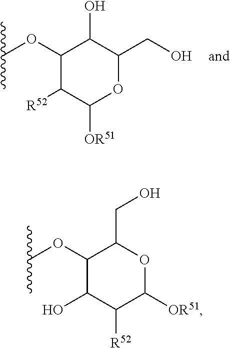

in which R⁵¹ is a substituted or unsubstituted C₁₋₁₀ alkyl group, typically methyl, or a substituted or unsubstituted aryl group, typically a phenyl or naphthyl group. The phenyl or naphthyl may be unsubstituted or substituted. When substituted, the phenyl or naphthyl is typically substituted with a halo group, for instance with a bromo group. R⁵² is typically hydroxyl, C₁₋₁₀ alkoxy, acyloxy, aryloxy or acylamido. Typically, R⁵² is —OH or —NHC(O)Me.

In the compounds of formula (I), typically either R¹ or R¹² is selected from hydrogen, hydroxyl, acyloxy, acylamido, C₁₋₂₀ alkoxy, C₁₋₂₀ alkyl and —O—C₃₋₂₀ heterocyclyl. More typically, R¹² is hydrogen and R² is selected from hydrogen, hydroxyl, acyloxy, C₁₋₂₀ alkoxy, C₁₋₂₀ alkyl and —O—C₃₋₂₀ heterocyclyl.

Typically, when R² or R¹² is acylamido, said acylamido is —NHC(O)CH₃.

Typically, when R² or R¹² is acyloxy, said acyloxy is selected from —OC(O)CH₃, —OC(O)CH₂CH₃, —OC(O)CH₂CH₂CH₃ and —OC(O)CH₂CH₂CH₂CH₃. More typically, when R² or R¹² is acyloxy, said acyloxy is —OC(O)CH₂CH₂CH₃.

Typically, when R² or R¹² is —O—C₃₋₂₀ heterocyclyl, said heterocyclyl group is a group derived from a monosaccharide in cyclic form, for instance a group of the following structure:

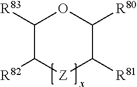

wherein x is 0 or 1; z is CHR⁸⁴; and R⁸⁰, R⁸¹, R⁸², R⁸³ and R⁸⁴, which are the same or different, are independently selected from H, C₁₋₆ alkyl, OH, SH, C₁₋₆ alkoxy, aryloxy, amino, C₁₋₁₀ alkylamino, di(C₁₋₁₀)alkylamino, amido, acylamido and a group derived from a second group of the following structure:

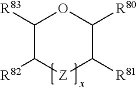

in which second group x is 0 or 1; z is CHR⁸⁴; and R⁸⁰, R⁸¹, R⁸², R⁸³ and R⁸⁴, which are the same or different, are independently selected from H, C₁₋₆ alkyl, OH, SH, C₁₋₆ alkoxy, aryloxy, amino, C₁₋₁₀ alkylamino, di(C₁₋₁₀)alkylamino, amido and acylamido. The term “group derived from” in this case means that the group is a monovalent moiety obtained by removing the R⁸⁰, R⁸¹, R⁸², R⁸³ or R⁸⁴ atom or group from a carbon atom of the compound. Thus when R² or R¹² is —O—C₃₋₂₀ heterocyclyl, said heterocyclyl group may be a group of the following structure:

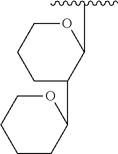

wherein each of the ring carbon atoms is independently unsubstituted or substituted with C₁₋₆ alyl, OH, SH, C₁₋₆ alkoxy, aryloxy, amino, C₁₋₁₀ alkylamino, di(C₁₋₁₀)alkylamino, amido and acylamido. An example of a compound in which R² or R² is —O—C₃₋₂₀ heterocyclyl is Soyasaponin I, in which R² or R¹² is a group of the following structure:

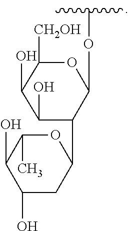

Typically, when R¹ or R¹² is C₁₋₂₀ alkoxy or C₁₋₂₀ alkyl, the group is methyl, ethyl, propyl, butyl, pentyl, hexyl, methoxy, ethoxy, propoxy, butoxy, pentoxy or hexoxy.

More typically, R² or R¹² is selected from H, OH, —OC(O)CH₂CH₂CH₃ and NHC(O)CH₃. In another embodiment, R² or R¹² is selected from H and OH.

In the compounds of formula (I), typically either R³ or R¹³ is selected from hydrogen, hydroxyl, acyloxy, acylamido, C₁₋₂₀ alkoxy and C₁₋₂₀ alkyl. More typically, R¹³ is hydrogen and R³ is selected from hydrogen, hydroxyl, acyloxy, C₁₋₂₀ alkoxy and C₁₋₂₀ alkyl.

Typically, when R³ or R¹³ is acylamido, said acylamido is —NHC(O)CH₃.

Typically, when R³ or R¹³ is acyloxy, said acyloxy is selected from —OC(O)CH₃, —OC(O)CH₂CH₃, —OC(O)CH₂CH₂CH₃ and —OC(O)CH₂CH₂CH₂CH₃. More typically, when R³ or R¹³ is acyloxy, said acyloxy is —OC(O)CH₂CH₂CH₃.

Typically, when R³ or R¹³ is C₁₋₂₀ alkoxy or C₁₋₂₀ alkyl, the group is methyl, ethyl, propyl, butyl, pentyl, hexyl, methoxy, ethoxy, propoxy, butoxy, pentoxy or hexoxy.

More typically, R³ or R¹³ is selected from H, OH and NHC(O)CH₃. In another embodiment, R³ or R¹³ is selected from H and OH.

In the compounds of formula (I), typically either R⁴ or R¹⁴ is hydrogen, hydroxyl, acyloxy, carboxyl, ester or C₁₋₂₀ alkyl which is substituted or unsubstituted, or R⁴ or R¹⁴ forms, together with R⁵, a substituted or unsubstituted C₁₋₆ alkylene group. More typically, R¹⁴ is hydrogen and R⁴ is hydrogen, hydroxyl, acyloxy, carboxyl, ester or C₁₋₂₀ alkyl which is substituted or unsubstituted, or R⁴ forms, together with R⁵, a substituted or unsubstituted C₁₋₆ alkylene group.

Typically, when R⁴ or R¹⁴ is acyloxy, said acyloxy is selected from —OC(O)CH₃, —OC(O)CH₂CH₃, —OC(O)CH₂CH₂CH₃ and —OC(O)CH₂CH₂CH₂CH₃.

Typically, when R⁴ or R¹⁴ is a C₁₋₂₀ alkyl, said C₁₋₂₀ alkyl is substituted with one, two, three or four groups selected from hydroxyl, acyloxy, thiol and —SC(O)R⁹⁵, wherein R⁹⁵ is C₁₋₆ alkyl. More typically, said C₁₋₂₀ alkyl is methyl, ethyl, propyl or butyl substituted with one, two, three or four groups respectively, which groups are selected from hydroxyl, acyloxy and thiol, more typically from hydroxyl and thiol.

Typically, when R⁴ or R¹⁴ forms, together with R⁵, a substituted or unsubstituted C₁₋₆ alkylene group, said alkylene group is substituted or unsubstituted propylene. Typically, said propylene is unsubstituted or substituted with a C₁₋₄ alkyl group, for instance with a methyl group. Examples of compounds of formula (I) in which R⁴ or R¹⁴ forms, together with R⁵, a methyl-substituted propylene group are Castanospermine and MDL25874, whose structures are given below.

R⁴ or R¹⁴ is typically H, —CH₂OH, —CH₂SH, —CH(OH)CH(OH)CH₂OH or —COOH or R⁴ or R¹⁴ forms, together with R⁵, a propylene group substituted with a methyl group.

In the compounds of formula (I), typically n is 1, Y is CR⁶R¹⁶ and either R¹ or R¹⁶ is selected from hydrogen, hydroxyl, acyloxy, amino, C₁₋₂₀ alkoxy and C₁₋₂₀ alkyl. More typically, n is 1, Y is CR⁶R¹⁶, R¹⁶ is hydrogen and R⁶ is selected from hydrogen, hydroxyl, acyloxy, amino, C₁₋₂₀ alkoxy and C₁₋₂₀ alkyl.

Typically, when R⁶ or R¹⁶ is acyloxy, said acyloxy is selected from —OC(O)CH₃, —OC(O)CH₂CH₃, —OC(O)CH₂CH₂CH₃ and —OC(O)CH₂CH₂CH₂CH₃.

Typically, when R⁶ or R¹⁶ is C₁₋₂₀ alkoxy or C₁₋₂₀ alkyl, the group is methyl, ethyl, propyl, butyl, pentyl, hexyl, methoxy, ethoxy, propoxy, butoxy, pentoxy or hexoxy.

Typically, R⁶ or R¹⁶ is selected from —OH and —NH₂.

Alternatively, n is 1 and Y is O or S. More typically, n is 1 and Y is O.

Alternatively, n is 0.

In the compounds of formula (I), typically R⁵ is hydrogen, substituted or unsubstituted C₁₋₂₀ alkyl, substituted or unsubstituted C₁₋₂₀ alkylene-aryl, substituted or unsubstituted C₁₋₂₀ alkylene-C₃₋₂₀ heteroaryl, substituted or unsubstituted C₁₋₂₀ alkylene-C₃₋₂₅ cycloalkyl, substituted or unsubstituted C₁₋₂₀ alkylene-C₃₋₂₀ heterocyclyl, substituted or unsubstituted C₁₋₂₀ alkylene-O—C₃₋₂₀ heterocyclyl or R⁵ forms, together with R¹, R¹¹, R⁴ or R¹⁴, a substituted or unsubstituted C₁₋₆ alkylene group, wherein said C₁₋₂₀ alkyl and C₁₋₂₀ alkylene are optionally interrupted by N(R′), O, S or arylene wherein R′ is H, C₁₋₆ alkyl or aryl.

Typically, when R⁵ is substituted or unsubstituted C₁₋₂₀ alkylene-O—C₃₋₂₀ heterocyclyl, said C₁₋₂₀ alkylene is unsubstituted, and is, for instance, an unsubstituted C₁₋₄ alkylene group, for example ethylene. Typically, when R⁵ is substituted or unsubstituted C₁₋₂₀ alkylene-O—C₃₋₂₀ heterocyclyl, said C₃₋₂₀ heterocyclyl is a group of the following formula (m):

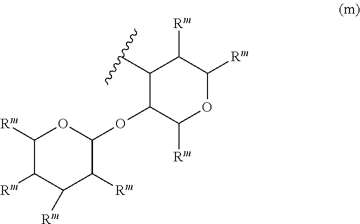

wherein each R^{m}, which is the same or different, is independently selected from C₁₋₆ alkyl, OH, acyloxy, SH, C₁₋₆ alkoxy, aryloxy, amino, C₁₋₁₀ alkylamino, di(C₁₋₁₀)alkylamino, amido and acylamido. More typically, when R⁵ is substituted or unsubstituted C₁₋₂₀ alkylene-O—C₃₋₂₀ heterocyclyl, said C₃₋₂₀ heterocyclyl is a group of the following structure:

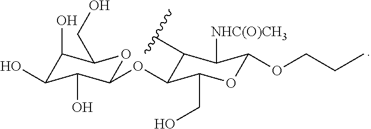

Alternatively, R⁵ may be substituted or unsubstituted aryl, substituted or unsubstituted C₃₋₂₀ heteroaryl, substituted or unsubstituted C₃₋₂₀ heterocyclyl or substituted or unsubstituted C₃₋₂₅ cycloalkyl. Thus, R¹ may be substituted or unsubstituted phenyl or substituted or unsubstituted cyclohexyl, for example.

In the compounds of formula (I), typically X is NR⁵, and R⁵ forms, together with R⁴ or R¹⁴ (typically R⁴), a substituted or unsubstituted C₁₋₆ alkylene group, or R⁵ is selected from hydrogen, unsubstituted or substituted C₁₋₂₀ alkyl which is optionally interrupted by 0, and a group of the following formula (VIII)

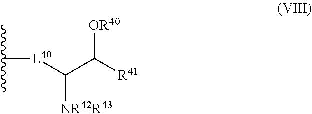

in which:

R⁴⁰ and R⁴², which are the same or different, are independently selected from H, substituted or unsubstituted C₁₋₆ alkyl or substituted or unsubstituted phenyl;

R⁴¹ is H, substituted or unsubstituted aryl, —CH═CHR⁴⁴, or substituted or unsubstituted C₁₋₂₀ alkyl, which C₁₋₂₀ alkyl is optionally interrupted by N(R′), O, S or arylene wherein R′ is H, C₁₋₆ alkyl or aryl;

R⁴³ is H, substituted or unsubstituted C₁₋₆ alkyl, substituted or unsubstituted phenyl or —C(O)R⁴⁷;

R⁴⁴ is H or substituted or unsubstituted C₁₋₂₀ alkyl, which C₁₋₂₀ alkyl is optionally interrupted by N(R′), O, S or arylene;

R⁴⁷ is substituted or unsubstituted C₁₋₂₀ alkyl, which C₁₋₂₀ alkyl is optionally interrupted by N(R′), O, S or arylene; and

L⁴⁰ is substituted or unsubstituted C₁₋₁₀ alkylene.

Typically, R⁴⁰ is H. Typically, R⁴² is H. Typically, R⁴³ is H or —C(O)R⁴⁷. More typically, R⁴³ is —C(O)R⁴⁷. Typically, R⁴⁷ is unsubstituted C₁₋₂₀ alkyl. R⁴⁷ may be, for instance, C₉H₁₉ or C₁₅H₃₁. Typically L⁴⁰ is CH₂. In one embodiment, R⁴¹ is —CH═CHR⁴⁴ and R⁴⁴ is unsubstituted C₁₋₂₀ alkyl. In that embodiment, R⁴⁴ may be, for instance, —C₁₃H₂₇. In another embodiment, R⁴¹ is a group of the following formula (VIIIa):

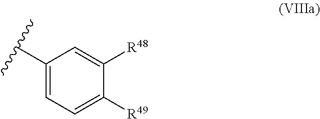

in which R⁴⁸ is H, C₁₋₆ alkyl, phenyl or, together with R⁴⁹ a bidentate group of the structure —O-alk-O—; R⁴⁹ is H, C₁₋₆ alkyl, phenyl or, together with R⁴⁸ a bidentate group of the structure —O-alk-O—, wherein alk is substituted or unsubstituted C₁₋₆ alkylene. Typically, R⁴⁸ is H or, together with R⁴⁹ a bidentate group of the structure —O—CH₂—CH₂—O—. Typically, R⁴⁹ is H, OH or, together with R⁴⁸ a bidenitate group of the structure —O—CH₂—CH₂—O—. Typically, R⁴⁸ is H and R⁴⁹ is either H or OH.

Typically, when R⁵ is C₁₋₂₀ alkyl optionally interrupted by O, R⁵ is methyl, ethyl, propyl, butyl, pentyl, hexyl, heptyl, octyl, nonyl, decyl, methyl-O—R⁹⁰, ethyl-O—R⁹⁰, propyl-O—R⁹⁰, butyl-O—R⁹⁰, pentyl-O—R⁹⁰, hexyl-O—R⁹⁰, heptyl-O—R⁹⁰, octyl-O—R⁹⁰, nonyl-O—R⁹⁰ or decyl-O—R⁹⁰ wherein R⁹⁰ is methyl, ethyl, propyl, butyl, pentyl, hexyl, heptyl, octyl, nonyl, decyl or adamantyl.

Typically, when R⁵ forms, together with R⁴ or R¹⁴, a substituted or unsubstituted C₁₋₆ alkylene group, the alkylene group is substituted or unsubstituted propylene. Typically, said propylene is unsubstituted or substituted with a C₁₋₄ alkyl group, for instance with a methyl group. Examples of compounds of formula (I) in which R⁴ or R¹⁴ forms, together with R⁵, a methyl-substituted propylene group are Castanospermine and MDL25874, whose structures are given below.

Alternatively, X is O or S. More typically, X is O.

Typically, R², R³, R¹⁴ and R¹⁶ are all H and the compound is of formula (Ia) below:

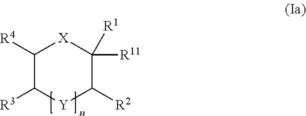

wherein X is O, S or NR⁵ Y is O, S or CHR⁶; n is 0 or 1; and R¹, R², R³, R⁴, R⁵, R⁶ and R¹¹ are as defined above for formula (I).

In one embodiment, the compound is of formula (Ia) and: X is NR⁵; n is 1; Y is CHR⁶; and R⁵ is selected from: hydrogen and unsubstituted or substituted C₁₋₂₀ alkyl which is optionally interrupted by 0, or R⁵ forms, together with R⁴, a substituted or unsubstituted C₁₋₆ alkylene group.

Typically in this embodiment, R¹ is H. Typically in this embodiment, R¹, R², R³ and R⁶, which may be the same or different, are independently selected from H, OH, acyloxy, and substituted or unsubstituted C₁₋₆ alkyl. When said C₁₋₆ alkyl is substituted, it is typically substituted with 1, 2, 3 or 4 groups selected from hydroxyl and acyloxy. Typically, in this embodiment, R⁴ is either C₁₋₆ alkyl substituted with 1, 2, 3 or 4 groups selected from hydroxyl and acyloxy, or R⁴ forms, together with R⁵, a substituted or unsubstituted C₁₋₆ alkylene group. For instance, R⁴ may be methyl, ethyl, propyl or butyl substituted with 1, 2, 3 or 4 groups respectively, which groups are selected from hydroxyl and acyloxy, more typically hydroxyl. R⁴ may be CH₂OH. Alternatively R⁴ may be a group which, together with R⁵, is a substituted or unsubstituted propylene group. Typically, in this embodiment, R², R³ and R⁶ are all OH. Typically, in this embodiment R¹ is selected from H, OH and C₁₋₆ alkyl which is unsubstituted or substituted with one, two, three or four groups selected from hydroxyl and acyloxy. For instance, R¹ may be H, OH, unsubstituted C₁₋₆ alkyl, methyl, ethyl, propyl or butyl, which methyl, ethyl, propyl and butyl are substituted with 1, 2, 3 or 4 groups respectively, which groups are selected from hydroxyl and acyloxy, more typically hydroxyl. More typically, R⁴ is H, OH, CH₂OH or C₁₋₆ alkyl. In this embodiment, the modification of the iminosugar core with an N-alkyl chain such as a N-butyl group (as in NB-DNJ) or a N-nonyl group (as in N,N-DNJ) is believed to be important for clinical applications. Thus, typically in this embodiment R⁵ is C₁₋₂₀ alkyl which is optionally interrupted by O. For instance, R⁵ may be methyl, ethyl, propyl, butyl, pentyl, hexyl, heptyl, octyl, nonyl, decyl, methyl-O—R⁹⁰, ethyl-O—R⁹⁰, propyl-O—R⁹⁰, butyl-O—R⁹⁰, pentyl-O—R⁹⁰, hexyl-O—R⁹⁰, heptyl-O—R⁹⁰, octyl-O—R⁹⁰, nonyl-O—R⁹⁰ or decyl-O—R⁹⁰ wherein R⁹⁰ is methyl, ethyl, propyl, butyl, pentyl, hexyl, heptyl, octyl, nonyl, decyl or adamantyl. Alternatively, R⁵, together with R⁴ is a substituted or unsubstituted C₁₋₆ alkylene group. Typically, the alkylene group is substituted or unsubstituted propylene. Typically, said propylene is unsubstituted or substituted with a C₁₋₄ alkyl group, for instance with a methyl group. Alternatively, R⁵ may be H. Typically, when R² is acyloxy, said acyloxy is selected from —OC(O)CH₃, —OC(O)CH₂CH₃, —OC(O)CH₂CH₁₂CH₃ and —OC(O)CH₂CH₂CH₂CH₃. More typically, when R² is acyloxy, said acyloxy is —OC(O)CH₂CH₂CH₃. Alternatively, R⁵ is substituted or unsubstituted C₁₋₂₀ alkylene-O—C₃₋₂₀ heterocyclyl. More typically, R⁵ is C₁₋₄ alkylene-O—C₃₋₂₀ heterocyclyl, wherein said C₁₋₄ alkylene is unsubstituted and said C₃₋₂₀ heterocyclyl is a group of the following formula (m):

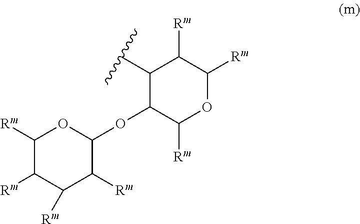

wherein each R^{m}, which are the same or different, is independently selected from C₁₋₆ alkyl, OH, acyloxy, SH, C₁₋₆ alkoxy, aryloxy, amino, C₁₋₁₀ alkylamino, di(C₁₋₁₀)alkylamino, amido and acylamido. Even more typically, R⁵ is C₁₋₄ alkylene-O—C₃₋₂₀ heterocyclyl, wherein said C₁₋₄ alkylene is unsubstituted and said C₃₋₂₀ heterocyclyl is a group of the following structure:

TABLE 4

| Compound | Compound structure (name) |
|---|---|
| 67 | 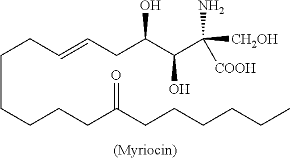 |
| 68 | 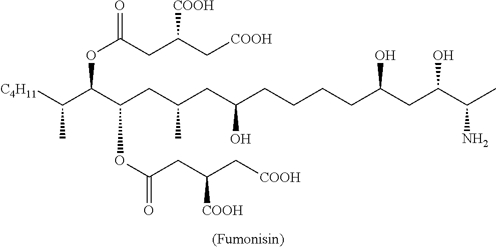 |

In another embodiment, the compound is of formula (Ia) and: X is NR⁵; Y is O or S; n is either 0 or 1; and R⁵ is selected from hydrogen and a group of the following formula (VIII):

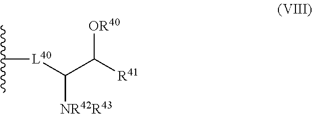

in which R⁴⁰ and R⁴², which are the same or different, are independently selected from H, substituted or unsubstituted C₁₋₆ alkyl or substituted or unsubstituted phenyl; R⁴¹ is H, substituted or unsubstituted aryl, —CH═CHR⁴⁴, or substituted or unsubstituted C₁₋₂₀ alkyl, which C₁₋₂₀ alkyl is optionally interrupted by N(R′), O, S or arylene wherein R′ is H, C₁₋₆ alkyl or aryl; R⁴³ is H, substituted or unsubstituted C₁₋₆ alkyl, substituted or unsubstituted phenyl or —C(O)R⁴⁷; R⁴⁴ is H or substituted or unsubstituted C₁₋₂₀ alkyl, which C₁₋₂₀ alkyl is optionally interrupted by N(R′), O, S or arylene; R⁴⁷ is substituted or unsubstituted C₁₋₂₀ alkyl, which C₁₋₂₀ alkyl is optionally interrupted by N(R′), O, S or arylene; and L⁴⁰ is substituted or unsubstituted C₁₋₁₀ alkylene. Typically, R⁴⁰ is H. Typically, R⁴² is H. Typically, R⁴³ is H or —C(O)R⁴⁷. More typically, R⁴³ is —C(O)R⁴⁷. Typically, R⁴⁷ is unsubstituted C₁₋₂₀ alkyl. R⁴⁷ may be, for instance, C₉H₁₉ or C₅H₃₁. Typically L⁴⁰ is CH₂. In one embodiment, R⁴¹ is —CH═CHR⁴⁴ and R⁴⁴ is unsubstituted C₁₋₂₀ alkyl. In that embodiment, R⁴⁴ may be, for instance, —C₁₃H₂₇. Alternatively, R⁴¹ is a group of the following formula (VIIIa):

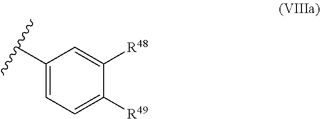

in which R⁴⁵ is H, C₁₋₆ alkyl, phenyl or, together with R⁴⁹ a bidentate group of the structure —O-alk-O—; R⁴⁹ is H, C₁₋₆ alkyl, phenyl or, together with R⁴⁸ a bidentate group of the structure —O-alk-O—, wherein alk is substituted or unsubstituted C₁₋₆ alkylene. Typically,

R⁴⁸ is H or, together with R⁴⁹ a bidentate group of the structure —O—CH₂—CH₂—O—. Typically, R⁴⁹ is H, OH or, together with R⁴⁸ a bidentate group of the structure —O—CH₂—CH₂—O—. Typically, R⁴³ is H and R⁴⁹ is either H or OH. Typically in this embodiment, R¹¹ is H. Typically in this embodiment, R¹, R², R³, R⁴ and R⁶, which may be the same or different, are independently selected from H, OH, acyloxy and C₁₋₆ alkyl which is unsubstituted or substituted with one, two, three or four groups selected from hydroxyl and acyloxy. More typically, in this embodiment, R¹, R², R³, R⁴ and R⁶ are independently selected from H, OH and CH₂OH. Typically, in this embodiment, Y is O. Examples of compounds of this embodiment include D,L-threo-1-phenyl-2-decanoylamino-3-morpholino-1-propanol (PDMP); D,L-threo-1-phenyl-2-hexadecanoylamino-3-morpholino-1-propanol (PPMP); D-threo-1-phenyl-2-palmitoilamino-3-pyrrolidino-1-propanol (P4); 4′-hydroxy-D-threo-1-phenyl-2-palmitoilamino-3-pyrrolidino-1-propanol (4′-hydroxy-P4); 3′,4′-ethylenedioxy-P4 (EtD0-P4); and 2,5-dihydroxymethyl-3,4-dihydroxypyrrolidine (L-DMDP).

In another embodiment, the compound is of formula (Ia) and: X is O or S; n is 1; Y is CHR⁶; R⁶ is H, hydroxyl, acyloxy, C₁₋₂₀ alkoxy, C₁₋₁₀ alkylamino or di(C₁₋₁₀)alkylamino; R¹¹ is H; R² and R³, which may be the same or different, are independently selected from H, hydroxyl, C₁₋₂₀ alkoxy, acyloxy or acylamido; R⁴ is H, hydroxyl, acyloxy, thiol or C₁₋₂₀ alkyl which is unsubstituted or substituted with one, two, three or four groups selected from hydroxyl, acyloxy and thiol; and R¹ is C₁₋₂₀ alkoxy, aryloxy or —O—C₃₋₂₀ heterocyclyl, wherein said heterocyclyl is a group derived from a group of the following structure:

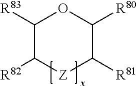

wherein x is 0 or 1; z is CHR⁸⁴; and R⁸⁰, R⁸¹, R⁸², R⁸³ and R⁸⁴, Which are the same or different, are independently selected from H, C₁₋₆ alkyl, OH, acyloxy, SH, C₁₋₆ alkoxy, aryloxy, amino, C₁₋₁₀ alkylamino, di(C₁₋₁₀)alkylamino, amido and acylamido. Typically, in this embodiment, X is O. Examples of compounds of this embodiment are the Galactosyltransferase inhibitor compounds described in Chung S J, Bioorg Med Chem. Lett. 1998 Dec. 1; 8(23):3359-64, whose structures are given hereinbelow.

Typically, in this embodiment, R¹ is a group of any one of the following structures:

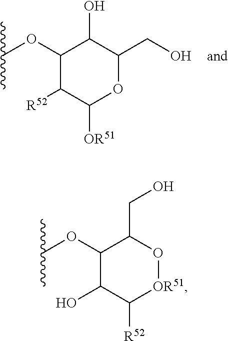

in which R⁵¹ is a substituted or unsubstituted C₁₋₁₀ alkyl group, typically methyl, or a substituted or unsubstituted aryl group, typically a phenyl or naphthyl group. The phenyl or naphthyl may be unsubstituted or substituted. When substituted, the phenyl or naphthyl is typically substituted with a halo group, for instance with a bromo group. R⁵² is typically hydroxyl, C₁₋₁₀ alkoxy, acyloxy, aryloxy or acylamido. Typically, R⁵² is —OH or —NHC(O)Me.

Alternatively, in this embodiment, R¹ may be C₁₋₂₀ alkoxy wherein said C₁₋₂₀ alkoxy group is substituted with an ester group or an aryl group, for instance with —C(O)OCH₃ or Ph. Alternatively, in this embodiment, R¹ may be aryloxy wherein the aryl group bonded to the oxygen of said aryloxy is either substituted or unsubstituted phenyl, or substituted or unsubstituted naphthyl. Typically, the phenyl or naphthyl is either unsubstituted or monosubstituted with halo or methoxy.

Typically, in this embodiment, R⁶ is H, amino or hydroxyl, more typically, amino or hydroxyl. Typically, in this embodiment, R² is H, hydroxyl or —NHC(O)CH₃, more typically hydroxyl or —NHC(O)CH₃. Typically, in this embodiment, R³ is H or hydroxyl, more typically hydroxyl. Typically, in this embodiment, R⁴ is H, CH₂OH or CH₂SH, more typically CH₂OH or CH₂SH.

In another embodiment, the compound is of formula (Ia) and:

X is O or S; n is 1; Y is CHR⁶; R⁶ is H, hydroxyl, acyloxy or C₁₋₂₀ alkoxy;

R¹ and R¹¹ which may be the same or different, are independently selected from H, C₁₋₂₀ alkyl, hydroxyl, acyloxy, C₁₋₂₀ alkoxy, carboxyl, ester, —O—C₃₋₂₅ cycloalkyl, and a group of the following formula (VII):

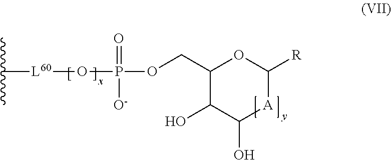

wherein L⁶⁰ is substituted or unsubstituted C₁₋₂₀ alkylene; x is 0 or 1; y is 0 or 1; A is CHR′″ and R is H, C₁₋₂₀ alkyl, C₃₋₂₀ heterocyclyl, C₃₋₂₅ cycloalkyl, aryl or C₁₋₂₀ alkoxy, wherein R′″ is hydroxyl, C₁₋₆ alkoxy, aryloxy or acyl;

R² is H, C₁₋₂₀ alkyl, hydroxyl, acyloxy or —O—C₃₋₂₀ heterocyclyl, wherein said heterocyclyl is a group derived from a group of the following structure:

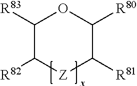

wherein x is 0 or 1; z is CHR⁸⁴; and R⁸⁰, R⁸¹, R⁸², R⁸³ and R⁸⁴, which are the same or different, are independently selected from H, C₁₋₆ alkyl, OH, SH, C₁₋₆ alkoxy, aryloxy, amino, C₁₋₁₀ alkylamino, di(C₁₋₁₀)alkylamino, amido, acylamido and a group derived from a second group of the following structure:

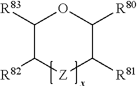

in which second group x is 0 or 1; z is CHR⁸⁴; and R⁸⁰, R⁸¹, R⁸², R⁸³ and R⁸⁴, which are the same or different, are independently selected from H, C₁₋₆ alkyl, OH, SH, C₁₋₆ alkoxy, aryloxy, amino, C₁₋₁₀ alkylamino, di(C₁₋₁₀)alkylamino, amido and acylamido;

R³ is H, hydroxyl, acyloxy, C₁₋₂₀ alkoxy or acylamido; and

R⁴ is H, carboxyl, ester or C₁₋₂₀ alkyl which is unsubstituted or substituted with one, two, three or four groups selected from hydroxyl and thiol.

Examples of compounds of this embodiment are sialic acid, cytidin-5′-yl sialylethylphosphonate and Soyasaponin I.

Typically, in this embodiment, X is O.

Typically, in this embodiment, R⁶ is H or hydroxyl, more typically hydroxyl.

Typically, in this embodiment, R¹ and R¹¹ are independently selected from H, hydroxyl, carboxyl, —O—C₃₋₂₅ cycloalkyl and a group of formula (VI) in which L⁶⁰ is ethylene or methylene, R′″ is hydroxyl, and R is either —OCH₃ or a heterocyclic group of the following structure:

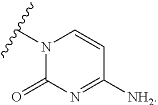

Both R¹ and R¹¹ may be groups of formula (VII).

When R¹ or R¹¹ is —O—C₃₋₂₅ cycloalkyl, the cycloalkyl group is a group derived from a compound of one of the following formulae, which compound may be substituted or unsubstituted:

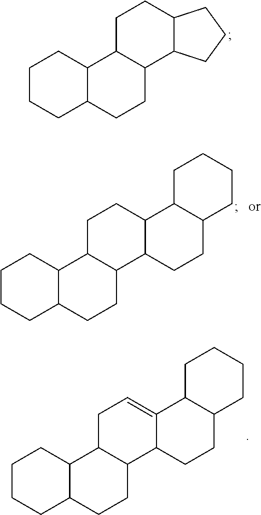

More typically, the cycloalkyl group of said —O—C₃₋₂₅ cycloalkyl is a group derived from the following compound:

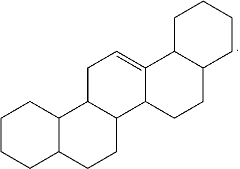

Typically, if either R¹ or R¹¹ is —O—C₃₋₂₅ cycloalkyl, then the other one of those groups, i.e. R¹¹ or R¹ respectively, is H.

Typically, in this embodiment, R² is H or —O—C₃₋₂₀ heterocyclyl, wherein said heterocyclyl is a group of the following structure:

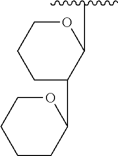

wherein each of the ring carbon atoms is independently unsubstituted or substituted with C₁₋₆ alkyl, OH, SH, C₁₋₆ alkoxy, aryloxy, amino, C₁₋₁₀ alkylamino, di(C₁₋₁₀)alkylamino, amido and acylamido. Typically, each of the ring carbon atoms is independently unsubstituted or substituted with OH, CH₂OH or a C₁₋₆ alkyl group, for instance a methyl group.

Typically, in this embodiment, R³ is hydroxyl or acylamido. More typically, R³ is hydroxyl or NHC(O)CH₃.

Typically, in this embodiment, R⁴ is carboxyl, methyl, ethyl, propyl or butyl, which methyl, ethyl, propyl or butyl are substituted with one, two, three and four groups respectively, which groups are selected from hydroxyl and thiol. More typically, R⁴ is carboxyl or —CH(OH)CH(OH)CH₂OH.

Any one of the following compounds of formula (I) may be employed in the present invention:
Iminosugars (azasugars) such as: N-butyldeoxynojirimycin (NB-DNJ), also known as miglustat or ZAVESCA®; N-nonyldeoxynojirimycin (N,N-DNJ); N-butyldeoxygalactonojirimycin (NB-DGJ); N-5-adamantane-1-yl-methoxypentyl-deoxynojirimycin (AMP-DNJ); alpha-homogalactonojirimycin (HGJ); Nojirimycin (NJ); Deoxynojirimycin (DNJ); N7-oxadecyl-deoxynojirimycin; deoxygalactonojirimycin (DGJ); N-butyl-deoxygalactonojirimycin (NB-DGJ); N-nonyl-deoxygalactonojirimycin (N,N-DGJ); N-nonyl-6deoxygalactonojirimycin; N7-oxanonyl-6deoxy-DGJ; alpha-homoallonojirimycin (HAJ); beta-1-C-butyl-deoxygalactonojirimycin (CB-DGJ). Such compounds are glycosyltransferase inhibitors (“sugar mimics”).
D,L-threo-1-phenyl-2-decanoylamino-3-morpholino-1-propanol (PDMP); D,L-threo-1-phenyl-2-hexadecanoylamino-3-morpholino-1-propanol (PPMP); D-threo-1-phenyl-2-palmitoilamino-3-pyrrolidino-1-propanol (P4); 4′-hydroxy-D-threo-1-phenyl-2-palmitoilamino-3-pyrrolidino-1-propanol (4′-hydroxy-P4); 3′,4′-ethylenedioxy-P4 (EtDO-P4); 2,5-dihydroxymethyl-3,4-dihydroxypyrrolidine (L-DMDP). Such compounds are glycosyltransferase inhibitors and derivatives of sphingosine (“lipid mimics”).
Iminosugars such as Castanospermine and MDL25874, which have the following structures respectively:

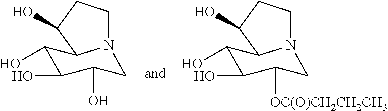

Sialyltransferase inhibitors such as N-acetylneuraminic acid (sialic acid); cytidin-5′-yl sialylethylphosphonate; and Soyasaponin I.
Galactosyltransferase inhibitor compounds of the following structures, which compounds are described in Chung S J, Bioorg Med Chem. Lett. 1998 Dec. 1; 8(23):3359-64:

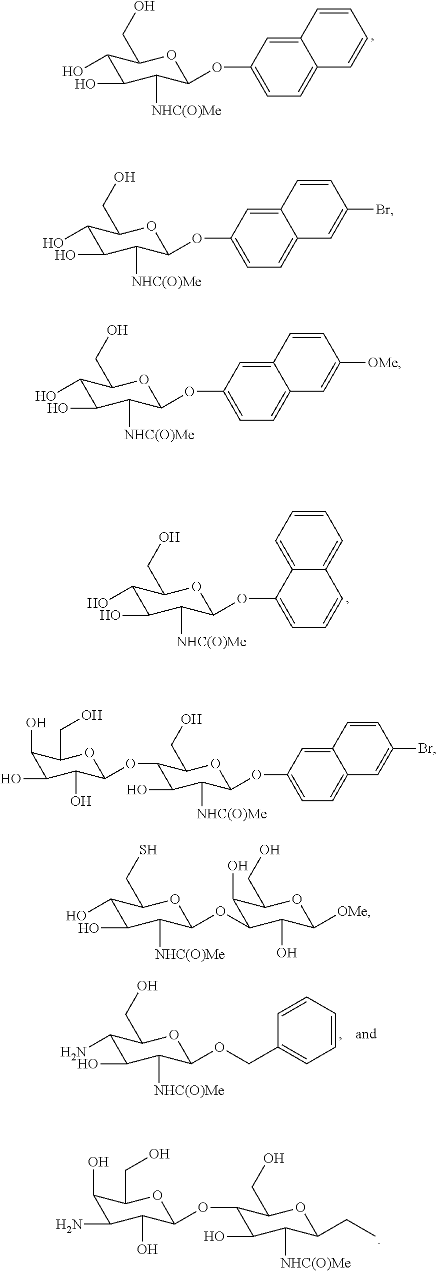

Iminosugars, such as 1,5-dideoxy-1,5-imino-D-glucitol, and their N-alkyl, N-acyl and N-aryl, and optionally O-acylated derivatives, such as: 1,5-(Butylimino)-1,5-dideoxy-D-glucitol, also known as N-butyldeoxynojirimycin (NB-DNJ), miglustat or ZAVESCA®; 1,5-(Methylimino)-1,5-dideoxy-D-glucitol; 1,5-(Hexylimino)-1,5-dideoxy-D-glucitol; 1,5-(Nonylylimino)-1,5-dideoxy-D-glucitol; 1,5-(2-Ethylbutylimino)-1,5-dideoxy-D-glucitol; 1,5-(2-Methylpentylimino)-1,5-dideoxy-D-glucitol; 1,5-(Benzyloxycarbonylimino)-1,5-dideoxy-D-glucitol, tetraacetate; 1,5-(Phenylacetylimino)-1,5-dideoxy-D-glucitol, tetraacetate; 1,5-(Benzoylimino)-1,5-dideoxy-D-glucitol, tetraacetate; 1,5-(Butylimino)-1,5-dideoxy-D-glucitol, tetraacetate; 1,5-(Ethyl malonylimino)-1,5-dideoxy-D-glucitol, tetraacetate; 1,5-(Hexylimino)-1,5-dideoxy-D-glucitol, tetraacetate; 1,5-(Nonylimino)-1,5-dideoxy-D-glucitol, tetraacetate; 1,5-(Benzyloxycarbonylimino)-1,5-dideoxy-D-glucitol, tetraisobutyrate; 1,5-(Butylimino)-1,5-dideoxy-D-glucitol, tetrabutyrate; 1,5-(Butylimino)-1,5-dideoxy-D-glucitol, tetrapropionate; 1,5-(Butylimino)-1,5-dideoxy-D-glucitol, tetrabenzoate; 1,5-Dideoxy-1,5-imino-D-glucitol, tetraisobutyrate; 1,5-(Hydrocinnamoylimino)-1,5-dideoxy-D-glucitol, tetraacetate; 1,5-(Methyl malonylimino)-1,5-dideoxy-D-glucitol, tetraacetate; 1,5-(Butylimino)-1,5-dideoxy-D-glucitol, tetraisobutyrate; 1,5-(Butylimino)-1,5-dideoxy-4R,6-O-(phenylmethylene)-D-glucitol, diacetate; 1,5-[(Phenoxymethyl)carbonylimino]-1,5-dideoxy-D-glucitol, tetraacetate; 1,5-[(Ethylbutyl)imino]-1,5-dideoxy-D-glucitol, tetraacetate; 1,5-(Butylimino)-1,5-dideoxy-D-glucitol, 2,3-diacetate; 1,5-(Hexylimino)-1,5-dideoxy-4R,6-O-(phenylmethylene)-D-glucitol, diacetate; 1,5-(Hexylimino)-1,5-dideoxy-D-glucitol, 2,3-diacetate; 1,5-[(2-Methylpentyl)imino]-1,5-dideoxy-D-glucitol, tetraacetate; 1,5-(Butylimino)-1,5-dideoxy-D-glucitol, 6-acetate; 1,5-[(3-Nicotinoyl)imino]-1,5-dideoxy-D-glucitol, tetraacetate; 1,5-(Cinnamoylimino)-1,5-dideoxy-D-glucitol, tetraacetate; 1,5-(Butylimino)-1,5-dideoxy-D-glucitol, 2,3-dibutyrate; 1,5-(Butylimino)-1,5-dideoxy-4R,6-O-(phenylmethylene)-D-glucitol, 2,3-dibutyrate; 1,5-(Phenylacetylimino)-1,5-dideoxy-D-glucitol, tetraisobutyrate; 1,5-[(4-Chlorophenyl)acetylimino]-1,5-dideoxy-D-glucitol, tetraacetate; 1,5-[(4-Biphenyl)acetylimino]-1,5-dideoxy-D-glucitol, tetraacetate; 1,5-(Benzyloxycarbonylimino)-1,5-dideoxy-D-glucitol, tetrabutyrate; 1,5-Dideoxy-1,5-imino-D-glucitol, tetrabutyrate; 3,4,5-piperidinetriol, 1-propyl-2-(hydroxymethyl)-, (2S,3R,4R,5S); 3,4,5-piperidinetriol, 1-pentyl-2-(hydroxymethyl)-, (2S,3R,4R,5S); 3,4,5-piperidinetriol, 1-heptyl-2-(hydroxymethyl)-, (2S,3R,4R,5S); 3,4,5-piperidinetriol, 1-butyl-2-(hydroxymethyl)-, (2S,3S,4R,5S); 3,4,5-piperidinetriol, 1-nonyl-2-(hydroxymethyl)-, (2S,3R,4R,5S); 3,4,5-piperidinetriol, 1-(1-ethyl) propyl-2-(hydroxymethyl)-, (2S,3R,4R,5S); 3,4′,5-piperidinetriol, 1-(3-methyl)butyl-2-(hydroxymethyl)-, (2S,3R,4R,5S); 3,4,5-piperidinetriol, 1-(2-phenyl)ethyl-2-(hydroxymethyl)-, (2S,3R,4R,5S); 3,4,5-piperidinetriol, 1-(3-phenyl) propyl-2-(hydroxymethyl)-, (2S,3R,4R,5S); 3,4,5-piperidinetriol, 1-(1-ethyl)hexyl-2-(hydroxymethyl)-, (2S,3R,4R,5S); 3,4,5-piperidinetriol, 1-(2-ethyl)butyl-2-(hydroxymethyl)-, (2S,3R,4R,5S); 3,4,5-piperidinetniol, 1-[(2R)-(2-methyl-2-phenyl)ethyl]-2-(hydroxymethyl)-, (2S,3R,4R,5S); 3,4,5-piperidinetriol, 1-[(2S)-(2-methyl-2-phenyl)ethyl]-2-(hydroxymethyl)-, (2S,3R,4R,5S), β-L-homofuconojirimycin; propyl 2-acetamido-2-deoxy-4-O-(#-D-galactopyranosyl)-3-O-(2-(N-(β-L-homofaconojirimycinyl))ethyl)-α-D-glucopyranoside; ido-N-(5-adamantane-1-yl-methoxy-pentyl)deoxynojirimycin; N-(adamantane-1-yl-methoxypentyl)-L-ido-deoxynojirimycin; N-(adamantane-1-yl-methoxypentyl)-D-galacto-deoxynojirimycin; C1-beta-(adamantane-1-yl-methoxypentyl)-deoxynojirimycin; N-methyl-C1-beta-(adamantane-1-yl-methoxypentyl)-deoxynojirimycin; N-butyl-C1-beta-(adamantane-1-yl-methoxypentyl)-deoxynojirimycin; 2-O-(adamantane-1-yl-methoxypentyl)-deoxynojirimycin; N-methyl-2-O-(adamantane-1-yl-methoxy-pentyl)-deoxynojirimycin; N-butyl-2-O-(adamantane-1-yl-methoxy-pentyl)-deoxynojirimycin; N-benzyloxycarbonyl-2-O-(adamantane-1-yl-methoxypentyl)-3,4,6-tri-O-benzyl-deoxy-nojirimycin; and N-(5-adamantane-1-yl-methoxy-pentyl)deoxynojirimycin.

Methods of synthesizing such iminosugar compounds are known and are described, for example, in WO 02/055498 and in U.S. Pat. Nos. 5,622,972, 4,246,345, 4,266,025, 4,405,714, and 4,806,650, U.S. patent application Ser. No. 07/851,818, filed Mar. 16, 1992, US2007/0066581 and EP1528056. For example, N-nonyl-DNJ and N-decyl-DNJ can be conveniently prepared by the N-nonylation or N-decylation, respectively, of 1,5-dideoxy-1,5-imino-D-glucitol (DNJ) by methods analogous to the N-butylation of DNJ as described in Example 2 of U.S. Pat. No. 4,639,436 by substituting an equivalent amount of n-nonylaldehyde or n-decylaldehyde for n-butylraldehyde. The starting materials are readily available from many commercial sources.

Typically, the compound of formula (I) employed is N-butyldeoxynojirimycin (NB-DNJ) or N-butyldeoxygalactonojirimycin (NB-DGJ). More typically, the compound of formula (I) is NB-DNJ.

NB-DGJ is the galactose analogue of NB-DNJ. NB-DGJ inhibits GSL biosynthesis comparably to NB-DNJ but lacks certain side effect activities associated with NB-DNJ. There has been extensive use of NB-DGJ in mouse models of GSL storage diseases and it is very well tolerated. Thus, in one embodiment, the compound of formula (I) employed is NB-DGJ.

In one embodiment the invention provides the use, in the manufacture of a medicament for the treatment of a glycolipid-mediated autoimmune disease, of a compound of the following formula (II):

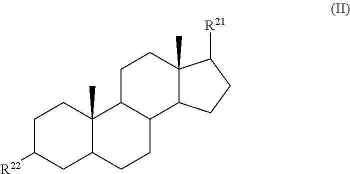

wherein:

R²¹ is selected from oxo, -L³⁰-R²³, -L³⁰-C(O)N(H)—R²⁴ and a group of the following formula (VI):

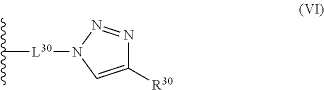

L³⁰ is substituted or unsubstituted C₁₋₂₀ alkylene which is optionally interrupted by N(R′), O, S or arylene;

R²³ is carboxyl, hydroxyl, ester, phosphonate ester, phosphate ester, phosphoric acid and phosphonic acid;

R²⁴ is C₁₋₂₀ alkyl which is unsubstituted or substituted with one or more groups selected from carboxyl, hydroxyl, ester, phosphonate ester, phosphate ester, phosphoric acid and phosphonic acid, wherein said C₁₋₂₀ alkyl is optionally interrupted by N(R′), O, S or arylene;

R³⁰ is C₁₋₂₀ alkyl which is unsubstituted or substituted with one or more groups selected from carboxyl, hydroxyl, ester, amino, phosphonate ester, phosphate ester, phosphoric acid and phosphonic acid, wherein said C₁₋₂₀ alkyl is optionally interrupted by N(R′), O, S or arylene; and

R²² is hydroxyl, oxo, acyloxy, phosphoric acid or —OC(O)-alk-C(O)OH, wherein alk is substituted or unsubstituted C₁₋₂₀ alkylene which is optionally interrupted by N(R′), O, S or arylene,

or a pharmaceutically acceptable salt thereof.

Typically, in the compounds of formula (II), R²¹ is selected from oxo, -L³⁰-R²³, -L³⁰-C(O)N(H)—R²⁴ and a group of the following formula (VI):

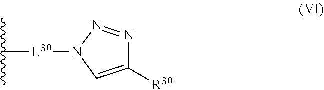

wherein

L³⁰ is substituted or unsubstituted C₁₋₆ alkylene,

R¹³ is hydroxyl, carboxyl, ester or phosphate ester, R²⁴ is C₁₋₆ alkyl which is unsubstituted or substituted with one or two carboxyl groups, and

R³⁰ is C₁₋₆ alkyl which is unsubstituted or substituted with one or two groups selected from hydroxyl, carboxyl, amino and phosphonate ester.

More typically, R²¹ is a group selected from oxo and the groups having the following structures:

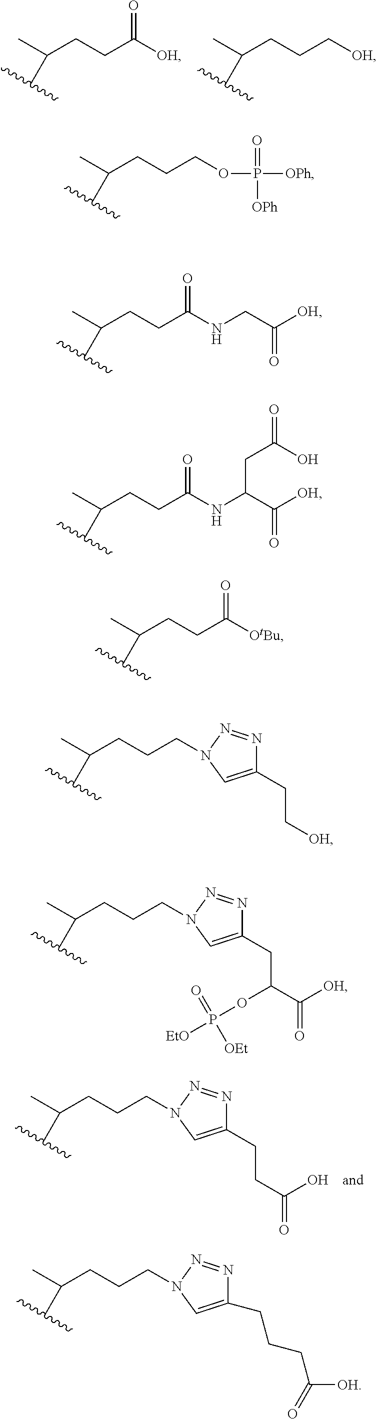

Typically, in the compounds of formula (II), R²² is selected from hydroxyl, oxo, phosphoric acid, —OC(O)—CH₂—CH₂—C(O)OH and —OC(O)—CH(NH₂)—CH₂—C(O)OH.

Table 1 shows examples of compounds of formula (II) which may be employed in the present invention:

TABLE 1

| Compound | R²¹ | R²² |
|---|---|---|
| 1 | ═O | —OC(O)CH₂CH₂C(O)OH |
| 2 | 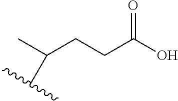 | —OH |
| 3 | ═O | —OC(O)CH(NH₂)CH₂C(O)OH |
| 4 | ═O | —O—P(═O)(OH)₂ |
| 5 | ═O | —OC(O)CH₂CH₂C(O)OH |
| 6 | 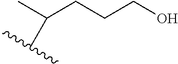 | —OH |
| 7 | 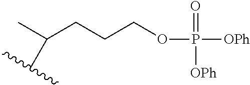 | —OH |
| 8 | 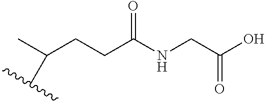 | —OH |
| 9 | 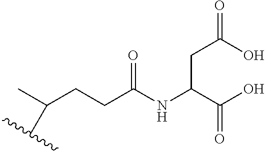 | —OH |
| 10 | 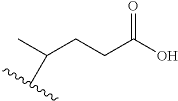 | ═O |
| 11 | 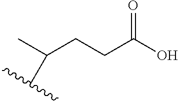 | —OC(O)CH₂CH₂C(O)OH |
| 12 | 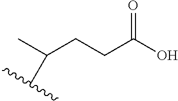 | —OC(O)CH(NH₂)CH₂C(O)OH |
| 13 | 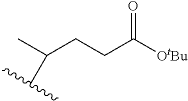 | —OC(O)CH₂CH₂C(O)OH |
| 14 | 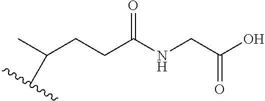 | —OC(O)CH₂CH₂C(O)OH |
| 15 | 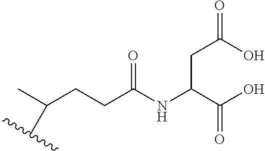 | —OC(O)CH₂CH₂C(O)OH |
| 16 | 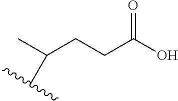 | —OC(O)CH(NH₂)CH₂C(O)OH |
| 17 | 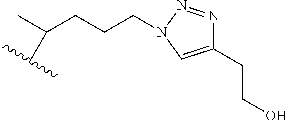 | —OH |
| 18 | 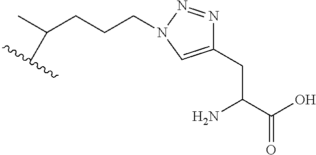 | —OH |
| 19 | 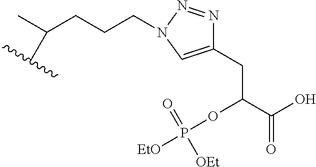 | —OH |
| 20 | 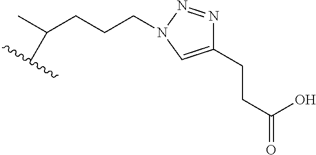 | —OH |
| 21 | 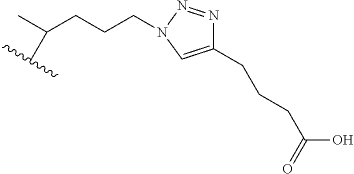 | —OH |

The synthesis of such compounds is described in Chang K H et al. Chem Commun (Camb). 2006 Feb. 14; (6):629-31.

In one embodiment the invention provides the use, in the manufacture of a medicament for the treatment of a glycolipid-mediated autoimmune disease, of a compound of the following formula (III):

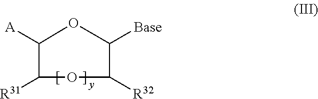

wherein:

Base is selected from a group of any one of the following formulae (a), (b), (c), (d), (e), (f) and (g):

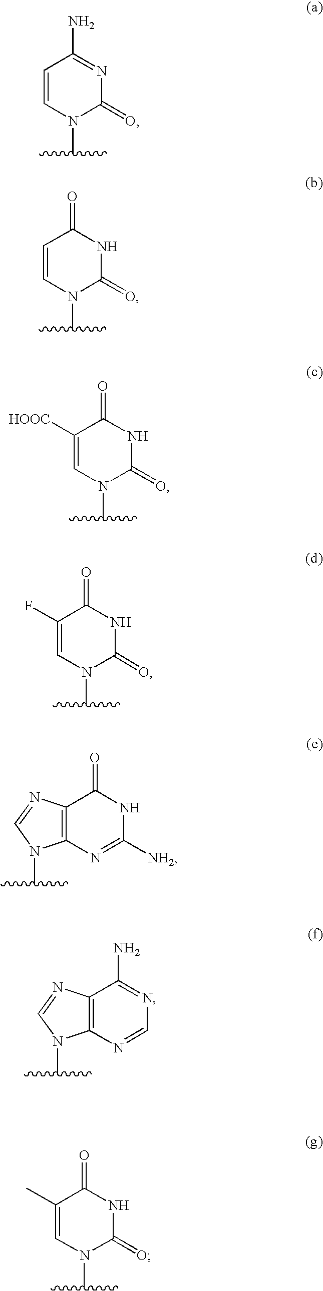

y is 0 or 1;

R³¹ is OH; R³² is H or OH; or, provided that y is 0, R³¹ and R³² together form —O—C(R³³)(R³⁴)—O—, wherein R³³ and R³⁴ are independently selected from H and methyl;

A is substituted or unsubstituted C₁₋₂₀ alkyl, substituted or unsubstituted C₁₋₂₀ alkylene-aryl, substituted or unsubstituted C₁₋₂₀ alkylene-C₃₋₂₀ heteroaryl, substituted or unsubstituted C₁₋₂₀ alkylene-C₃₋₂₅ cycloalkyl or substituted or unsubstituted C₁₋₂₀ alkylene-C₃₋₂₀ heterocyclyl, wherein said C₁₋₂₀ alkyl and C₁₋₂₀ alkylene are optionally interrupted by N(R′), O, S or arylene, wherein R′ is H, C₁₋₆ alkyl or aryl, or A is a group of any one of the following formulae (g) to (k):

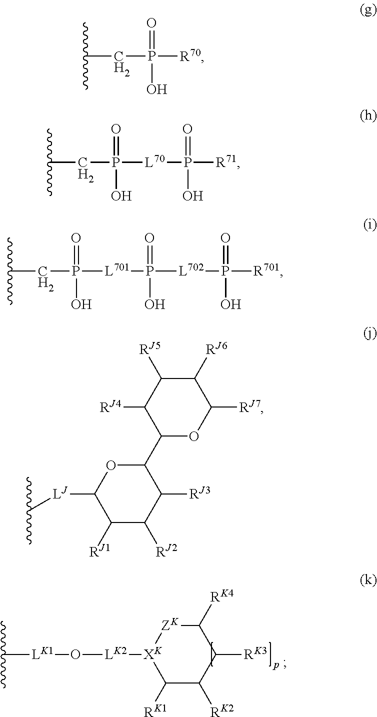

L⁷⁰, L⁷⁰¹ and L⁷⁰² are independently selected from —O—, —C(R³⁵)(R³⁶)— and —NH—, wherein R³⁵ and R³⁶ are independently selected from H, OH and CH₃;

R⁷⁰, R⁷¹ and R⁷⁰¹ are selected from OH, substituted or unsubstituted C₁₋₂₀ alkyl, substituted or unsubstituted C₁₋₂₀ alkoxy, substituted or unsubstituted C₁₋₁₀ alkylamino and -L⁷¹-(X²)ₘ-L⁷²-R⁷²; wherein m is 0 or 1; X² is O, S, —C(R⁴⁵)(R⁴⁶)— or —O—C(R⁴⁵)(R⁴⁶)—, wherein R⁴⁵ and R⁴⁶ are independently selected from H, OH, phosphonic acid or a phosphonic acid salt; L⁷¹ and L⁷² are independently selected from a single bond and substituted or unsubstituted C₁₋₂₀ alkylene, which C₁₋₂₀ alkylene is optionally interrupted by N(R′), O, S or arylene, wherein R′ is H, C₁₋₆ alkyl or aryl; and R⁷² is C₃₋₂₅ cycloalkyl or C₃₋₂₀ heterocyclyl;

L^{J} is substituted or unsubstituted C₁₋₂₀ alkylene;

R^{J1}, R^{J2}, R^{J3}, R^{J4}, R^{J5}, R^{J6} and R^{J7}, which are the same or different, are independently selected from hydrogen, hydroxyl, carboxyl, amino, thiol, substituted or unsubstituted C₁₋₂₀ alkyl, substituted or unsubstituted C₁₋₂₀ alkoxy, substituted or unsubstituted aryloxy, acyl, ester, acyloxy, C₁₋₁₀ alkylamino, di(C₁₋₁₀)alkylamino, amido, acylamido, —N(H)C(O)CH═CH—R¹³, —O—C₃₋₂₅ cycloalkyl and —O—C₃₋₂₀ heterocyclyl, wherein said C₁₋₂₀ alkyl is optionally interrupted by N(R′), O, S or arylene, and wherein R^{J8} is substituted or unsubstituted C₁₋₂₀ alkyl;

L^{K1} and L^{K2}, which are the same or different, are independently selected from a single bond and substituted or unsubstituted C₁₋₂₀ alkylene;

X^{K} is N or (R^{K6}), wherein R^{K6} is H, COOH or ester;

Z^{K} is O or CH(R^{K5});

p is 0 or 1; and

R^{K1}, R^{K2}, R^{K3}, R^{K4} and R^{K5}, which are the same or different, are independently selected from hydrogen, hydroxyl, carboxyl, amino, thiol, substituted or unsubstituted C₁₋₂₀ alkyl, substituted or unsubstituted C₁₋₂₀ alkoxy, substituted or unsubstituted aryloxy, acyl, ester, acyloxy, C₁₋₁₀ alkylamino, di(C₁₋₁₀)alkylamino, amido, acylamido, —O—C₃₋₂₅ cycloalkyl and —O—C₃₋₂₀ heterocyclyl, wherein said C₁₋₂₀ alkyl is optionally interrupted by N(R′), O, S or arylene;

or a pharmaceutically acceptable salt thereof.

Typically, in the compounds of formula (III), Base is selected from (a) (i.e. cytosine), (b) (i.e. uracil), (c) (i.e. carboxy-substituted uracil), (d) (i.e. fluoro-substituted uracil) and (e) (i.e. guanine).

Typically, y is 0 and R³¹ and R³² are both —OH.

In one embodiment, y is 1, and R³¹ and R³² together form —O—C(R³³)(R³⁴)—O—, wherein R³³ and R³⁴ are as defined above. Typically, in that embodiment, Base is cytosine. Typically, R³³ and R³⁴ are both methyl.

Typically, A is either (g), (h) or (i). More typically, A is either (g) or (h). Typically, L⁷⁰, L⁷⁰¹ and L⁷⁰² are independently selected from O, CH₂, CHOH, C(OH)(CH₃) and NH. More typically, L⁷⁰, L⁷⁰¹ and L⁷⁰² are independently selected from O or CH₂. Even more typically, L⁷⁰, L⁷⁰¹ and L⁷⁰² are O.

Typically, R⁷⁰, R⁷¹ and R⁷⁰¹ are -L⁷¹-(X²)ₘ-L⁷²-R⁷²; wherein m, X², L, L² and R⁷² are as defined above. Typically, L⁷¹ is a single bond or a substituted or unsubstituted C₁₋₆ alkyl group. Typically, L⁷² is a single bond or a substituted or unsubstituted C₁₋₆ alkyl group. Typically, m is 1 and X² is O, S, —CH(OH)— or —O—CH(R⁴⁶)— wherein R⁴⁶ is phosphonic acid or a phosphonic acid salt. Alternatively, m may be 0.

Typically, R⁷² is a group of the following formula (a′):

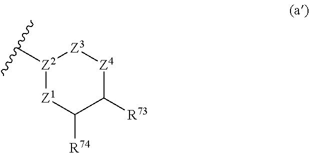

wherein

Z¹ is CHR^{Z1} and Z² is CR^{Z2} wherein Z¹ and Z² are connected by a single bond, or Z¹ is CH and Z² is C wherein Z¹ and Z² are connected by a double bond;

Z³ is CHR^{Z3} or O, and Z⁴ is CHR^{Z4} or O, provided that Z³ and Z⁴ are not both O;

R^{Z2} is H or COOR^{Z2a}, wherein R^{Z2a} is H, methyl or ethyl;

R^{Z1}, R^{Z3}, R^{Z4}, R⁷³ and R⁷⁴, which may be the same or different, are independently selected from hydrogen, hydroxyl, carboxyl, amino, thiol, substituted or unsubstituted C₁₋₂₀ alkyl, substituted or unsubstituted C₁₋₂₀ alkoxy, substituted or unsubstituted aryloxy, acyl, ester, acyloxy, C₁₋₁₀ alkylamino, di(C₁₋₁₀)alkylamino, amido, acylamido —O—C₃₋₂₅ cycloalkyl and —O—C₃₋₂₀ heterocyclyl, wherein said C₁₋₂₀ alkyl is optionally interrupted by N(R′), O, S or arylene.

Typically, R^{Z1} is H, OH, NHC(O)CH₃ or phenoxy. Typically, R^{Z2} is H or COOH. Typically, R^{Z3} is H, OH, NHC(O)CH₃ or phenoxy. Typically R^{Z4}, R⁷³ and R⁷⁴ are independently selected from H; OH; CH₂OH; NH₂; NH₃⁺; NHC(O)CH₃; phenoxy; C₁₋₄ alkyl substituted with 1 to 3 hydroxyl, unsubstituted C₁₋₄alkoxy or acyloxy groups; and C₁₋₄ alkoxy substituted with 1 to 3 hydroxyl, unsubstituted C₁₋₄ alkoxy or acyloxy groups. More typically, R^{Z4}, R⁷³ and R⁷⁴ are independently selected from H; OH; CH₂OH; NH₃⁺; NHC(O)CH₃; phenoxy; methyl; ethyl; propyl and butyl; which methyl, ethyl, propyl or butyl are substituted with one, two, three and four hydroxyl groups respectively. Even more typically, R^{Z4}, R⁷³ and R⁷⁴ are independently selected from H, OH, CH₂OH, NH₃⁺, NHC(O)CH₃, phenoxy, —OCH₂CH₂OH and —CH(OH)CH(OH)CH₂OH.

When R⁷² is (a′), typically L⁷¹ is a single bond, L⁷² is a single bond, m is 1 and X² is O or S. In another embodiment, when R⁷² is (a′), typically L⁷¹ is CH₂, L⁷² is a single bond, and m is 0. In another embodiment, when R⁷² is (a′), typically L⁷² is a single bond, L⁷² is a single bond, and m is 0.

In one embodiment, R⁷² is a group of the following formula (b′):

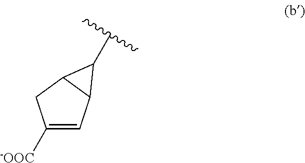

Typically, when R⁷² is (b′), L⁷¹ is a single bond, L⁷² is substituted or unsubstituted C₁₋₄ alkyl, m is 1 and X² is O or S. More typically, when R⁷² is (b′), L⁷¹ is a single bond, L⁷² is CH₂, m is 1 and X² is 0.

In one embodiment, R⁷⁰, R⁷¹ and R⁷⁰¹ are -L⁷¹-(X²)ₘ-L⁷²-R⁷², wherein L⁷¹, X² and m are as defined above and L⁷² and R⁷² together form a group of the following formula (c′):

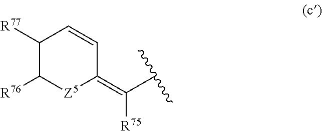

wherein:

R⁷⁵ is H, substituted or unsubstituted C₁₋₄ alkyl, or phosphonic acid;

Z⁵ is O or CH(R^{Z5});

R⁷⁶, R⁷⁷ and R^{Z5}, which may be the same or different, are independently selected from hydrogen, hydroxyl, carboxyl, amino, thiol, substituted or unsubstituted C₁₋₂₀ alkyl, substituted or unsubstituted C₁₋₂₀ alkoxy, substituted or unsubstituted aryloxy, acyl, ester, acyloxy, C₁₋₁₀ alkylamino, di(C₁₋₁₀)alkylamino, amido, acylamido —O—C₃₋₂₅ cycloalkyl and —O—C₃₋₂₀ heterocyclyl, wherein said C₁₋₂₀ alkyl is optionally interrupted by N(R′), O, S or arylene.

Typically in this embodiment, L⁷¹ is a single bond. Typically in this embodiment, m is 1 and X² is O or S. More typically, m is 1 and X² is 0. Typically, R⁷⁵ is H or phosphonic acid. Typically, R⁷⁶, R⁷⁷ and R^{Z5}, which are the same or different, are independently selected from H; OH; CH₂OH; NH₂; NH₃⁺; NHC(O)CH₃; phenoxy; C₁₋₄ alkyl substituted with 1 to 3 hydroxyl, unsubstituted C₁₋₄ alkoxy or acyloxy groups; and C₁₋₄ alkoxy substituted with 1 to 3 hydroxyl, unsubstituted C₁₋₄ alkoxy or acyloxy groups. More typically, R⁷⁶, R⁷⁷ and R^{Z5} are independently selected from H; OH; CH₂OH; NH₃⁺; NHC(O)CH₃; phenoxy; methyl; ethyl; propyl and butyl; which methyl, ethyl, propyl or butyl are substituted with one, two, three and four hydroxyl groups respectively. Even more typically, R⁷⁶, R⁷⁷ and R^{Z5} are independently selected from H, OH, CH₂OH, NHC(O)CH₃, phenoxy, —OCH₂CH₂OH and —CH(OH)CH(OH)CH₂OH. Typically, R^{Z5} is H. Typically R⁷⁶ is selected from H, OH, CH₂OH, NHC(O)CH₃, phenoxy, —OCH₂CH₂OH and —CH(OH)CH(OH)CH₂OH. Typically, R⁷⁷ is H, OH or NHC(O)CH₃. More typically, R⁷⁶ is —CH(OH)CH(OH)CH₂OH or phenoxy and R⁷⁷ is NHC(O)CH₃. Typically, Z⁵ is O.

In one embodiment, R⁷⁰, R⁷¹ and R⁷⁰¹ are selected from a C₁₋₆ alkyl group substituted with phosphonic acid, carboxyl or —CH₃C(═CH₂)COOH; a C₁₋₁₀ alkylamino group substituted with a carboxyl group; and a C₁₋₁₀ alkoxy group substituted with one or more groups selected from phenyl, phosphonic acid, phosphonic acid salt, 2-furyl, carboxyl, —COONa or benzyl. In particular, R⁷⁰, R⁷¹ and R⁷⁰¹ may be selected from a methyl group substituted with phosphonic acid, carboxyl or —CH₃C(═CH₂)COOH; an ethyl group substituted with phosphonic acid or carboxyl; a C₁₋₁₀ alkylamino group substituted with a carboxyl group; and OCH(Z⁶)(Z⁷), wherein Z⁶ and Z⁷, which may be the same or different, are independently selected from phenyl, phosphonic acid, phosphonic acid salt, 2-furyl, carboxyl, —COONa and benzyl.

In one embodiment, A in formula (III) is selected from substituted C₁₋₂₀ alkyl, substituted C₁₋₂₀ alkylene-aryl and substituted C₁₋₂₀ alkylene-C₃₋₂₀ heteroaryl. For instance, in one embodiment, A is a group of the following formula (d′):

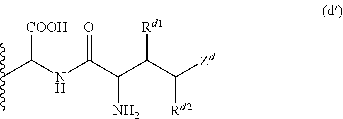

wherein Z^{d} is substituted or unsubstituted phenyl, substituted or unsubstituted pyridyl or —CH₂OC(O)NH₂; R^{d1} is OH, C₁₋₄ alkyl or C₁₋₄ alkoxy; and R^{d2} is OH, C₁₋₄ alkyl or C₁₋₄ alkoxy. In one embodiment, A is a group of one of the following formula (d″) and (d′″):

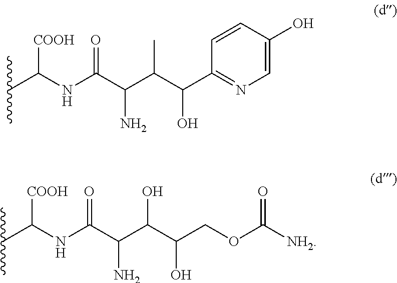

Typically, when A of formula (III) is (j), L^{J} is substituted or unsubstituted C₁₋₄ alkylene. More typically, L^{J} is C₁₋₄ alkylene substituted with a hydroxyl group. For instance, L^{J} may be —CH(OH)CH₂—. Typically, R^{J1} and R^{J2} are selected from H, OH, CH₂OH, NHC(O)CH₃ and unsubstituted C₁₋₄ alkyl. More typically R^{1e} and R³² are both OH. Typically, R^{J3} is —N(H)C(O)CH═CH—R^{J8}. Typically R^{J8} is a C₁₋₂₀ alkyl group substituted with an unsubstituted C₁₋₄ alkyl group, for instance a C₁₁ alkyl group substituted with a methyl group. Typically, R^{J4}, R^{J5}, R^{J6} and R^{J7} are selected from H, OH, CH₂OH, NHC(O)CH₃ and unsubstituted C₁₋₄ alkyl. More typically, R^{J7} is CH₂OH, R^{J6} and R^{J5} are both OH, and R^{J4} is NHC(O)CH₃.

Typically, when A of formula (M) is (k), L^{K1} is unsubstituted C₁₋₄ alkylene. For instance, L^{K1} may be methylene, ethylene or propylene. Typically, L^{K2} is a single bond or C₁₋₆ alkylene, which C₂₋₆ alkylene is unsubstituted or substituted with an oxo group. For instance, in one embodiment, L^{K2} is either a single bond or —C(O)CH₂C(O)—. Typically X^{K} is N, CH, COOH or COOCH₃. Typically Z^{K} is O or CH(CH₂OH).

In one embodiment, X^{K} is N, Z^{K} is CH(R^{K5}) and p is 0. Typically, in this embodiment, R^{K1}, R^{K2}, R^{K4} and R^{K5}, which are the same or different, are independently selected from H, OH, CH₂OH, NH₂, NHC(O)CH₃, phenoxy and C₁₋₄ alkyl, which C₁₋₄ alkyl is unsubstituted or substituted with 1 to 3 hydroxyl groups. More typically, in this embodiment, R^{K5} and R^{K1} are both CH₂OH, and R^{K2} and R^{K4} both OH.

In another embodiment, X^{K} is CH, COOH or ester (for instance COOCH₃), Z^{K} is O and p is 1. Typically, in this embodiment, R^{K1} and R^{K4}, which are the same or different, are independently selected from H, OH, CH₂OH, NH₂, NHC(O)CH₃, acyloxy, phenoxy and C₁₋₄ alkyl, which C₁₋₄ alkyl is unsubstituted or substituted with 1 to 3 hydroxyl or acyloxy groups. More typically, R^{K1} and R^{K4} are independently selected from H, OH, CH₂OH, —CH(OH)CH(OH)CH₂OH and —CH(OAc)CH(OAc)CH₂OAc, wherein Ac is —C(O)CH₃. Typically, in this embodiment, R^{K2} and R^{K3}, which are the same or different, are independently selected from H; OH; CH₂OH; NH₂; NHC(O)CH₃; acyloxy; phenoxy; C₁₋₄ alkyl, which C₁₋₄ alkyl is unsubstituted or substituted with 1 to 3 hydroxyl or acyloxy groups; and a group of the following formula (e′):

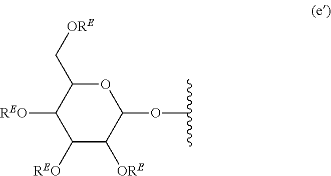

wherein each R^{E} is either H or acyl. Typically each R^{E} is H.

In one embodiment, the inhibitor of glycolipid biosynthesis is of formula (III) wherein Base is selected from (a), (b), (c), (d) and (e); y is 0 and R³¹ and R³² are both —OH; A is either (g), (h) or (i); L⁷⁰, L⁷⁰¹ and L⁷⁰² are selected from O, CH₂, CHOH, C(OH)(CH₃) and NH; and R⁷⁰, R⁷¹ and R⁷⁰¹ are as defined above.

Table 2 shows examples of compounds of formula (III) which may be employed in the present invention. The compounds in Table 2 are described in R. Wang et al., Bioorg. & Med. Chem., Vol. 5, No. 4, pp 661-672, 1997; X. Wang et al., Medicinal Research Reviews, Vol. 23, No. 1, 32-47, 2003; Schafer et al., J. Org. Chem. 2000, 65, 24-29; and Qiao et al., J. Am. Chem. Soc., 1996, 118, 7653-7662.

TABLE 2

| Compound | Compound structure |
|---|---|
| 22 | 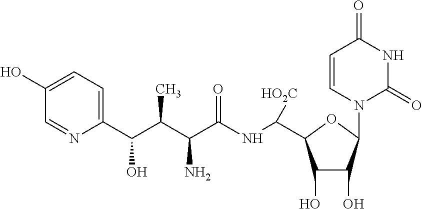 |
| 23 | 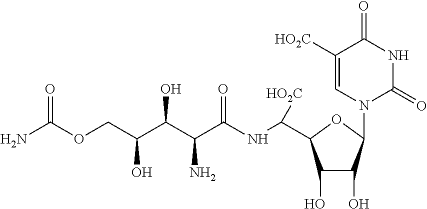 |
| 24 | 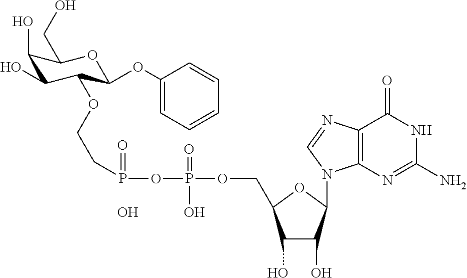 |
| 25 | 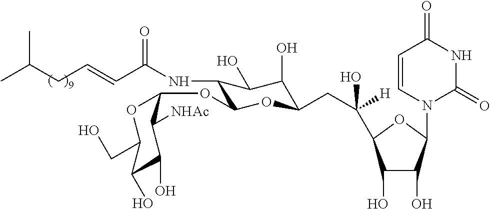 |
| 26 | 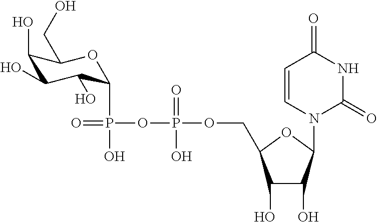 |
| 27 | 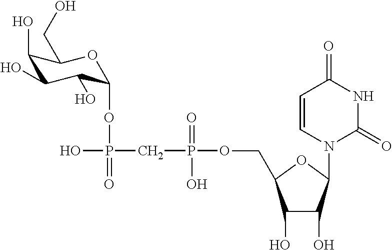 |
| 28 | 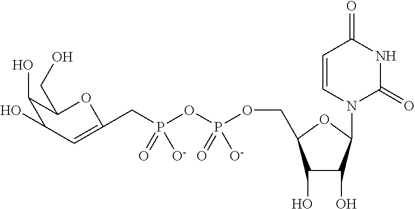 |
| 29 | 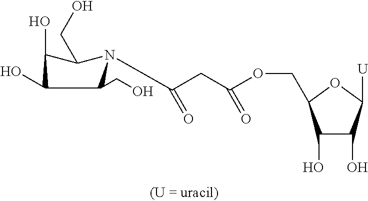 |
| 30 | 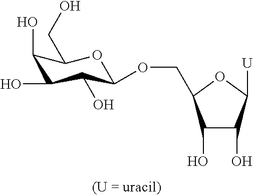 |
| 31 | 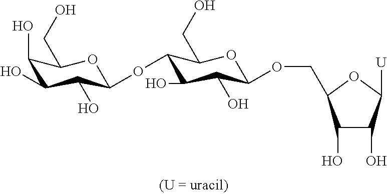 |
| 32 | 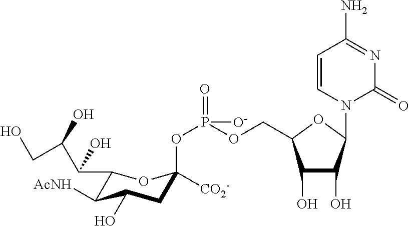 |
| 33 | 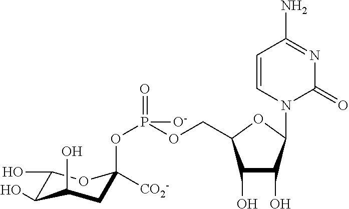 |
| 34 | 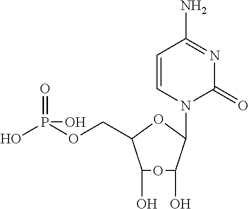 |
| 35 | 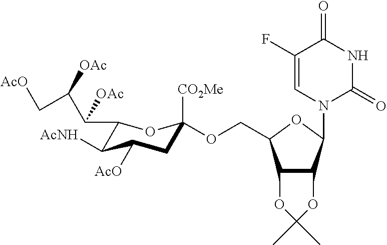 |
| 36 | 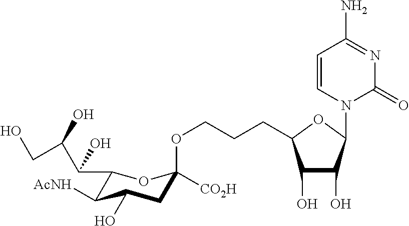 |
| 37 | 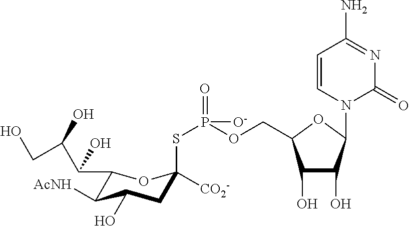 |
| 38 (a and b) | 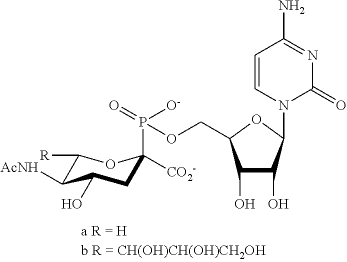 |
| 40 | 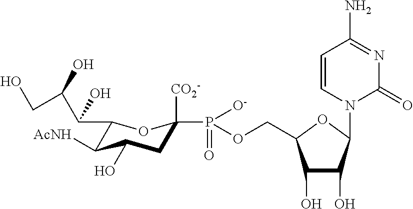 |
| 41 (a to e) | 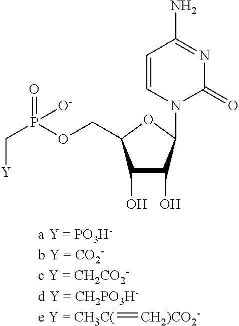 |
| 42 | 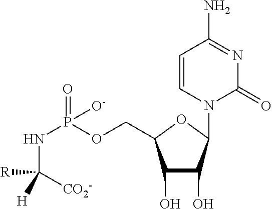 |
| 43 | 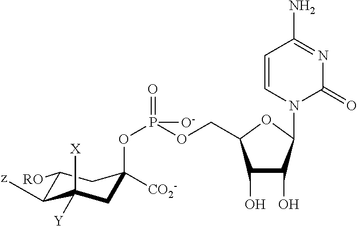 |
| 44 (a and b) | 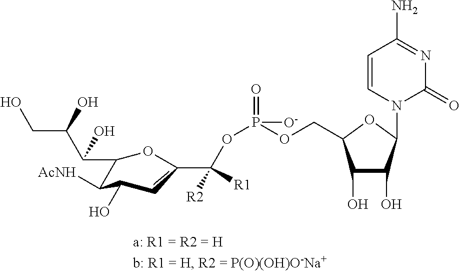 |
| 45 | 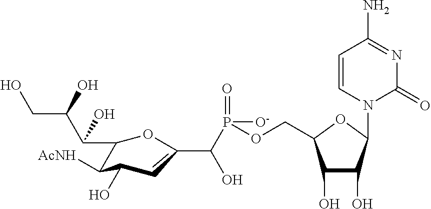 |
| 46 (a to h) | 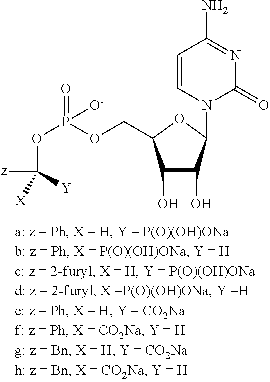 |
| 47 (a and b) | 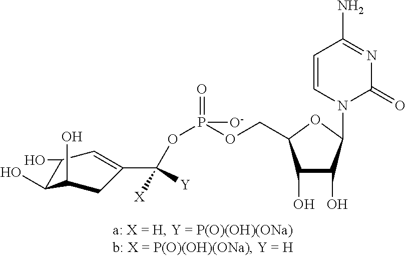 |
| 48 | 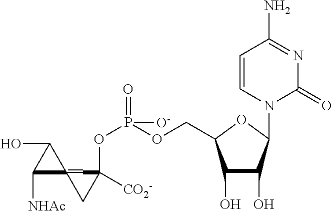 |
| 49 | 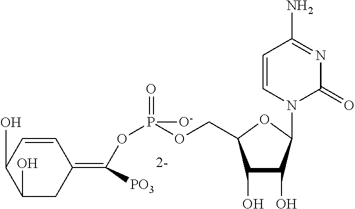 |
| 50 | 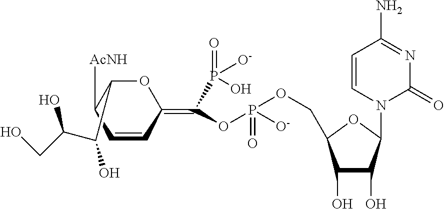 |
| 51 | 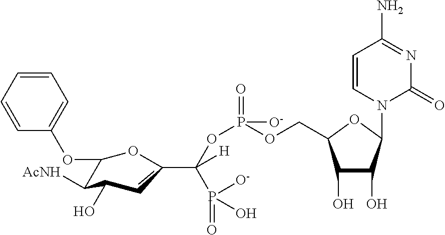 |
| 52 | 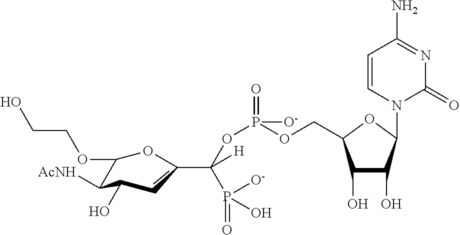 |
| 53 | 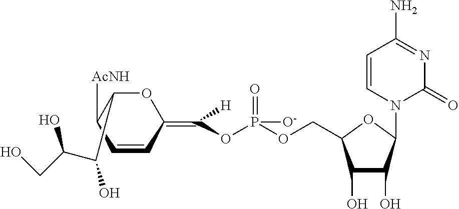 |
| 54 | 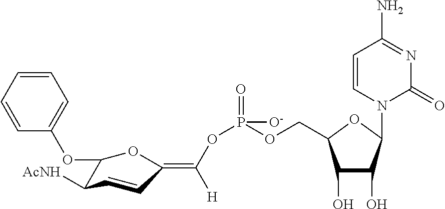 |
| 55 | 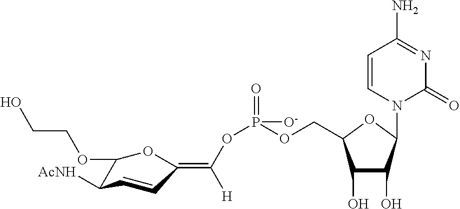 |
| 56 | 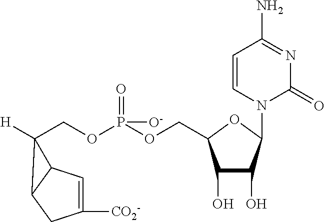 |
| 57 | 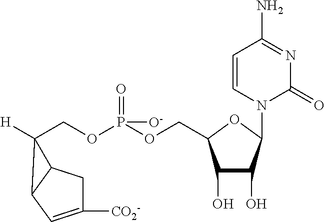 |
| 58 (a to c) | 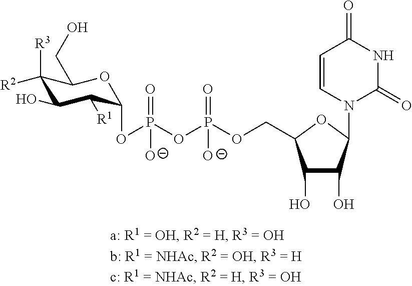 |
| 59 (a to c) | 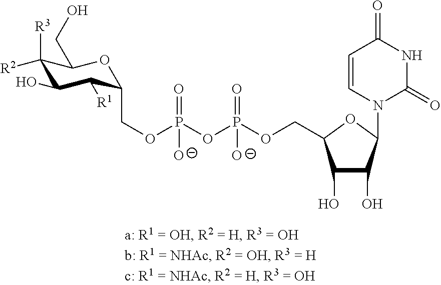 |
| 60 | 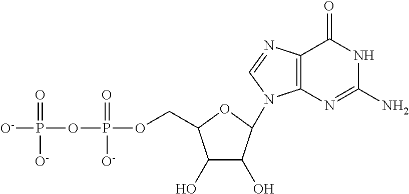 |

In one embodiment the invention provides the use, in the manufacture of a medicament for the treatment of a glycolipid-mediated autoimmune disease, of a compound of the following formula (IV):

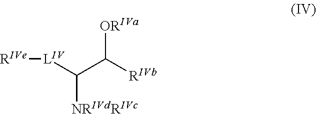

in which:

R^{IVa} and R^{IVd}, which are the same or different, are independently selected from H, substituted or unsubstituted C₁₋₆ alkyl or substituted or unsubstituted phenyl;

R^{IVb} is H, substituted or unsubstituted aryl, —CH═CHR^{IVf}, or substituted or unsubstituted C₁₋₂₀ alkyl, which C₁₋₂₀ alkyl is optionally interrupted by N(R′), O, S or arylene wherein R′ is H, C₁₋₆ alkyl or aryl;

R^{IVc} is H, substituted or unsubstituted C₁₋₆ alkyl, substituted or unsubstituted phenyl or —C(O)R^{IVg};

R^{IVf} is H or substituted or unsubstituted C₁₋₂₀ alkyl, which C₁₋₂₀ alkyl is optionally interrupted by N(R′), O, S or arylene;

R^{IVg} is H or substituted or unsubstituted C₁₋₂₀ alkyl, which C₁₋₂₀ alkyl is optionally interrupted by N(R′), O, S or arylene;

R^{IVe} is H, hydroxyl, carboxyl, amino, thiol, substituted or unsubstituted C₁₋₂₀ alkyl, substituted or unsubstituted C₁₋₂₀ alkoxy, substituted or unsubstituted aryloxy, acyl, ester, acyloxy, C₁₋₁₀ alkylamino, di(C₁₋₁₀)alkylamino, amido, acylamido, —O—C₃₋₂₅ cycloalkyl, —O—C₃₋₂₀ heterocyclyl, substituted or unsubstituted aryl, substituted or unsubstituted C₃₋₂₀ heteroaryl, substituted or unsubstituted C₃₋₂₅ cycloalkyl or substituted or unsubstituted C₃₋₂₀ heterocyclyl, which C₁₋₂₀ alkyl is optionally interrupted by N(R′), O, S or arylene; and

L^{IV} is substituted or unsubstituted C₁₋₂₀ alkylene which C₁₋₂₀ alkylene is optionally interrupted by N(R′), O, S or arylene;

or a pharmaceutically acceptable salt thereof.

Typically, R^{IVa} is H. Typically, R^{IVd} is H.

Typically, R^{IVc} is H or —C(O)R^{IVg}. More typically, R^{IVc} is —C(O)R^{IVg}. Typically, R^{IVg} is unsubstituted C₁₋₂₀ alkyl. R^{IVg} may be, for instance, C₅H₁₁, C₉H₁₉ or C₁₅H₃₁.

Typically L^{IV} is CH₂.

Typically, R^{IVb} is —CH═CHR^{IVf}. Typically, R^{IVf} is unsubstituted C₁₋₂₀ alkyl. Thus, R^{IVf} may be, for instance, —C₁₃H₂₇.

Alternatively, R^{IVb} may be a group of the following formula (IVa):

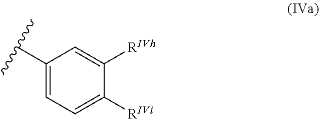

in which R^{IVh} is H, C₁₋₆ alkyl, phenyl or, together with R^{IVi} a bidentate group of the structure —O-alk-O—; and R^{IVi} is H, C₁₋₆ alkyl, phenyl or, together with R^{IVh} a bidentate group of the structure —O-alk-O—, wherein alk is substituted or unsubstituted C₁₋₆ alkylene.

Typically, R^{IVh} is H or, together with R^{IVi} a bidentate group of the structure —O—CH₂—CH₂—O—. Typically, R^{IVi} is H, OH or, together with R^{IVh} a bidentate group of the structure —O—CH₂—CH₂—O—. More typically, R^{IVh} is H and R^{IVi} is either H or OH. Alternatively, R^{IVi} and R^{IVh} together form a bidentate group of the structure —O-alk-O—.

Typically, R^{IVe} is substituted or unsubstituted aryl, substituted or unsubstituted C₃₋₂₀ heteroaryl, substituted or unsubstituted C₃₋₂₅ cycloalkyl or substituted or unsubstituted C₃₋₂₀ heterocyclyl. More typically, R^{IVe} is substituted or unsubstituted C₃₋₂₀ heterocyclyl, even more typically a substituted or unsubstituted C₄₋₆ heterocyclyl. R^{IVe} may be N-pyrrolidyl or N-morpholinyl, for instance.

Alternatively, R^{IVe} is OH. Typically, when R^{IVe} is OH, L^{IV} is CH₂.

Examples of compounds of formula (IV) include D,L-threo-1-phenyl-2-decanoylamino-3-morpholino-1-propanol (DMP); D,L-threo-1-phenyl-2-hexadecanoylamino-3-morpholino-1-propanol (PPMF); D-threo-1-phenyl-2-palmitoilamino-3-pyrrolidino-1-propanol (P4); 4′-hydroxy-D-threo-1-phenyl-2-palmitoilamino-3-pyrrolidino-1-propanol (4′-hydroxy-P4) and 3′,4′-ethylenedioxy-P4 (EtDO-P4). Such compounds are glycosyltransferase inhibitors and derivatives of sphingosine (“lipid mimics”).

In one embodiment the invention provides the use, in the manufacture of a medicament for the treatment of a glycolipid-mediated autoimmune disease, of a compound of the following formula (V):

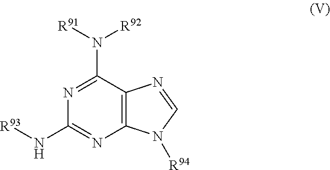

wherein:

R⁹¹ and R⁹², which are the same or different, are independently selected from H, substituted or unsubstituted C₁₋₂₀ alkyl, substituted or unsubstituted aryl and -L⁹¹-R⁹⁵, wherein L⁹¹ is substituted or unsubstituted C₁₋₂₀ alkylene, wherein said C₁₋₂₀ alkyl and said C₁₋₂₀ alkylene are optionally interrupted by N(R′), O, S or arylene wherein R′ is H, C₁₋₆ alkyl or aryl, and wherein R⁹⁵ is substituted or unsubstituted aryl, amino, C₁₋₁₀ alkylamino or di(C₁₋₁₀)alkylamino;

R⁹³ is -L⁹²-R⁹⁶, wherein L⁹² is a single bond or substituted or unsubstituted C₁₋₂₀ alkylene, which C₁₋₂₀ alkylene is optionally interrupted by N(R′), O, S or arylene, and wherein R⁹⁶ is amido or substituted or unsubstituted aryl; and

R⁹⁴ is H or substituted or unsubstituted C₁₋₂₀ alkyl, which C₁₋₂₀ alkyl is optionally interrupted by N(R′), O, S or arylene;

or a pharmaceutically acceptable salt thereof.

Typically, R⁹¹ is H or -L⁹¹-R⁹⁵, wherein L⁹¹ is unsubstituted C₁₋₁₀ alkylene and R⁹⁵ is amino, C₁₋₁₀ alkylamino or di(C₁₋₁₀)alkylamino. More typically, R⁹¹ is H or -L⁹¹-R⁹⁵, wherein L⁹¹ is unsubstituted C₁₋₄ alkylene and R⁹⁵ is amino, C₁₋₁₀ alkylamino or di(C₁₋₁₀)alkylamino. Typically, R⁹² is -L⁹¹-R⁹⁵, wherein L⁹¹ is unsubstituted C₁₋₁₀ alkylene and R⁹⁵ is substituted or unsubstituted aryl. More typically, R⁹² is -L⁹¹-R⁹⁵, wherein L⁹¹ is unsubstituted C₁₋₄ alkylene and R⁹⁵ is substituted or unsubstituted phenyl. Thus, typically, R⁹¹ is H and R⁹² is —C₁₋₄ alkylene-phenyl, wherein said phenyl is substituted or unsubstituted. Typically said phenyl is unsubstituted or mono-substituted with a halo group, for instance with a chloro or fluoro group. Alternatively, R⁹¹ is -L⁹¹-R⁹⁵, wherein L⁹¹ is unsubstituted C₁₋₄ alkylene and R⁹⁵ is amino, C₁₋₁₀ alkylamino or di(C₁₋₁₀)alkylamino and R⁹² is —C₁₋₄ alkylene-phenyl, wherein said phenyl is substituted or unsubstituted. Typically said phenyl is unsubstituted or mono-substituted with a halo group, for instance with a chloro or fluoro group.

Typically, R⁹³ is -L⁹²-R⁹⁶, wherein L⁹² is unsubstituted C₁₋₁₀ alkylene and R⁹⁶ is amido or substituted or unsubstituted aryl. More typically, L⁹² is methylene or ethylene. More typically, R⁹⁶ is amido or substituted or unsubstituted phenyl. Even more typically, R⁹⁶ is —C(O)NH₂ or substituted or unsubstituted phenyl. Typically said phenyl is mono-substituted with a halo group, for instance with a bromo group. Alternatively, said phenyl is unsubstituted.

Typically, R⁹⁴ is C₁₋₁₀ alkyl, which C₁₋₁₀ alkyl is unsubstituted or substituted with a hydroxyl group. More typically R⁹⁴ is selected from methyl, ethyl, propyl, butyl, CH₂OH, hydroxy-substituted ethyl, hydroxy-substituted propyl and hydroxy-substituted butyl. Even more typically, R⁹⁴ is methyl or —CH₂CH₂OH.

Thus, in one embodiment, R⁹¹ is H, —C₁₋₄ alkylene-amino, —C₁₋₄ alkylene-C₁₋₁₀ alkylamino or —C₁₋₄ alkylene-di(C₁₋₁₀)alkylamino;

R⁹² is —C₁₋₄ alkylene-phenyl, wherein said phenyl is substituted or unsubstituted;

R⁹³ is -L 2-R⁹¹, wherein L⁹² is unsubstituted C₁₋₁₀ alkylene and R⁹⁶ is amido or substituted or unsubstituted phenyl; and

R⁹⁴ is C₁₋₁₀ alkyl, which C₁₋₁₀ alkyl is unsubstituted or substituted with a hydroxyl group.

Table 3 shows examples of compounds of formula (V) which may be employed in the present invention. The compounds in Table 3 are described in Armstrong, J. I. et al., Angew. Chem. Int. Ed. 2000, 39, No. 7, p. 1303-1306.

TABLE 3

| Compound | Compound structure |
|---|---|
| 61 | 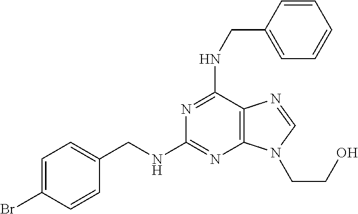 |
| 62 | 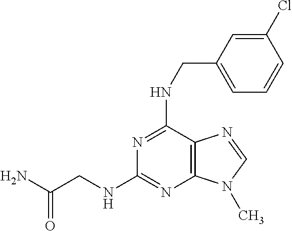 |
| 63 | 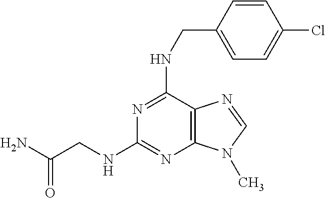 |
| 64 | 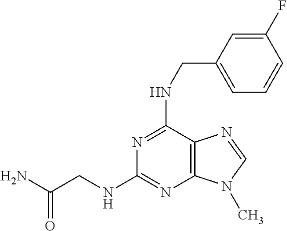 |
| 65 | 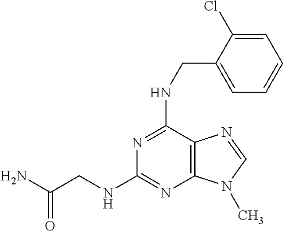 |
| 66 | 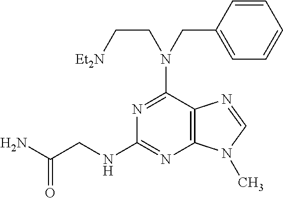 |

In one embodiment the invention provides the use, in the manufacture of a medicament for the treatment of a glycolipid-mediated autoimmune disease, of a compound of the following formula (IX):

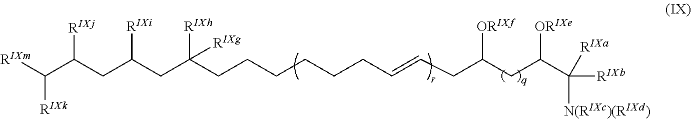

in which:

q is 0 or 1;

r is 0 or 1;

R^{IXa} is H, COOH or an unsubstituted or substituted ester;

R^{IXb} is an unsubstituted or substituted C₁₋₆ alkyl;

R^{IXc} and R^{IXd}, which are the same or different, are each independently selected from H, unsubstituted or substituted C₁₋₆ alkyl and unsubstituted or substituted phenyl;

R^{IXe} and R^{IXf}, which are the same or different, are each independently selected from H, unsubstituted or substituted C₁₋₆ alkyl, unsubstituted or substituted phenyl and unsubstituted or substituted acyl;

either (a) one of R^{IXg} and R^{IX} is H and the other is OR^{IXr}, wherein R^{IXr} is selected from H, unsubstituted or substituted C₁₋₆ alkyl, unsubstituted or substituted phenyl and unsubstituted or substituted acyl, or (b) R^{IXg} and R^{IXh} together form an oxo group;

R^{IXi} is H, unsubstituted or substituted C₁₋₆ alkyl, unsubstituted or substituted C₁₋₆ alkoxy and unsubstituted or substituted phenyl;

R^{IXj} is H, unsubstituted or substituted C₁₋₆ alkyl or a group of the following formula (X):

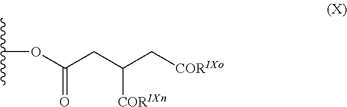

in which R^{IXn} and R^{IXo}, which are the same or different, are each independently selected from OH, unsubstituted or substituted C₁₋₆ alkoxy, unsubstituted or substituted phenoxy, amino, unsubstituted or substituted C₁₋₆ alkylamino and unsubstituted or substituted di(C₁₋₆)alkylamino;

R^{IXk} is H, unsubstituted or substituted C₁₋₆ alkyl or a group of the following formula (XI):

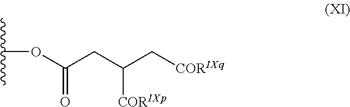

in which R^{IXp} and R^{IXq}, which are the same or different, are each independently selected from OH, unsubstituted or substituted C₁₋₆ alkoxy, unsubstituted or substituted phenoxy, amino, unsubstituted or substituted C₁₋₆ alkylamino and unsubstituted or substituted di(C₁₋₆)alkylamino; and

R^{IXm} is selected from H and unsubstituted or substituted C₁₋₂₀ alkyl, which C₁₋₂₀ alkyl is optionally interrupted by N(R′), O, S or phenylene, wherein R′ is H, C₁₋₆ alkyl or phenyl;

or a pharmaceutically acceptable salt thereof.

In one embodiment, r is 0 and q is 1. Typically, in that embodiment, R^{IXb} is unsubstituted C₁₋₆ alkyl. More typically, R^{IXb} is methyl. Furthermore, R^{IXa} is typically H. Usually R^{IXc} and R^{IXd} are independently selected from H and unsubstituted C₁₋₆ alkyl. More typically, however, R^{IXc} and R^{IXd} are both H. Typically, R^{IXe} and R^{IXf} are independently selected from H and unsubstituted C₁₋₆ alkyl. More typically, R^{IXe} and R^{IXf} are both H. Usually, one of R^{IXg} and R^{IXh} is H and the other is OR^{IXr}, wherein R^{IXr} is selected from H and unsubstituted C₁₋₆ alkyl. More typically, however, one of R^{IXg} and R^{IXh} is H and the other is OH. R^{IXi} is typically unsubstituted C₁₋₆ alkyl, more typically methyl. Typically, R^{IXj} is a group of formula (X). Usually, R^{IXk} is a group of formula (XI). Typically, R^{IXn}, R^{IXo}, R^{IXp} and R^{IXq}, which are the same or different, are independently selected from H and unsubstituted C₁₋₆ alkyl. More typically, each of R^{IXn}, R^{IXo}, R^{IXp} and R^{Ixq} is H. R^{IXm} is typically selected from unsubstituted or substituted C₁₋₁₀ alkyl. More typically, R^{IXm} is an unsubstituted or substituted C₁₋₆ alkyl. R^{IXm} may be, for instance, —CH(CH₃)(C₄H₁₁).

In another embodiment, r is 1 and q is 0. Typically, in this embodiment, R^{IXb} is C₁₋₆ alkyl substituted with a hydroxyl group. More typically, R^{IXb} is CH₂OH. Furthermore, R^{IXa} is typically COOH or an unsubstituted ester. More typically, R^{IXa} is COOH. Usually R^{IXc} and R^{IXd} are independently selected from H and unsubstituted C₁₋₆ alkyl. More typically, however, R^{IXc} and R^{IXd} are both H. Typically, R^{IXe} and R^{IXf} are independently selected from H and unsubstituted C₁₋₆ alkyl. More typically, R^{IXe} and R^{IXf} are both H. Usually, in this embodiment, R^{IXg} and R^{IXh} together form an oxo group. R^{IXi} is typically H. Typically, in this embodiment, R^{IXj} and R^{IXk}, which may be the same or different, are independently selected from H and unsubstituted C₁₋₆ alkyl. More typically, R^{IXj} and R^{IXk} are both H. R^{IXm} is typically, in this embodiment, selected from unsubstituted or substituted C₁₋₆ alkyl. More typically, R^{IXm} is an unsubstituted C₁₋₆ alkyl. R^{IXm} may be, for instance, methyl.

Table 4 shows examples of compounds of formula (IX) which may be employed in the present invention. Such compounds are inhibitors of ceramide biosynthesis. More specifically, compound 67 (Myriocin) is a serine palmitoyltransferase inhibitor and compound 68 (Fumonisin) is a dihydroceramide synthase inhibitor.

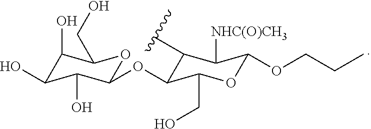

In one embodiment the invention provides the use, in the manufacture of a medicament for the treatment of a glycolipid-mediated autoimmune disease, of a compound of the following formula (XII):

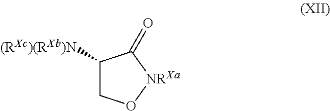

in which:

R^{Xa} is H, substituted or unsubstituted C₁₋₂₀ alkyl, substituted or unsubstituted C₁₋₂₀ alkylene-aryl, substituted or unsubstituted C₁₋₂₀ alkylene-C₃₋₂₀ heteroaryl, substituted or unsubstituted C₁₋₂₀ alkylene-C₃₋₂₅ cycloalkyl, substituted or unsubstituted C₁₋₂₀ alkylene-C₃₋₂₀ heterocyclyl, substituted or unsubstituted C₁₋₂₀ alkylene-O—C₃₋₂₀ heterocyclyl, substituted or unsubstituted aryl, substituted or unsubstituted C₃₋₂₀ heteroaryl, substituted or unsubstituted C₃₋₂₅ cycloalkyl or substituted or unsubstituted C₃₋₂₀ heterocyclyl wherein said C₁₋₂₀ alkyl and C₁₋₂₀ alkylene are optionally interrupted by N(R′), O, S or arylene wherein R′ is H, C₁₋₆ alkyl or aryl; and

R^{Xb} and R^{Xc}, which are the same or different, are independently selected from H, unsubstituted or substituted C₁₋₁₀ alkyl and unsubstituted or substituted aryl;

or a pharmaceutically acceptable salt thereof.

Typically, R^{Xa} is H, substituted or unsubstituted C₁₋₁₀ alkyl or substituted or unsubstituted phenyl. More typically, R^{Xa} is H, unsubstituted C₁₋₆ alkyl or unsubstituted phenyl. Even more typically, R^{Xa} is H.

Typically, R^{Xb} and R^{Xc}, which are the same or different, are independently selected from H, unsubstituted C₁₋₆ alkyl and unsubstituted phenyl. More typically, R^{Xb} and R^{Xc} are both H.

Table 5 shows an example of a compound of formula (XII) which may be employed in the present invention. The compound (compound 69) is an inhibitor of ceramide biosynthesis. More specifically, compound 69 (L-Cycloserine) is a serine palmitoyltransferase inhibitor.

TABLE 5

| Compound | Compound structure (name) |
|---|---|
| 69 | 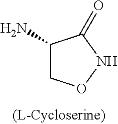 |

In one embodiment, the invention further provides the use, in the manufacture of a medicament for the treatment of a glycolipid-mediated autoimmune disease, of a compound of formula (I), formula (II), formula (III), formula (IV) or formula (V):

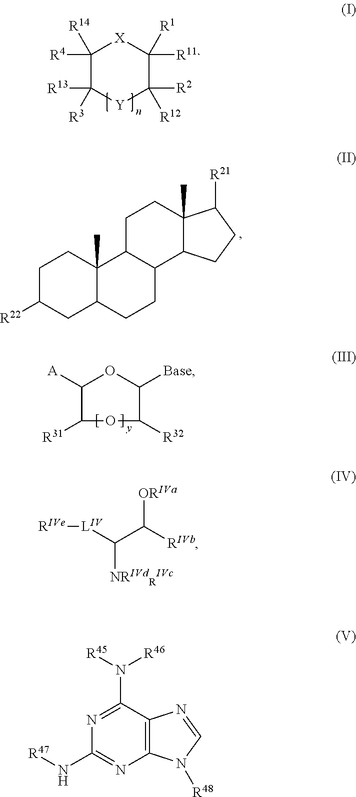

wherein:

X is O, S or NR⁵;

R⁵ is hydrogen, substituted or unsubstituted C₁₋₂₀ alkyl, substituted or unsubstituted C₁₋₂₀ alkylene-aryl, substituted or unsubstituted C₁₋₂₀ alkylene-C₃₋₂₀ heteroaryl, substituted or unsubstituted C₁₋₂₀ alkylene-C₃₋₂₅ cycloalkyl, substituted or unsubstituted C₁₋₂₀ alkylene-C₃₋₂₀ heterocyclyl, substituted or unsubstituted C₁₋₂₀ alkylene-O—C₃₋₂₀ heterocyclyl, substituted or unsubstituted aryl, substituted or unsubstituted C₃₋₂₀ heteroaryl, substituted or unsubstituted C₃₋₂₅ cycloalkyl or substituted or unsubstituted C₃₋₂₀ heterocyclyl, or R⁵ forms, together with R¹, R¹¹, R⁴ or R¹⁴, a substituted or unsubstituted C₁₋₆ alkylene group, wherein said C₁₋₂₀ alkyl and C₁₋₂₀ alkylene are optionally interrupted by N(R′), O, S or arylene wherein R′ is H, C₁₋₆ alkyl or aryl;

n is 0 or 1;

Y is O, S or CR⁶R¹⁶;

R¹, R¹¹, R⁴ and R¹⁴, which may be the same or different, are independently selected from hydrogen, hydroxyl, carboxyl, amino, thiol, substituted or unsubstituted C₁₋₂₀ alkyl, substituted or unsubstituted C₁₋₂₀ alkoxy, substituted or unsubstituted aryloxy, acyl, ester, acyloxy, C₁₋₁₀ alkylamino, di(C₁₋₁₀)alkylamino, amido, acylamido, —O—C₃₋₂₅ cycloalkyl and —O—C₃₋₂₀ heterocyclyl, provided that one of R¹, R¹¹, R⁴ and R¹⁴ may form, together with R⁵, a substituted or unsubstituted C₁₋₆ alkylene group, wherein said C₁₋₂₀ alkyl is optionally interrupted by N(R′), O, S or arylene;

R², R¹², R³, R¹³, R⁶ and R¹⁶, which may be the same or different, are independently selected from hydrogen, hydroxyl, carboxyl, amino, thiol, substituted or unsubstituted C₁₋₂₀ alkyl, substituted or unsubstituted C₁₋₂₀ alkoxy, substituted or unsubstituted aryloxy, acyl, ester, acyloxy, C₁₋₁₀ alkylamino, di(C₁₋₁₀)alkylamino, amido, acylamido —O—C₃₋₂₅ cycloalkyl and —O—C₃₋₂₀ heterocyclyl, wherein said C₁₋₂₀ alkyl is optionally interrupted by N(R′), O, S or arylene;

R²¹ is selected from oxo, -L³⁰-R²³, -L³⁰-C(O)N(H)—R²⁴ and a group of the following formula (VI):

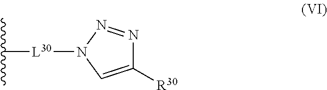

L³⁰ is substituted or unsubstituted C₁₋₂₀ alkylene which is optionally interrupted by N(R′), O, S or arylene;

R²³ is carboxyl, hydroxyl, ester, phosphonate ester, phosphate ester, phosphoric acid and phosphonic acid;

R²⁴ is C₁₋₂₀ alkyl which is unsubstituted or substituted with one or more groups selected from carboxyl, hydroxyl, ester, phosphonate ester, phosphate ester, phosphoric acid and phosphonic acid, wherein said C₁₋₂₀ alkyl is optionally interrupted by N(R′), O, S or arylene;

R³⁰ is C₁₋₂₀ alkyl which is unsubstituted or substituted with one or more groups selected from carboxyl, hydroxyl, ester, amino, phosphonate ester, phosphate ester, phosphoric acid and phosphonic acid, wherein said C₁₋₂₀ alkyl is optionally interrupted by N(R′), O, S or arylene;

R²² is hydroxyl, oxo, acyloxy, phosphoric acid or —OC(O)-alk-C(O)OH, wherein alk is substituted or unsubstituted C₁₋₂₀ alkylene which is optionally interrupted by N(R′), O, S or arylene;

Base is selected from a group of any one of the following formulae (a), (b), (c), (d), (e), (f) and (g):

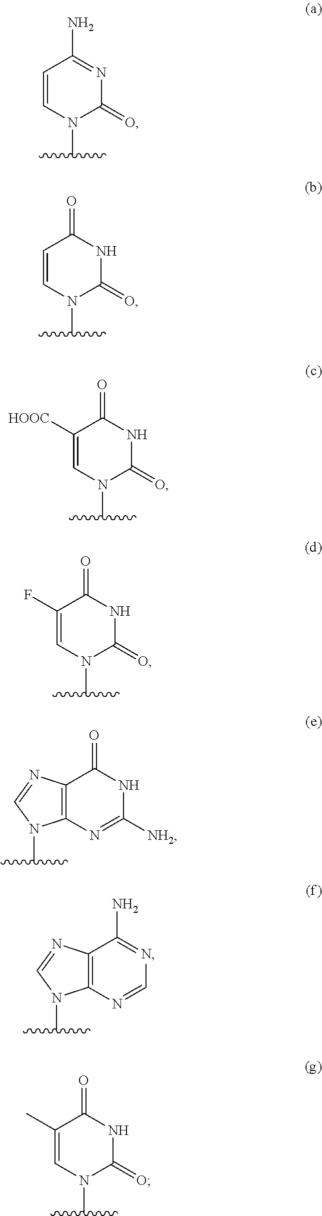

y is 0 or 1;

R³¹ is OH; R³² is H or OH; or, provided that y is 0, R³¹ and R³² together form —O—C(R³³)(R³⁴)—O—, wherein R³³ and R³⁴ are independently selected from H and methyl;

A is substituted or unsubstituted C₁₋₂₀ alkyl, substituted or unsubstituted C₁₋₂₀ alkylene-aryl, substituted or unsubstituted C₁₋₂₀ alkylene-C₃₋₂₀ heteroaryl, substituted or unsubstituted C₁₋₂₀ alkylene-C₃₋₂₅ cycloalkyl or substituted or unsubstituted C₁₋₂₀ alkylene-C₃₋₂₀ heterocyclyl, wherein said C₁₋₂₀ alkyl and C₁₋₂₀ alkylene are optionally interrupted by N(R′), O, S or arylene, wherein R′ is H, C₁₋₆ alkyl or aryl, or A is a group of any one of the following formulae (g) to (k):

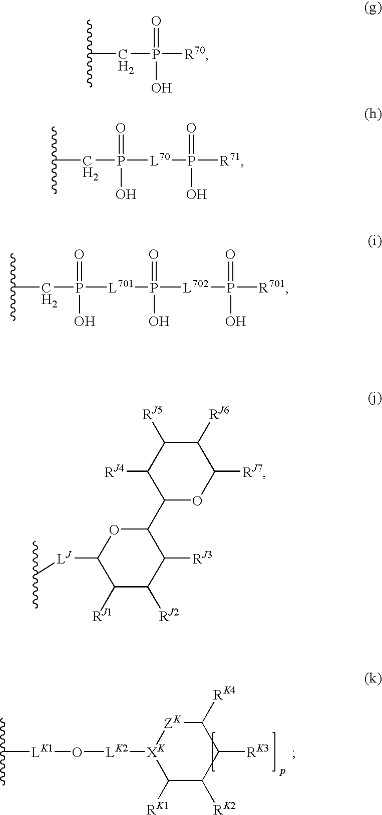

L⁷⁰, L⁷⁰¹ and L⁷⁰² are independently selected from —O—, —C(R³⁵)(R³⁶)— and —NH—, wherein R³⁵ and R³⁶ are independently selected from H, OH and CH₃;

R⁷⁰, R⁷¹ and R⁷⁰¹ are selected from OH, substituted or unsubstituted C₁₋₂₀ alkyl, substituted or unsubstituted C₁₋₂₀ alkoxy, substituted or unsubstituted C₁₋₁₀ alkylamino and -L⁷¹-(X²)ₘ-L⁷²-R⁷²; wherein m is 0 or 1; X² is O, S, —C(R⁴⁵)(R⁴⁶)— or —O—C(R⁴⁵)(R⁴⁶)—, wherein R⁴⁵ and R⁴⁶ are independently selected from H, OH, phosphonic acid or a phosphonic acid salt; L⁷¹ and L⁷² are independently selected from a single bond and substituted or unsubstituted C₁₋₂₀ alkylene, which C₁₋₂₀ alkylene is optionally interrupted by N(R′), O, S or arylene, wherein R′ is H, C₁₋₆ alkyl or aryl; and R⁷² is C₃₋₂₅ cycloalkyl or C₃₋₂₀ heterocyclyl;

L^{J} is substituted or unsubstituted C₁₋₂₀ alkylene;

R^{J1}, R^{J2}, R^{J3}, R^{J4}, R^{J5}, R^{J6} and R^{J7}, which are the same or different, are independently selected from hydrogen, hydroxyl, carboxyl, amino, thiol, substituted or unsubstituted C₁₋₂₀ alkyl, substituted or unsubstituted C₁₋₂₀ alkoxy, substituted or unsubstituted aryloxy, acyl, ester, acyloxy, C₁₋₁₀ alkylamino, di(C₁₋₁₀)alkylamino, amido, acylamido, —N(H)C(O)CH═CH—R^{J8}, —O—C₃₋₂₅ cycloalkyl and —O—C₃₋₂₀ heterocyclyl, wherein said C₁₋₂₀ alkyl is optionally interrupted by N(R′), O, S or arylene, and wherein R^{J8} is substituted or unsubstituted C₁₋₂₀ alkyl;

L^{K1} and L^{K2}, which are the same or different, are independently selected from a single bond and substituted or unsubstituted C₁₋₂₀ alkylene;

X^{K} is N or C(R^{K6}), wherein R^{K6} is H, COOH or ester;

Z^{K} is O or CH(R^{K5});

p is 0 or 1;

R^{K1}, R^{K2}, R^{K3}, R^{K4} and R^{K5}, which are the same or different, are independently selected from hydrogen, hydroxyl, carboxyl, amino, thiol, substituted or unsubstituted C₁₋₂₀ alkyl, substituted or unsubstituted C₁₋₂₀ alkoxy, substituted or unsubstituted aryloxy, acyl, ester, acyloxy, C₁₋₁₀ alkylamino, di(C₁₋₁₀)alkylamino, amido, acylamido, —O—C₃₋₂₅ cycloalkyl and —O—C₃₋₂₀ heterocyclyl, wherein said C₁₋₂₀ alkyl is optionally interrupted by N(R′), O, S or arylene;

R^{IVa} and R^{IVd}, which are the same or different, are independently selected from H, substituted or unsubstituted C₁₋₆ alkyl or substituted or unsubstituted phenyl;

R^{IVb} is H, substituted or unsubstituted aryl, —CH═CHR^{IVf}, or substituted or unsubstituted C₁₋₂₀ alkyl, which C₁₋₂₀ alkyl is optionally interrupted by N(R′), O, S or arylene wherein R′ is H, C₁₋₆ alkyl or aryl;

R^{IVc} is H, substituted or unsubstituted C₁₋₆ alkyl, substituted or unsubstituted phenyl or —C(O)R^{IVc};

R^{IVf} is H or substituted or unsubstituted C₁₋₂₀ alkyl, which C₁₋₂₀ alkyl is optionally interrupted by N(R′), O, S or arylene;

R^{IVg} is H or substituted or unsubstituted C₁₋₂₀ alkyl, which C₁₋₂₀ alkyl is optionally interrupted by N(R′), O, S or arylene;

R^{IVe} is H, hydroxyl, carboxyl, amino, thiol, substituted or unsubstituted C₁₋₂₀ alkyl, substituted or unsubstituted C₁₋₂₀ alkoxy, substituted or unsubstituted aryloxy, acyl, ester, acyloxy, C₁₋₁₀ alkylamino, di(C₁₋₁₀)alkylamino, amido, acylamido, —O—C₃₋₂₅ cycloalkyl, —O—C₃₋₂₀ heterocyclyl, substituted or unsubstituted aryl, substituted or unsubstituted C₃₋₂₀ heteroaryl, substituted or unsubstituted C₃₋₂₅ cycloalkyl or substituted or unsubstituted C₃₋₂₀ heterocyclyl, which C₁₋₂₀ alkyl is optionally interrupted by N(R′), O, S or arylene;

L^{IV} is substituted or unsubstituted C₁₋₂₀ alkylene which C₁₋₂₀ alkylene is optionally interrupted by N(R′), O, S or arylene;

R⁹¹ and R⁹², which are the same or different, are independently selected from H, substituted or unsubstituted C₁₋₂₀ alkyl, substituted or unsubstituted aryl and -L⁹¹-R⁹⁵, wherein L⁹¹ is substituted or unsubstituted C₁₋₂₀ alkylene, wherein said C₁₋₂₀ alkyl and said C₁₋₂₀ alkylene are optionally interrupted by N(R′), O, S or arylene wherein R′ is H, C₁₋₆ alkyl or aryl, and wherein R⁹⁵ is substituted or unsubstituted aryl, amino, C₁₋₁₀ alkylamino or di(C₁₋₁₀)alkylamino;

R⁹³ is -L⁹²-R⁹⁶, wherein L⁹² is a single bond or substituted or unsubstituted C₁₋₂₀ alkylene, which C₁₋₂₀ alkylene is optionally interrupted by N(R′), O, S or arylene, and wherein R⁹⁶ is amido or substituted or unsubstituted aryl; and

R⁹⁴ is H or substituted or unsubstituted C₁₋₂₀ alkyl, which C₁₋₂₀ alkyl is optionally interrupted by N(R′), O, S or arylene;

or a pharmaceutically acceptable salt thereof.

Glycolipid antigens, for instance ganglioside antigens, are associated with a range of clinically distinct pathologies in which antibody or T-cell mediated immunity to the glycolipid leads to disease. It is believed that inhibitors of glycolipid biosynthesis can reduce below a critical threshold or remove the underlying antigenic stimulus of such pathologies by inhibiting the synthesis or expression of the glycolipid antigens. In particular, the inhibition of glycolipid synthesis may reduce epitope formation and hence reduce anti-glycolipid mediated tissue damage and also reduce the effector functions of the autoimmune response such as autoreactive T-cells and B-cells. Accordingly, glycolipid-mediated autoimmune diseases can be treated with an inhibitor of glycolipid biosynthesis in accordance with the present invention.

The term “glycolipid-mediated autoimmune disease”, as used herein, means a disease in which antibody-mediated or T-cell-mediated immunity to a glycolipid leads to disease or contributes to pathology.

Examples of glycolipid-mediated autoimmune diseases include, but are not limited to, the following conditions, all of which have circulating anti-glycolipid autoantibodies (see Misasi et al. 1997, Diabetes/metabolism reviews, Vol. 13 No. 2, 163-179 and references therein):

### Autoimmune Peripheral Neuropathies:

Guillain-Barré syndrome (GBS)

Variants of Guillain-Barré syndrome, for instance Miller Fisher syndrome (MFS)

GBS with opthalmoplegia

Cranial nerve variants of Miller Fisher syndrome, for instance Bickerstaffs brainstem encephalitis and

Acute motor axonal neuropathy (AMAN)

Motor neuropathy (J Neurol. 1991 December; 238(8):447-51)

Motor neuropathy with multifocal conduction blocks (J Neuroimmunol. 1991 June; 32(3):223-30)

Lower motor neuron syndromes (Ann Neurol. 1990 March; 27(3):316-26)

Chronic inflammatory demyelinating polyneuropathy (CIDP) (J Neuroimmunol. 1996 October; 70(1):1-6)

Multifocal CIDP (Lewis-Summer syndrome)

Acute inflammatory demyelinating polyneuropathy (AIDP)

Subacute inflammatory demyelinating polyneuropathy (SIDP)

Sensory neuropathies (anti-GD3, GD1b, GD1a, GQ1b gangliosides or sulfatides) (Steck A J Rev Neurol (Paris). 1996 May; 152(5):400-4)

Multifocal Motor Neuropathy (MMN)

Multifocal motor demyelinating neuropathy (reviewed in: Semin Neurol. 1998; 18(1):73-81)

Mixed motor sensory neuropathy

Multifocal motor sensory neuropathy (MMSN)

Chronic idiopathic sensory ataxic neuropathy (Rev Neurol (Paris). 2001 May; 157(5):517-22)

Chronic recurrent polyneuropathy (Rinsho Shinkeigaku. 1995 October; 35(10):1131-6. Review. Japanese)

Acute Motor Sensory Axonal neuropathy (AMSAM)

Sciatica (Spine. 2002 Feb. 15; 27(4):380-6)

Autoimmune mononeuritis multiplex

Acute relapsing sensory-dominant polyneuropathy associated with anti-GQ1b antibody (Rinsho Shinkeigaku. 1994 September; 34(9):886-91. Japanese)

Amyotrophic lateral sclerosis (Meininger V. 1991 Neurology 41: 315)

Diabetic neuropathy

Acute panautonomic neuropathy

Bell's palsy (J Neurol Sci. 1975 September; 26(1):13-20)

Acute opthalmoparesis

### Autoimmune Central Neuropathies:

Multiple sclerosis (anti-sulfatide, ganglioside) (Kasai N, J Neurol Sci. 1986 August; 75(1):33-42., Nat. Med. 2006, 12:138-43)

Transverse myelitis

Optic neuritis

Chronic myelinic neuropathy with IgM gammopathy (anti-MAG) (Steck A J Rev Neurol (Paris). 1996 May; 152(5):400-4)

Cryptogenic partial epilepsies (Epilepsia. 1996 October; 37(10):922-6)

Partial oculomotor nerve palsy (J Pediatr. 2000 September; 137(3):425-6)

Isolated cranial neuropathy (J Neurol Sci. 2004 Oct. 15; 225(1-2):51-5)

Autoimmune cerebellar disease (Neurology 2003 May 27; 60(10):1672-3; Neurology, 2004 Feb. 10; 62(3):528)

Acute Disseminated Encephalomyelitis (ADEM) (J Neurol. 2005 May; 252(5):613-4. Epub 2005 Mar. 22)

Stiff-man syndrome (Moersch-Woltmann syndrome)

Bickerstaff's brainstem encephalopathy

### Connective Tissue Diseases:

Systemic lupus erythamatosus (SLE) (Endo T, Scott D D 1984 Immunol 132:1793-1797)

Discoid lupus

Scleroderma

Morphoea

CREST (calcinosis, raynaud's syndrome, esophageal dysmotility, slerodactyl), telangiectasia)

Mixed connective tissue disease

Relapsing polychondritis

Sjogren's syndrome (Clin Rheumatol. 2003 September; 22(3):256-8., Psychoneuroendocrinology 1992 November; 17(6):593-8)

Primary fibromyalgia syndrome (Psychoneuroendocrinology 1992 November; 17(6):593-8)

Autoimmunity as Complications of Drug Therapies, such as Therapies with (Reviewed in: Curr Opin Neurol 2002; 15:633-638):

Tumor necrosis factor-α (TNFα) blockers (Neurology 2005; 64:1468-1470)

Interferon-α

Tacrolimus (FK506)

Cyclosporine A

Suramin

Cisplatin

Zimeldine

Captopril (Postgrad Med J. 1987 March; 63(737):221-2)

Danazol

Gold (Scand J Rheumatol. 1982; 11(2):119-20)

Penicillamine (Aust N Z J. Med. 1984 February; 14(1):50-2)

Streptokinase (Med J Aust. 1995 Feb. 20; 162(4):214-5)

Anistreplase (Chest. 1994 April; 105(4):1301-2)

Autoimmune Complications of Vaccines such as:

Influenza vaccination (JAMA. 2005 Dec. 7; 294(21):2720-5. JAMA 2004 Nov. 24; 292(20):2478-81, BMJ. 2003 Mar. 22; 326(7390):620)

Menactra meningococcal conjugate vaccine

‘Psycho-neuro-endocrinological autoimmune diseases’ (Eur J Med Res. 1995 Oct. 16; 1(1):21-6):

Fibromyalgia syndrome (Eur J Med. Res. 1995 Oct. 16; 1(1):21-6)

Chronic fatigue syndrome (Eur J Med Res. 1995 Oct. 16; 1(1):21-6)

### Autoimmune Vasculitides:

Behçet's disease

### Autoimmune Thyroiditis:

Hashimoto's thyroiditis

Graves' disease

### Non-Vascular Dementias:

Alzheimer's disease

### Autoimmune Endocrinopathies:

Insulin-dependent (type I) diabetes mellitus

Primary adrenal failure

### Late Complications of Infective Tick Borne Diseases, Such as:

Neuroborreliosis (Oschmann P. Infection. 1997 September-October; 25(5):292-7)

Acute Disseminated Encephalomyelitis (ADEM) (J Neurol. 2005 May; 252(5):613-4. Epub 2005 Mar. 22)

### Autoimnmune Arthritides:

Rheumatoid arthritis (RA)

Still's disease

### Autoimmune Gastrointestinal Diseases:

Coeliac disease (anti-GM1, anti-GD1b and anti-GQ1b) (Dig Liver Dis. 2006. March; 38(3):183-7. Epub 2006 Feb. 7. Neurology 2003 May 27; 60(10):1672-3. Neurology. 2004 Feb. 10; 62(3):528.)

Crohn's disease (Acta Leprol. 1989; 7 Suppl 1:138-40.)

Ulcerative colitis

Pernicious anoemia

### Autoimmune Clotting Disorders:

Idiopathic thrombocytopenic purpura (Br J Haematol. 1987 September; 67(1):103-8.)

### Glomerulonephritides Such as:

IgA Nephropathy (Nippon Jinzo Gakkai Shi. 1991 July; 33(7):635-42. Japanese.)

### Ear Disorders:

Meniere's disease (Biochim Biophys Acta. 2000 Jun. 15; 1501(2-3):81-90)

Autoimmune inner ear disease (anti-sulfoglucuronosyl glycolipids) (J Neuroimmunol. 1998 Apr. 15; 84(2):111-6)

Acute opthalmoparesis

### Further Glycolipid-Mediated Autoimmune Diseases:

Autoimmune hemolytic anemia (Rinsho Shinkeigaku. 1995 October; 35(10):1131-6. Review. Japanese)

Autoimmune hepatitis (Rinsho Shinkeigaku. 1994 September; 34(9):886-91. Japanese)

Typically, the glycolipid-mediated autoimmune disease is a glycolipid-mediated autoimmune disease other than Alzheimer's disease.

Typically, the glycolipid-mediated autoimmune disease is a glycolipid-mediated autoimmune disease other than multiple sclerosis (MS).

In one embodiment, the glycolipid-mediated autoimmune disease is a glycolipid-mediated autoimmune disease other than multiple sclerosis (MS) and other than Alzheimer's disease.

Guillain-Barré syndrome (GBS) and Miller Fisher syndrome (MFS) are post-infectious autoimmune diseases. GBS is an acute, paralysing, inflammatory peripheral nerve disease and is the most frequent cause of acute flacid paralysis. In fact, GBS is the world's leading cause of neuromuscular paralysis, affecting 1 in a 1000 individuals worldwide at some point in their lives, and leaving 20% disabled or dead. Chronic forms of the syndrome also exist. The economic and societal costs, and personal suffering are substantial. A single, severely affected case can cost >£1M in acute care, rehabilitation, and lifetime disability benefits. Current treatment comprises non-specific immunotherapy and is limited in efficacy. Considering the high incidence of GBS and high healthcare costs, it is surprising how little effort has been invested in rational therapy design. GBS is also known as Landry-Guillain-Barré syndrome, acute idiopathic polyneuritis, infectious polyneuritis and acute inflammatory polyneuropathy. GBS and chronic counterparts are caused by inflammation of the peripheral nervous system, leading to nerve conduction failure, manifested clinically as paralysis. Through a breakdown of immunological tolerance, antibodies are mistakenly formed against sugar structures on the surface of infectious agents preceding GBS, and these antibodies inadvertently attack identical sugars structures carried by gangliosides on the surface of peripheral nerves. This triggers a destructive cascade of inflammatory molecules that overwhelms natural immune defences and severely damages nerves. The myelin and axonal target molecules for this autoantibody attack have been identified as nerve gangliosides in a significant proportion of cases. In human disease, anti-GM1, -GD1a, -GM1b and -GalNAcGD1a antibodies are associated with axonal forms of GBS, and anti-GQ1b, -GT1a and -GD1b antibodies with acute and chronic ataxic neuropathies. Anti-GM1 antibodies are also associated with acute and chronic demyelinating phenotypes, with or without concomitant axonal disease. Thus GBS is characterised by autoantibodies against peripheral nerves leading to deposition of complement components and the development of acute motor axonal neuropathy (AMAN). These autoantibodies are directed to cell surface gangliosides. Most GBS and MFS patients have had a prior infection with Campylobacter jejuni. The adaptive immune response toward lipooligosaccharide components of C. jejuni leads to the formation of antibodies that cross react with ‘self’ glycolipids. In particular, the clinical manifestation of the autoimmune response is influenced by the different strain of infecting C. jujeni. For example, the C. jujeni strain (CF90-26) carries a lipooligosaccharide that contains both the Galβ1-3GalNAcβ1-4[NeuNAcα2-3]Galβ and NeuNAcα2-3Galβ1-3GalNAcβ1-4-[NeuNAcα2-3]Galβ carbohydrate motifs, which can lead to the formation of autoantibodies against the ‘self’ ganglioside, GM1 and GD1a, respectively. Further infecting strains contain lipooligosaccharides that mimic GT1a and GD1c. These can lead to the formation of autoantibodies, anti-GT1a and anti-GD1c. Some of these antibodies also cross-react with GQ1b, which is present on oculomotor nerves and primary sensor neurons, leading to the Miller Fisher variation of GBS characterised by opthalmoplegia and ataxia. GBS may also be triggered by other infections such as chlamydia infections, cytomegalovirus infections, mononucleosis and mycoplasma pneumonia. Existing treatments for GBS are (1) plasma exchange (PE) (Harel M, et al. Clin Rev Allergy Immunol. 2005 December; 29(3):281-7; Hughes R A et al. Lancet. 2005 Nov. 5; 366(9497):1653-66).

GBS and its variants, such as MFS, can be induced by drug treatment. Fagius, J. et al., “Guillain-Barré syndrome following zimeldine treatment”, Journal of Neurology, Neurosurgery, and Psychiatry, 1985, Vol 48, 65-69 reviews thirteen cases of Guillain-Barré syndrome, all occurring with a similar relationship to recent commencement of treatment with the antidepressive drug zimeldine. The risk of developing Guillain-Barré syndrome was increased about 25-fold among patients receiving zimeldine, as compared with the natural incidence of the disorder. The cases described provide strong evidence that GBS or variants of GBS may occur as a specific, probably immunologically mediated, complication of drug therapy. GBS and its variants, such as MFS, may also occur as a complication of immunisation for influenza (JAMA. 2005 Dec. 7; 294(21):2720-5. JAMA 2004 Nov. 24; 292(20):2478-81, BMJ. 2003 Mar. 22; 326(7390):620).

Inhibitors of glycolipid biosynthesis reduce cell surface glycolipids and can thus be used to treat Guillain-Barré and Miller-Fisher syndromes. The reduction in the formation of glycolipid epitopes leads to the reduction in glycolipid-autoantibody complexes.

Typically, therefore, the glycolipid-mediated autoimmune disease is Guillain-Barré syndrome, or a variant thereof.

Insulin-dependent (type I) diabetes mellitus is caused by the destruction by the immune system of insulin-producing pancreatic islet cells. Although there are some protein epitopes (such as GAD65 and islet tyrosinase phosphatase IA-2), numerous glycolipids, including gangliosides (such as GT3, GD3 and GM2-1) and sulfatides, are recognised by autoantibodies in the phase leading to clinical manifestation of the disease. During this phase, the so-called islet cell autoantibodies (ICA) contribute to the destruction of the pancreatic islet cells. The GM2-1 is particularly localized on the pancreatic islet cells and acts to focus the autoantibodies to those cells. In animal models of diabetes (NOD and BB mice) the destruction of the islet β-cells could be influenced by their metabolic status. Islet destruction and the onset of diabetes could be prevented by the down-regulation of the b-cell metabolism. Exogenous insulin administration reduced the expression of the ganglioside autoantigen, GM2-1. In contrast, the levels of GM3 and GD3 were not reduced, indicating that GM2-1 is a significant autoantigen in autoimmune diabetes. (Gotfredsen C F et al 1985 Diabetologia 28:933-935; Atkinson M A et al 1990 Diabetes, 39:933-937; Appel M C et al. 1989 diabetalogia 32:461 A). In addition, patients with insulin dependent diabetes have elevated anti-sulfatide autoantibodies. (Autoimmunity. 2002 November; 35(7):463-8). Sulfotransferase inhibitors, for instance the sulfotransferase inhibitors described in Armstrong, J. I. et al. Angew. Chem. Int. Ed. 2000, 39, No. 7, 1303-1306 and references therein, may be effective in reducing the levels of those anti-sulfatide autoantibodies.

Inhibitors of glycolipid biosynthesis reduce the cell surface expression of autoantigens and can thus be used to treat Type 1 Diabetes Mellitus.

Multiple Sclerosis (MS) is a disease of the central nervous system with a significant autoimmune component. Both T-cell and antibody mediated immunity has been described in the pathogenesis of MS. Lipids comprise the majority of the myelin sheath (70%). Recent evidence shows that neuronal expression of GM2, GD1a, GD1b, GD3 and GT1b were all expressed at an elevated level in cells which would, in normal CNS, express these antigens at low levels or not at all (Marconi et al 2005, J.Neuroimmunology 170:115). Consistent with this observation, much of the antibody mediated autoimmunity is known to be directed against myelin glycolipids. Specifically, microarray analysis has revealed an MS-dependent increase in antibody reactivity to sulfatides, sphingomyelin, and oxidized lipids, including 3b-hydroxy-5a-cholestan-15-one, and 1-palmityl-2-(9′-oxo-nonanoyl)-sn-glycero-3-phophocholine (Kanter J L et al. 2006 Nat. Med. 12:1:138-143). In addition to antibody recognition of glycolipids, T-cell recognition of lipid antigens, restricted by the CD1 system has been reported at the site of lesions in MS. CD1 expression is increased in CNS lesions in both MS and EAE. As in the case of Guillain-Barré syndromes, reactivity to lipopolysaccharide (LPS) is elevated, indicating a potential role for mimicry or cross reactivity of these antibodies. Furthermore, transfer of sulfatide-specific antibodies, or immunization with sulfatide (with an immunogenic adjuvant) increases disease progression in the experimental autoimmune encephalomyelitis (EAE) animal model of MS. These data all indicate that antibodies directed to the lipids of myelins may play a causative role in neuropathology. Thus direct antibody mediated neuropathy (as well as CD1-mediated immune activation) may be improved by the reduction of autoreactive lipid epitopes.

Inhibitors of glycolipid biosynthesis reduce cell surface expression of autoantigens and can thus be used to treat MS.

Rheumatoid Arthritis (RA) is an autoimmune disease that involves inflammation and destruction of connective joint tissue. There is much evidence for an association between RA and anti-glycolipid reactivity. However, the role of anti-glycolipid reactivity remains unclear (Zeballos et al. 1994, J.Clin.Lab.Anal 8(6):378). Nonetheless, anti-glycolipid reactivity is likely to contribute to secondary autoimmunity that may be moderated by reduction of reactive gangliosides. For example, anti-ganglioside reactivity is found in RA patients who develop additional, neuronal pathology. Thus immunological deregulation resulting from RA may predispose towards further, “bystander” anti-ganglioside reactivities and subsequent neuronal damage (McCombe et al. 2000 J. Clin. Neurosci.7(3)209).

Therefore, it is believed that reduction of reactive glycolipids using inhibitors of glycolipid biosynthesis can reduce the extent of peripheral neuropathy.

An inhibitor of glycolipid biosynthesis, for use in accordance with the present invention, can be administered in a variety of dosage forms, for example orally such as in the form of tablets, capsules, sugar- or film-coated tablets, liquid solutions or suspensions or parenterally, for example intramuscularly, intravenously or subcutaneously. The compound may therefore be given by injection or infusion.

The inhibitor of glycolipid biosynthesis may be presented for administration in a liposome. Thus, the inhibitor may be encapsulated or entrapped in the liposome and then administered to the patient to be treated. Active ingredients encapsulated by liposomes may reduce toxicity, increase efficacy, or both, Notably, liposomes are thought to interact with cells by stable absorption, endocytosis, lipid transfer, and fusion (R. B. Egerdie et al., 1989, J. Urol. 142:390). Drug delivery via liposomes is a well-explored approach for the delivery of iminosugars. Costin G E, Trif M, Nichita N, Dwek R A, Petrescu S M Biochem Bioplys Res Commun. 2002 May 10; 293(3):918-23 describes the use of liposomes composed of dioleoylphosphatidylethanolamine and cholesteryl hemisuccinate for the delivery of NB-DNJ. In that study, the use of liposomes reduced the required dose of NB-DNJ by a factor of 1000, indicating that liposomes are efficient carriers for iminosugar delivery in mammalian cells.

The dosage depends on a variety of factors including the age, weight and condition of the patient and the route of administration. Daily dosages can vary within wide limits and will be adjusted to the individual requirements in each particular case. Typically, however, the dosage adopted for each route of administration when a compound is administered alone to adult humans is 0.0001 to 50 mg/kg, most commonly in the range of 0.001 to 10 mg/kg, body weight, for instance 0.01 to 1 mg/kg. Such a dosage may be given, for example, from 1 to 5 times daily. For intravenous injection a suitable daily dose is from 0.0001 to 1 mg/kg body weight, preferably from 0.0001 to 0.1 mg/kg body weight. A daily dosage can be administered as a single dosage or according to a divided dose schedule.

Typically a dose to treat human patients may range from about 0.1 mg to about 1000 mg of a compound for use in accordance with the invention, more typically from about 10 mg to about 1000 mg of a compound for use in accordance with the invention. A typical dose may be about 100 mg to about 300 mg of the compound. A dose may be administered once a day (QID), twice per day (BID), or more frequently, depending on the pharmacokinetic and pharmacodynamic properties, including absorption, distribution, metabolism, and excretion of the particular compound. In addition, toxicity factors may influence the dosage and administration regimen. When administered orally, the pill, capsule, or tablet may be ingested daily or less frequently for a specified period of time. The regimen may be repeated for a number of cycles of therapy.

A compound is formulated for use as a pharmaceutical composition also comprising a pharmaceutically acceptable carrier or diluent. The compositions are typically prepared following conventional methods and are administered in a pharmaceutically suitable form. The compound may be administered in any conventional form, for instance as follows:

A) Orally, for example, as tablets, coated tablets, dragees, troches, lozenges, aqueous or oily suspensions, liquid solutions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavouring agents, colouring agents and preserving agents in order to provide pharmaceutically elegant and palatable preparations.

Tablets contain the active ingredient in admixture with non-toxic pharmaceutically acceptable excipients which are suitable for the manufacture of tablets. These excipients may be for example, inert diluents, such as calcium carbonate, sodium carbonate, lactose, dextrose, saccharose, cellulose, corn starch, potato starch, calcium phosphate or sodium phosphate; granulating and disintegrating agents, for example, maize starch, alginic acid, alginates or sodium starch glycolate; binding agents, for example starch, gelatin or acacia; lubricating agents, for example silica, magnesium or calcium stearate, stearic acid or talc; effervescing mixtures; dyestuffs, sweeteners, wetting agents such as lecithin, polysorbates or lauryl sulphate. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed. Such preparations may be manufactured in a known manner, for example by means of mixing, granulating, tableting, sugar coating or film coating processes.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is present as such, or mixed with water or an oil medium, for example, peanut oil, liquid paraffin, or olive oil.

Aqueous suspensions contain the active materials in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides for example polyoxyethylene sorbitan monooleate.

The said aqueous suspensions may also contain one or more preservatives, for example, ethyl or n-propyl p-hydroxybenzoate, one or more colouring agents, such as sucrose or saccharin.

Oily suspension may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavouring agents may be added to provide a palatable oral preparation. These compositions may be preserved by this addition of an antioxidant such as ascorbic acid. Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavouring and colouring agents, may also be present.

Pharmaceutical compositions for use in accordance with the invention may also be in the form of oil-in-water emulsions. The oily phase may be a vegetable oil, for example olive oil or arachis oils, or a mineral oil, for example liquid paraffin or mixtures of these. Suitable emulsifying agents may be naturally-occurring gums, for example gum acacia or gum tragacanth, naturally occurring phosphatides, for example soy bean lecithin, and esters or partial esters derived from fatty acids an hexitol anhydrides, for example sorbitan mono-oleate, and condensation products of the said partial esters with ethylene oxide, for example polyoxyethylene sorbitan monooleate. The emulsion may also contain sweetening and flavouring agents. Syrups and elixirs may be formulated with sweetening agents, for example glycerol, sorbitol or sucrose. In particular a syrup for diabetic patients can contain as carriers only products, for example sorbitol, which do not metabolise to glucose or which only metabolise a very small amount to glucose.

Such formulations may also contain a demulcent, a preservative and flavouring and coloring agents;

B) Parenterally, either subcutaneously, or intravenously, or intramuscularly, or intrasternally, or by infusion techniques, in the form of sterile injectable aqueous or oleaginous suspensions. This suspension may be formulated according to the known art using those suitable dispersing of wetting agents and suspending agents which have been mentioned above. The sterile injectable preparation may also be a sterile injectable solution or suspension in a non-toxic paternally-acceptable diluent or solvent, for example as a solution in 1,3-butane diol.

Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition fatty acids such as oleic acid find use in the preparation of injectables;

C) By inhalation, in the form of aerosols or solutions for nebulizers;

D) Rectally, in the form of suppositories prepared by mixing the drug with a suitable non-irritating excipient which is solid at ordinary temperature but liquid at the rectal temperature and will therefore melt in the rectum to release the drug. Such materials are cocoa butter and poly-ethylene glycols;

E) Topically, in the form of creams, ointments, jellies, collyriums, solutions or suspensions.

F) Vaginally, in the form of pessaries, tampons, creams, gels, pastes, foams or spray formulations containing in addition to the active ingredient such carriers as are known in the art to be appropriate.

The present invention is further illustrated in the Examples which follow:

### EXAMPLES

### Example 1

### Epitope Reduction of Ganglioside Antigens by N-butyl Deoxynojirimycin (NB-DNJ)

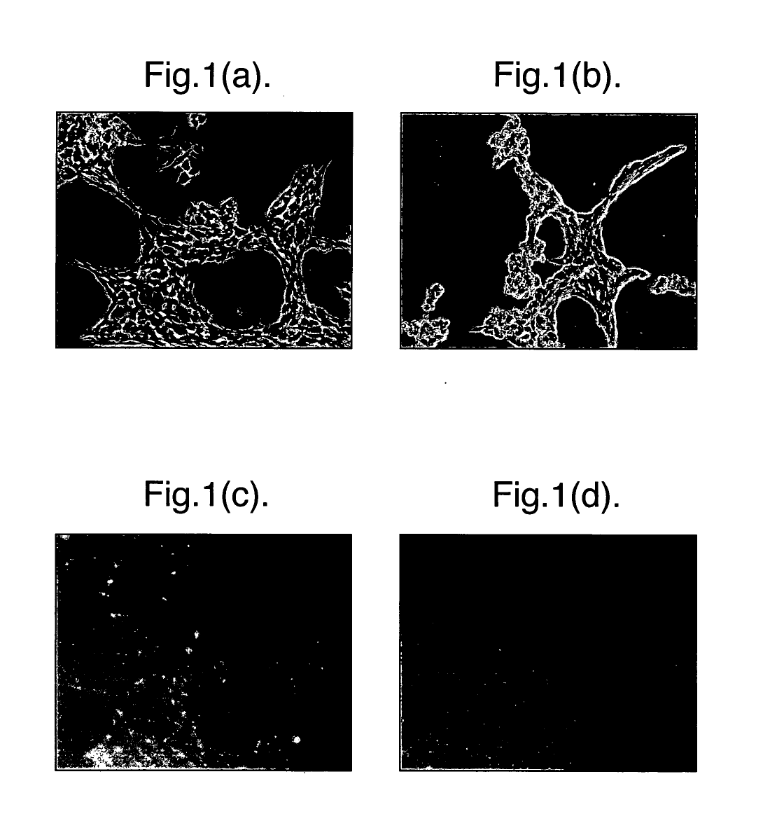

Human neuroblastoma cells were grown in Dulbecco's Modified Medium with Fetal calf serum (10%) and non-essential amino acids (1%), in the presence of penicillin and streptomycin, at 37° C. and 5% CO₂. At 50% confluence, fresh media was added (either with or without 500 μM NB-DNJ) and incubated for a further 4 days. GM1 on the cell surface was detected by addition of polyclonal rabbit anti-GM1 IgG (CalBioChem 1:100). Antibody binding was detected using fluorescent (Alexa-Fluor 488) anti-Rabbit Ab IgG (1:1000). Images (shown in FIG. 1) were collected using a Nikon TE2000-U fluorescent microscope.

### Example 2

### Tablet Composition

Tablets, each weighing 0.15 g and containing 25 mg of an inhibitor of glycolipid biosynthesis, for use in accordance with the invention, are manufactured as follows:

### Composition for 10,000 Tablets

| | |
|---|---|
| Active compound | (250 g) |
| Lactose | (800 g) |
| Corn starch | (415 g) |
| Talc powder | (30 g) |
| Magnesium stearate | (5 g) |

The active compound, lactose and half of the corn starch are mixed. The mixture is then forced through a sieve 0.5 mm mesh size. Corn starch (10 g) is suspended in warm water (90 ml). The resulting paste is used to granulate the powder. The granulate is dried and broken up into small fragments on a sieve of 1.4 mm mesh size. The remaining quantity of starch, talc and magnesium is added, carefully mixed and processed into tablets.

### Example 3

### Injectable Formulation

### Formulation A

| | |
|---|---|
| Active compound | 200 mg |
| Hydrochloric Acid Solution 0.1M or | 4.0 to 7.0 |
| Sodium Hydroxide Solution 0.1M q.s. to pH | |
| Sterile water q.s. to | 10 ml |

The inhibitor of glycolipid biosynthesis, for use in accordance with the invention, is dissolved in most of the water (35° 40° C.) and the pH adjusted to between 4.0 and 7.0 with the hydrochloric acid or the sodium hydroxide as appropriate. The batch is then made up to volume with water and filtered through a sterile micropore filter into a sterile 10 ml amber glass vial (type 1) and sealed with sterile closures and overseals.

### Formulation B

| | | |
|---|---|---|
| Active Compound | 125 | mg |
| Sterile, Pyrogen-free, pH 7 Phosphate | 25 | ml |
| Buffer, q.s. to | | |
| Active compound | 200 | mg |
| Benzyl Alcohol | 0.10 | g |
| Glycofurol 75 | 1.45 | g |
| Water for injection q.s to | 3.00 | ml |

The active compound is dissolved in the glycofurol. The benzyl alcohol is then added and dissolved, and water added to 3 ml. The mixture is then filtered through a sterile micropore filter and sealed in sterile 3 ml glass vials (type 1).

### Example 4

### Syrup Formulation

| | | |
|---|---|---|
| Active compound | 250 | mg |
| Sorbitol Solution | 1.50 | g |
| Glycerol | 2.00 | g |
| Sodium benzoate | 0.005 | g |
| Flavour | 0.0125 | ml |
| Purified Water q.s. to | 5.00 | ml |

The inhibitor of glycolipid biosynthesis, for use in accordance with the invention, is dissolved in a mixture of the glycerol and most of the purified water. An aqueous solution of the sodium benzoate is then added to the solution, followed by addition of the sorbitol solution and finally the flavour. The volume is made up with purified water and mixed well.

### Example 5

### Protection of PC12 Cells from Complement-Mediated Lysis by Pre-Treatment with N-Butyl Deoxynojirimycin (NB-DNJ): Dose and Time-Dependency

Anti-ganglioside antibody-mediated GBS was modelled in the mouse and the central role for gangliosides in targeting antibodies to the nerve membranes was proven. Using various glycosyltransferase knockout mice, it was established that this targeting is highly dependent upon not only the presence, but also the concentration of gangliosides in the nerve membranes.

Therefore, reducing nerve ganglioside levels below a critical threshold using an inhibitor of glycolipid biosynthesis (for instance an imino sugar such as NB-DNJ) might sufficiently deplete the antigenic target to a level at which anti-ganglioside antibodies no longer bind and are ineffective in inducing nerve injury.

Based upon this hypothesis a pilot study has been performed using an in vitro model of GBS. PC12 cells express gangliosides and are susceptible to GBS antibody mediated lysis. The pilot study addresses the question of whether pre-treatment with NB-DNJ protects PC12 cells from GBS antibody mediated lysis.

PC12 Cells can be Protected from Complement-Mediated Lysis by Pre-Treatment with NB-DNJ: Dose and Time Dependency

PC12 cells were grown under standard undifferentiated conditions with and without a range of NB-DNJ concentrations (1, 5, 10, 50, 100 and 500 μM). Cells were grown in flasks for immunocytology and analytical studies on ganglioside levels and also in 96-well tissue culture plates for antibody mediated lysis studies. Time points examined were day 0, 1, 2 and 3, and 2 and 3 days post-compound wash out.

### (i) Immunocytology Studies on Ganglioside Levels

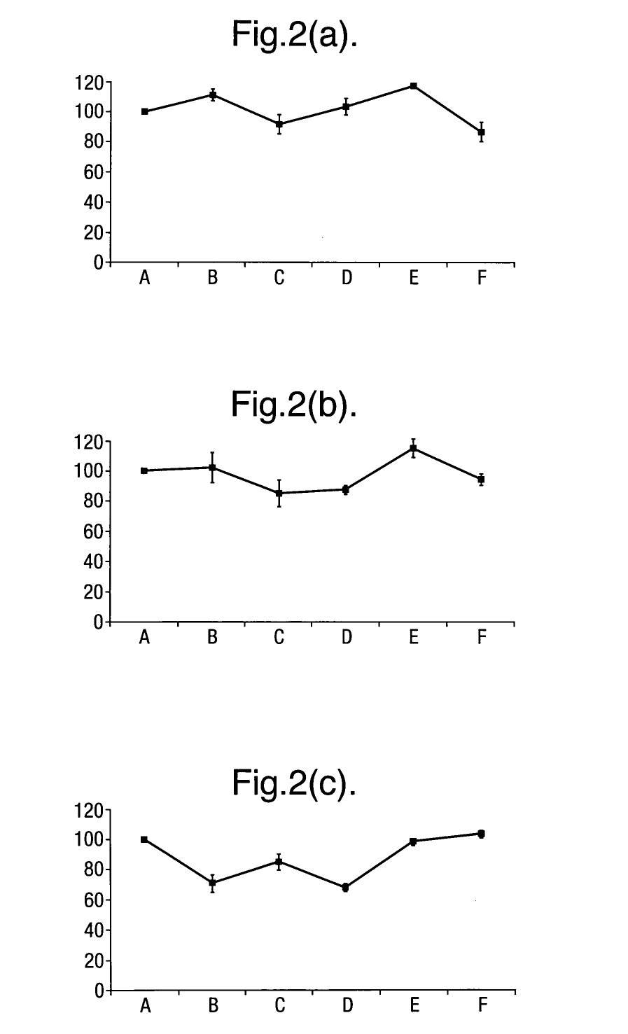

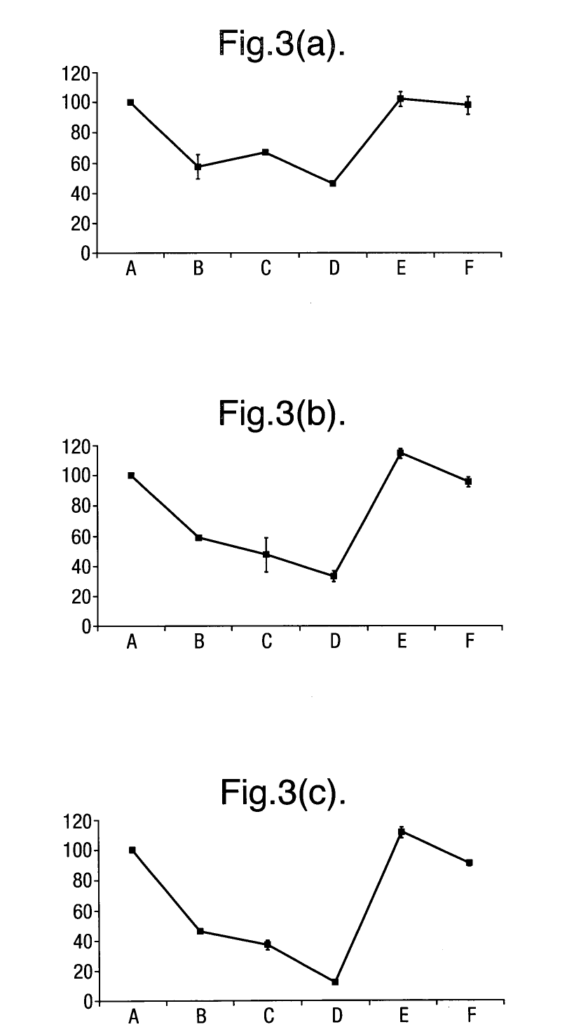

PC12 cells were grown in DMEM containing 7.5% FCS and 7.5% horse serum. The medium was supplemented with 0, 1, 5, 10, 50, 100 or 500 μM NB-DNJ and the cells cultured for 3 days. The media was then replaced with DMEM without NB-DNJ and the cells cultured for a further 3 days. At days 0, 1, 2, 3 and days 2 and 3 post NB-DNJ treatment, cells were harvested and ganglioside levels assessed by analysing anti-ganglioside antibody binding using flow cytometry. Briefly, 1×10⁵ cells were stained with 10 μg/ml of a murine anti-GT1b mAb for 1 hour at room temperature. Binding was then detected by a FITC labelled antibody with specificity for mouse IgG. Binding is expressed in FIGS. 2 and 3 as the mean fluorescence of triplicate experiments. To allow any reduction in ganglioside levels to be clearly visualised at each time point, the mean fluorescence is shown as a percentage of that at day 0. Error bars indicate sem.

At 50 μM and higher NB-DNJ concentrations a significant time dependent reduction in anti-ganglioside antibody binding was observed (FIGS. 2 and 3). The optimal concentrations of NB-DNJ were 100 μM and 500 μM as they achieved a 70% and 90% reduction in antibody binding respectively, following 3 days of exposure to the drug. Wash out of the compound led to the anti-ganglioside antibody binding returning to the levels of untreated cells by day 3.

### (ii) Antibody Mediated Lysis Studies

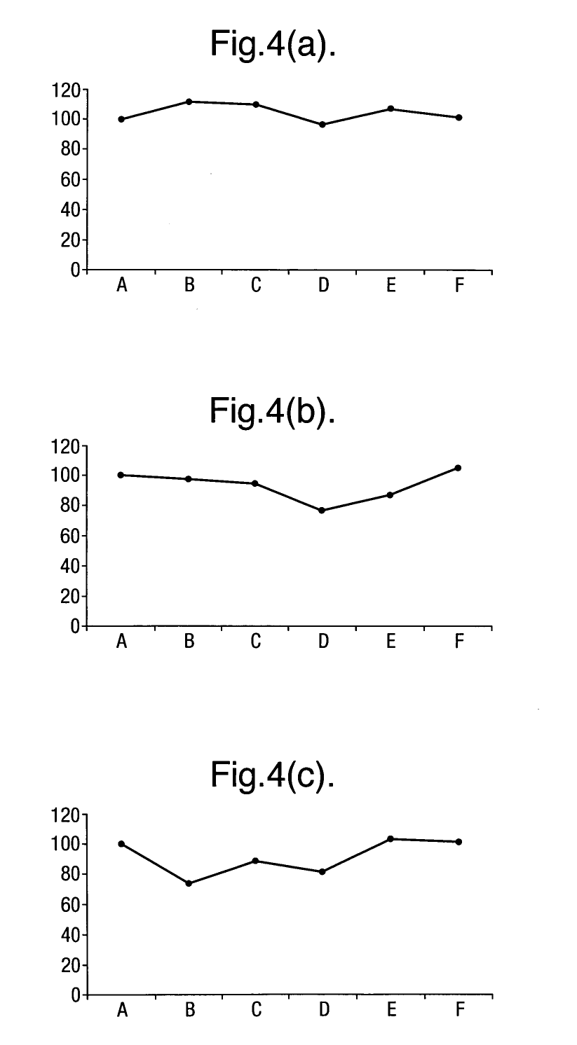

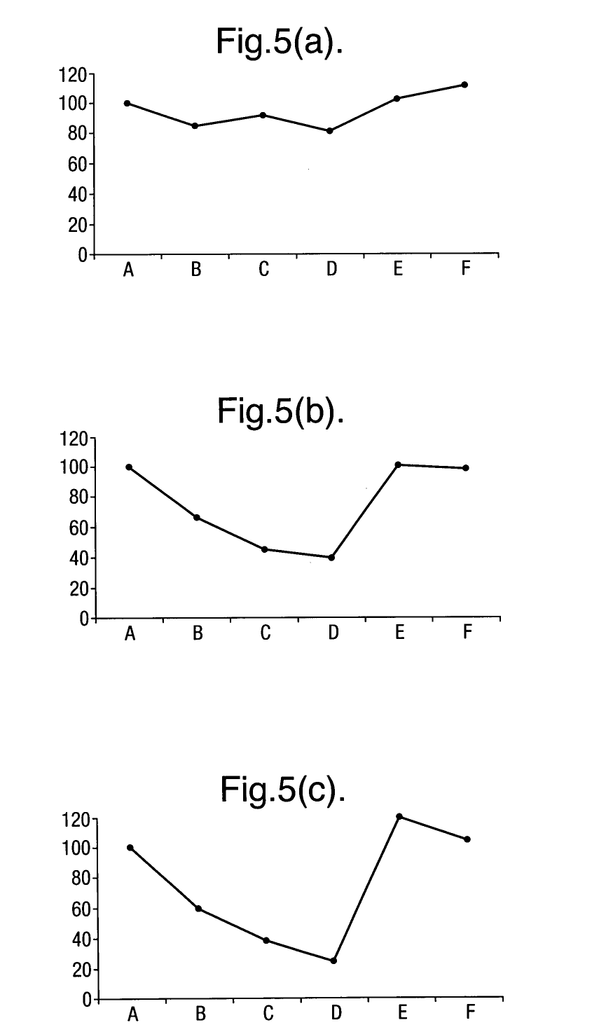

The consequences of reduced anti-ganglioside antibody binding treated cells were evaluated by measuring antibody-mediated cytotoxicity (FIGS. 4 and 5). Thus, cells from each day and NB-DNJ concentration were also assayed for anti-ganglioside antibody-mediated cytotoxicity. Lysis studies were conducted using anti-ganglioside antibodies in conjunction with fresh human serum as a source of complement. Cell viability was quantitated by colourimetric assay measuring LDH release upon cell lysis.

After coating onto 96 well tissue culture plates (5×10³ cells/well), the cells were incubated with anti-GT1b antibody at 10 μg/ml for 1 hour at room temperature. Then, 10% human serum was added as a source of complement for a further hour at 37° C. Complement-mediated cell lysis was measured using a Cytotox 96 cytotoxic assay kit (Promega), following the instructions supplied. LDH released upon cell lysis was measured by a colourimetric assay; a tetrazolium salt is converted into a red formazan product. Cell lysis at each concentration and time point was compared with that at day 0. Any lysis resulting from serum treatment alone was subtracted. Each data point in FIGS. 4 and 5 is the mean of triplicates and error bars are sem.

Only cells treated with 100 uM or 500 uM showed protection from complement mediated lysis (approximately 60% and 80% protection respectively).

These data indicate that a reduction in antibody binding of about 70% or greater is required in order to protect the cells from complement-mediated cytotoxicity in this model system.

(iii) Analytical Studies on Ganglioside Levels

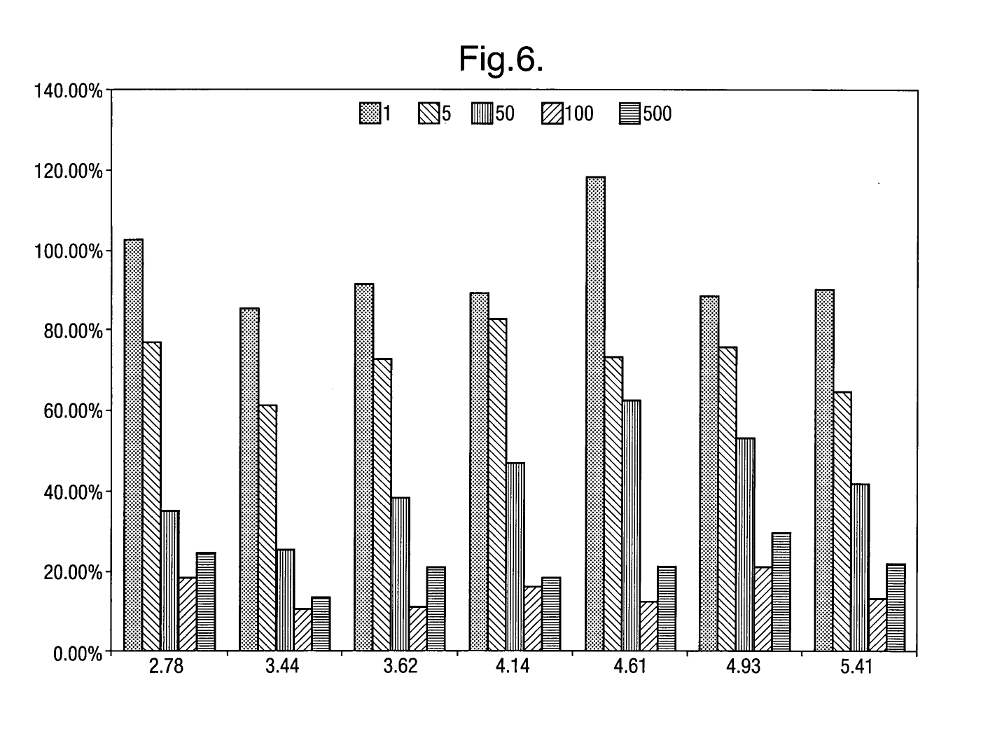

HPLC analysis of the treated and untreated cells was performed to determine the extent of GSL depletion that results from NB-DNJ treatment. The preliminary analysis on day 3 (FIG. 6) (no peaks assigned as yet, but all GSL species) shows that NB-DNJ reduced GSL levels in the PC12 cells as predicted to 10-20% of control.

Thus, data presented herein support that the targeting of antibodies to the cell membrane is dependent not only on the presence of the glycolipid antigens but also on the concentration of glycolipids in the cell membrane. In particular, the data support that reducing the glycolipid level to below a threshold level, using an inhibitor of glycolipid biosynthesis, reduces anti-glycolipid binding sufficiently enough to prevent cellular injury.

### Example 6a

### Ganglioside Specificities of Serum IgG from GBS Patients

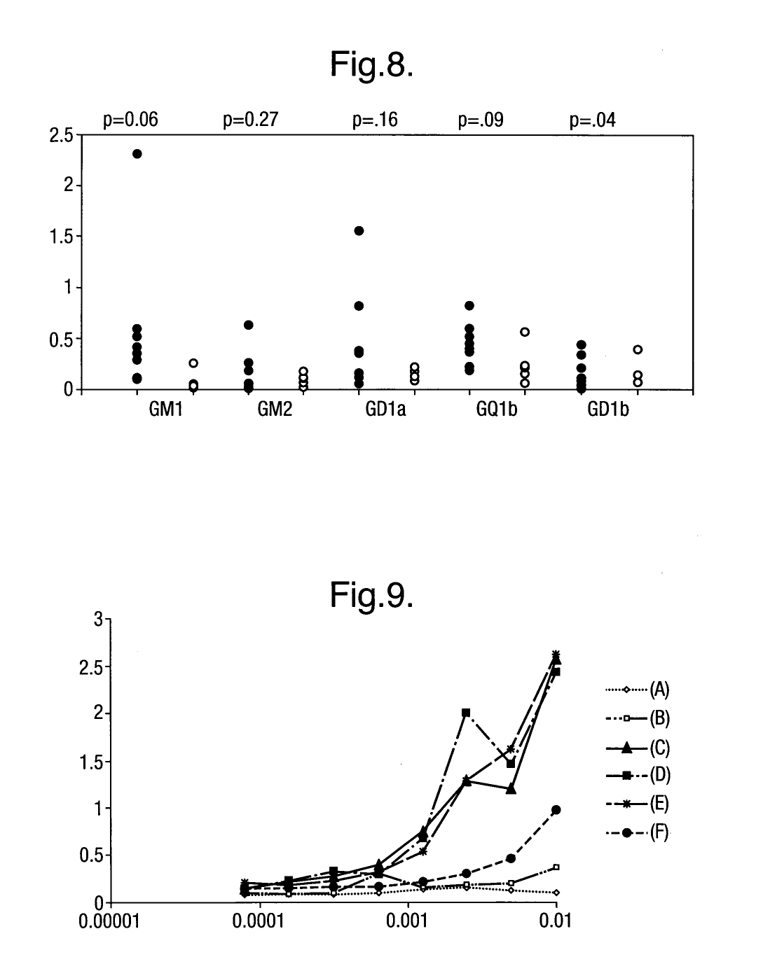

ELISA was carried out using twelve patient () and four control (∘) serum samples whose binding to five gangliosides (GM1, GM2, GD1a, GQ1b and GD1b) was assessed (FIG. 8). Antibodies reactive to all of these have been observed in some GBS patients. In general, IgG binding appeared to be higher for patient than control sera. Most significantly distinct results were obtained for IgG binding to GM1 and GQ1b, which, interestingly, are the classical epitopes for GBS and MFS respectively. The affinities of samples tested were consistent with this.

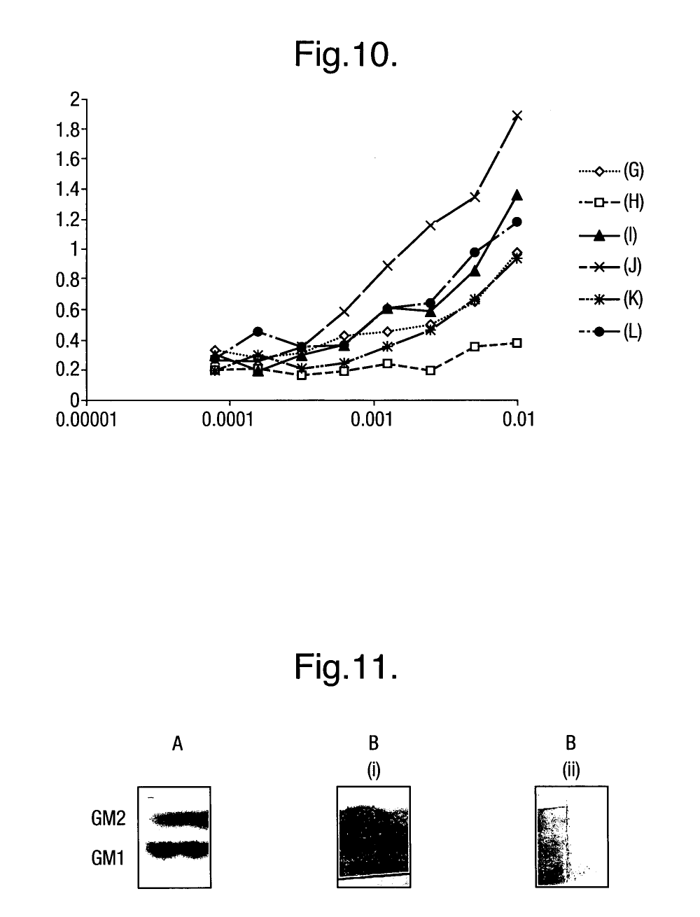

GBS patient sera binding to GM1 and GQ1b was therefore analysed further. Four patient sera samples with GM1 binding activity, and four with GQ1b binding activity, as well as control sera samples from healthy individuals were selected. Further ELISAs were then carried out, whereby increasing sera dilution factors were used to obtain binding curves (FIGS. 9 and 10).

The binding curves obtained showed a continuum in sera anti-ganglioside binding affinities, with overlap between levels patient and control binding (FIGS. 9 and 10). Implications for the non-discrete nature of human sera antigenic reactivity are discussed below (Discussion). A positive binding patient serum and a negative control serum for GM1 and GQ1b were selected for use in drug treatment experiments (described in Example 6c).

### Example 6b

### Fluorescence Microscopy of Neuroblastoma Cells using GBS Sera

Fluorescence microscopy was used to demonstrate levels of patient and control sera binding to intact Neuroblastoma NB1 cells treated with various NB-DNJ and NB-DGJ concentrations. Both control and patient sera had a wide range of reactivities to cell surface, summarised in table 6 below. A decrease in fluorescence was apparent in drug-treated cells with some GBS serum samples, such as patient 7. Some decrease in control sera binding was also observed on drug treated cells, indicating that a carbohydrate antigen may be at least partly responsible for the non-specific binding. Other samples did not show this characteristic, however. This non-specific staining hinders interpretation of results when using whole sera. Cell staining indicated the necessity for a technique which provides isolated antigen, thereby reducing the potential of non-specific staining.

TABLE 6
Cell surface serum binding to NB1 cells; both patient and control sera
displayed a large range of IgG reactivities to cell surface antigen

| | Staining |
|---|---|
| Control | |
| c1 | +++ |
| c2 | − |
| c3 | ++ |
| c4 | ++ |
| Patients | |
| p1 | + |
| p2 | + |
| p3 | + |
| p4 | − |
| p5 | − |
| p6 | + |
| p7 | ++ |
| p8 | +++ |
| p9 | + |
| p10 | +++ |
| p11 | + |

### Example 6c

### Patient and Control Sera Binding to Cellular GM1 Extract

TLC is a technique which provides isolated antigen so that sera binding to specific antigen could be detected.

Purified GM1 and GM2 were run on TLC plates, and detection was conducted by orcinol staining and immuno-overlay (FIG. 11). When the immuno-overlay was conducted using patient serum, antibody binding was observed at the same height as the orcinol stained GM1 band. When the control serum was used, no band was observed indicating a lack of anti-GM1 antibody binding. The selected patient sera thus had sufficient anti-GM1 binding affinity for detection by TLC.

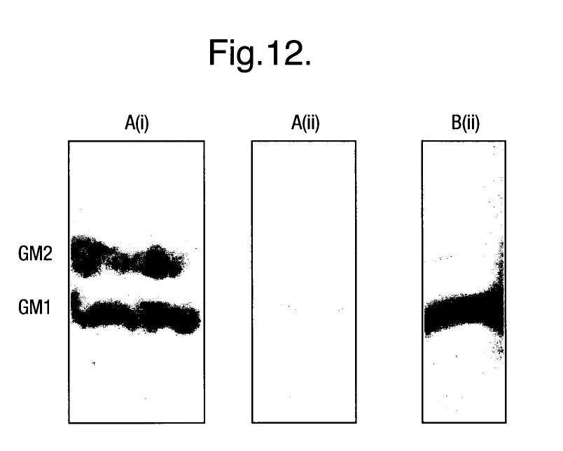

Having observed that the immuno-overlay specifically detected autoantibody reactivity from ex-vivo samples, next it was necessary to establish that levels of GM1 extracted from RAW cells were sufficient for this method. GSL was extracted from cells and separated by TLC. RAW extract was run in two lanes parallel to purified GM1 and GM2. Orcinol staining of ganglioside standards was then compared to orcinol and immuno-overlay detection of RAW extract (FIG. 12). As hoped, bands were observed at the same height as GM1 standards, showing that RAW cells contained sufficient GM1 for detection by immuno-overlay and were suitable for drug inhibition assays.

### Example 6d

### TLC Shows a GBS Sera Specific Decrease in Antibody Binding to Extract from Drug Treated Cells

GSL was extracted from RAW cells incubated in a range of NB-DNJ and NB-DGJ concentrations from 0 to 1 mM and separated by TLC. Extract was run parallel to purified GM1 on duplicate TLC plates. Immuno-overlay was carried out using patient ‘8’ and control ‘4’ sera.

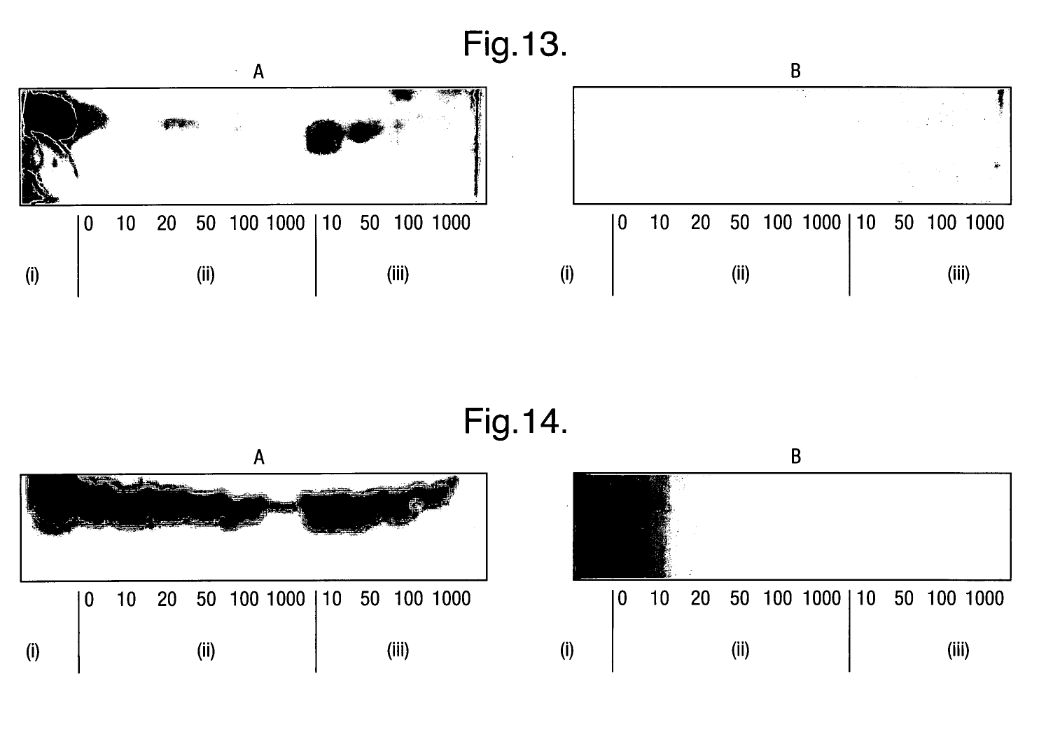

The overlay in which patient serum was used showed a drug dependent decrease in antibody binding to GM1, whereas no staining was observed with control serum (FIG. 13). The drug dependent decrease was apparently slightly more efficient with NB-DGJ than NB-DGJ despite calculated Ki values.

The procedure was then repeated with standardisation of the amount of material loaded onto the gel (FIG. 14). This was calculated using the bicinchoninic acid protein quantification method (Chan et al., 1993). Again, results showed progressively lower levels of patient anti-GM1 antibody binding as drug concentration was increased. The effect appeared to be more pronounced with NB-DGJ than NB-DNJ. No binding was observed when the overlay was conducted with control serum, indicating specificity of this effect to GBS auto-antibody.

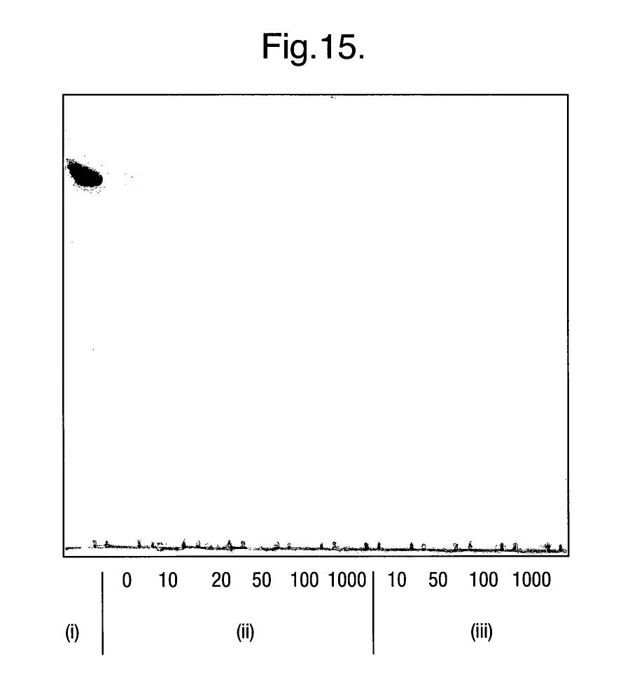

A third TLC plate (FIG. 15) with standardised amounts of sample was run in an identical manner to those in FIG. 14 but stained with orcinol. The orcinol stained plate showed a drug concentration-dependent decline in GM1 levels, concomitant with the decrease in antoantibody binding observed in FIG. 14.

The data from TLC analysis (FIGS. 13 to 15) indicates that imino-sugars can deplete GBS-specific epitope in RAW cells. To extend these findings to other cell-types, a similar strategy was adopted in a more complex tissue culture. The PC12 cell-line, characterised by neurite outgrowths, is known to contain GQ1b, which is the principle antigen of MFS auto-antibody. Patient sera were assessed for antibody anti-GQ1b reactivity through ELISAs (FIGS. 8, 10). Sera from MFS patient ‘9’, which had high anti-GQ1b reactivity was selected for use in PC12 extract TLC overlays.

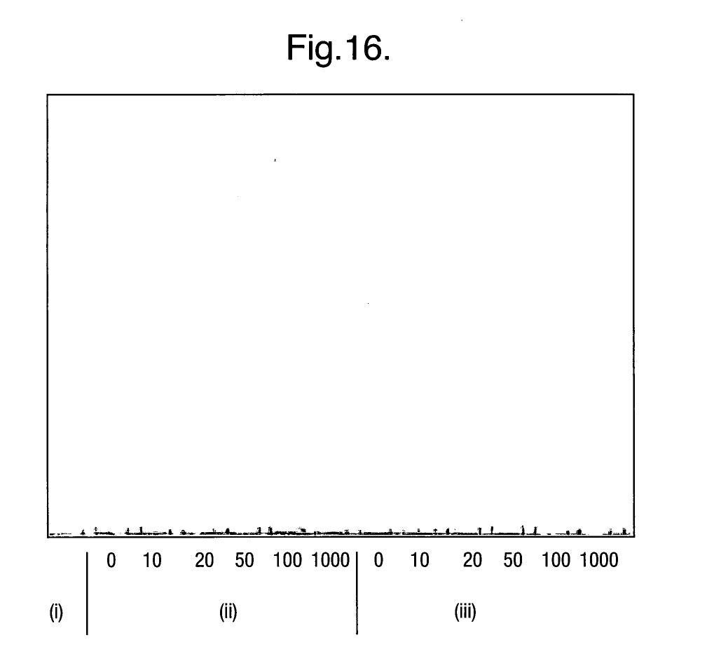

TLC was carried out on extracts from drug-treated PC12 cells. Staining with orcinol revealed an abundance of glycolipids, but lack of GQ1b (FIG. 16). TLC immuno-overlay was carried out nonetheless, but showed no antibody binding, presumably due to lack of appropriate antigen. A more sensitive analysis of PC12 ganglioside therefore was required.

### Example 6e

### HPLC Analysis of PC12 Gangliosides

In the absence of detectable binding by TLC immuno-overlay, a new technique was necessary to analyse the identity and relative abundance of gangliosides present on PC12 cells. To this end, HPLC was carried out on ganglioside extracted from PC12 cells grown in a range of NB-DGJ and NB-DNJ concentrations from 0-1 mM.

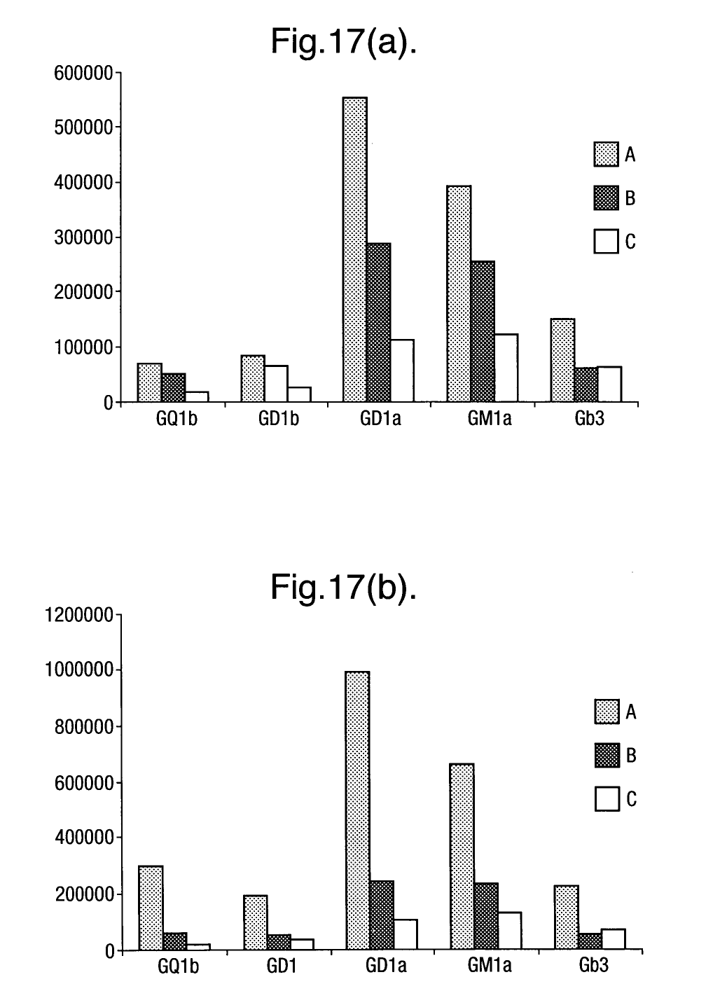

Results from HPLC (FIG. 17) revealed that GQ1b was present in PC12 cells although levels were too low for detection by TLC. Both drugs caused significant ganglioside depletion in PC12 cells at 50 μM, comparable to the apparent IC₅₀ values estimated from orcinol stained RAW TLC results (FIG. 15). Again, NB-DGJ appears to be more a potent inhibitor than NB-DNJ. Further depletion could be seen when cells were incubated with 1 mM NB-DNJ or NB-DGJ.

1.-37. (canceled)

38. A method of treating a glycolipid-mediated autoimmune disease, which method comprises administering to a patient in need of such treatment an effective amount of an inhibitor of glycolipid biosynthesis.

39. A method according to claim 38 wherein the inhibitor of glycolipid biosynthesis is an inhibitor of a glycosyltransferase or a sulfotransferase.

40. A method according to claim 39 wherein the glycosyltransferase is a glucosyltransferase, sialyltransferase, galactosyltransferasae, ceramide galactosyltransferase, fucosyltransferase, or N-acetylhexosaminetransferase.

41. A method according to claim 38 wherein the inhibitor of glycolipid biosynthesis is an inhibitor of glucosylceramide synthase.

42. A method according to claim 38 wherein the inhibitor of glycolipid biosynthesis is an inhibitor of ceramide biosynthesis.

43. A method according to claim 38 wherein the inhibitor of glycolipid biosynthesis is an inhibitor of serine palmitoyltransferase or an inhibitor of dihydroceramide synthase.

44. A method according to claim 38 wherein the inhibitor of glycolipid biosynthesis is a compound of one of the following formulae (I), (II), (III), (IV), (V), (IX) and (XII): 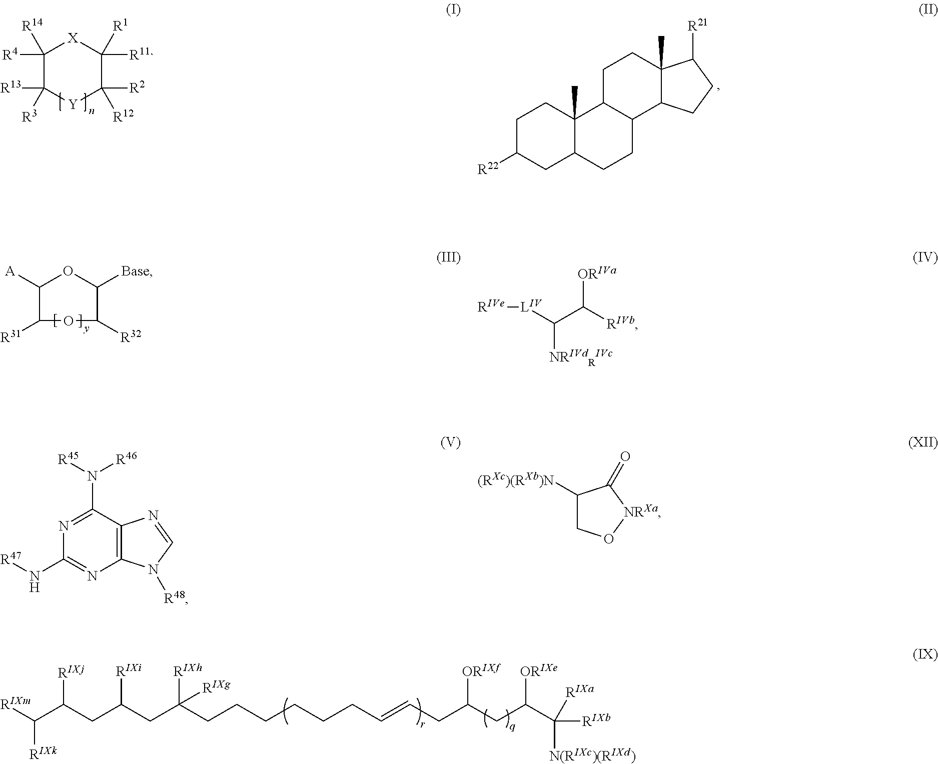
wherein:
X is O S or NR⁵;
R⁵ is hydrogen, substituted or unsubstituted C₁₋₂₀ alkyl, substituted or unsubstituted C₁₋₂₀ alkylene-aryl, substituted or unsubstituted C₁₋₂₀ alkylene-C₃₋₂₀ heteroaryl, substituted or unsubstituted C₁₋₂₀ alkylene-C₃₋₂₅ cycloalkyl, substituted or unsubstituted C₁₋₂₀ alkylene-C₃₋₂₀ heterocyclyl, substituted or unsubstituted C₁₋₂₀ alkylene-O—C₃₋₂₀ heterocyclyl, substituted or unsubstituted aryl, substituted or unsubstituted C₃₋₂₀ heteroaryl, substituted or unsubstituted C₃₋₂₅ cycloalkyl or substituted or unsubstituted C₃₋₂₀ heterocyclyl, or R⁵ forms, together with R¹, R¹¹, R⁴ or R¹⁴, a substituted or unsubstituted C₁₋₆ alkylene group, wherein said C₁₋₂₀ alkyl and C₁₋₂₀ alkylene are optionally interrupted by N(R′), O, S or arylene wherein R′ is H, C₁₋₆ alkyl or aryl;
n is 0 or 1;
Y is O, S or CR⁶R¹⁶;
R¹, R¹¹, R⁴ and R¹⁴, which may be the same or different, are independently selected from hydrogen, hydroxyl, carboxyl, amino, thiol, substituted or unsubstituted C₁₋₂₀ alkyl, substituted or unsubstituted C₁₋₂₀ alkoxy, substituted or unsubstituted aryloxy, acyl, ester, acyloxy, C₁₋₁₀ alkylamino, di(C₁₋₁₀)alkylamino, amido, acylamido, —O—C₃₋₂₅ cycloalkyl and —O—C₃₋₂₀ heterocyclyl, provided that one of R¹, R¹¹, R⁴ and R¹⁴ may form, together with R⁵, a substituted or unsubstituted C₁₋₆ alkylene group, wherein said C₁₋₂₀ alkyl is optionally interrupted by N(R′), O, S or arylene;
R², R¹², R³, R¹³, R⁶ and R¹⁶, which may be the same or different, are independently selected from hydrogen, hydroxyl, carboxyl, amino, thiol, substituted or unsubstituted C₁₋₂₀ alkyl, substituted or unsubstituted C₁₋₂₀ alkoxy, substituted or unsubstituted aryloxy, acyl, ester, acyloxy, C₁₋₁₀ alkylamino, di(C₁₋₁₀)alkylamino, amido, acylamido —O—CO₃₋₂₅ cycloalkyl and —O—C₃₋₂₀ heterocyclyl, wherein said C₁₋₂₀ alkyl is optionally interrupted by N(R′), O, S or arylene;
R²¹ is selected from oxo, -L³⁰-R²³, -L³⁰-C(O)N(H)—R²⁴ and a group of the following formula (VI): 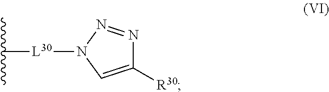
L³⁰ is substituted or unsubstituted C₁₋₂₀ alkylene which is optionally interrupted by N(R′), O, S or arylene;
R²³ is carboxyl, hydroxyl, ester, phosphonate ester, phosphate ester, phosphoric acid and phosphonic acid;
R²⁴ is C₁₋₂₀ alkyl which is unsubstituted or substituted with one or more groups selected from carboxyl, hydroxyl, ester, phosphonate ester, phosphate ester, phosphoric acid and phosphonic acid, wherein said C₁₋₂₀ alkyl is optionally interrupted by N(R′), O, S or arylene;
R³⁰ is C₁₋₂₀ alkyl which is unsubstituted or substituted with one or more groups selected from carboxyl, hydroxyl, ester, amino, phosphonate ester, phosphate ester, phosphoric acid and phosphonic acid, wherein said C₁₋₂₀ alkyl is optionally interrupted by N(R′), O, S or arylene, and
R²² is hydroxyl, oxo, acyloxy, phosphoric acid or —OC(O)-alk-C(O)OH, wherein alk is substituted or unsubstituted C₁₋₂₀ alkylene which is optionally interrupted by N(R′), O, S or arylene;
Base is selected from a group of any one of the following formulae (a), (b), (c), (d), (e), (f) and (g): 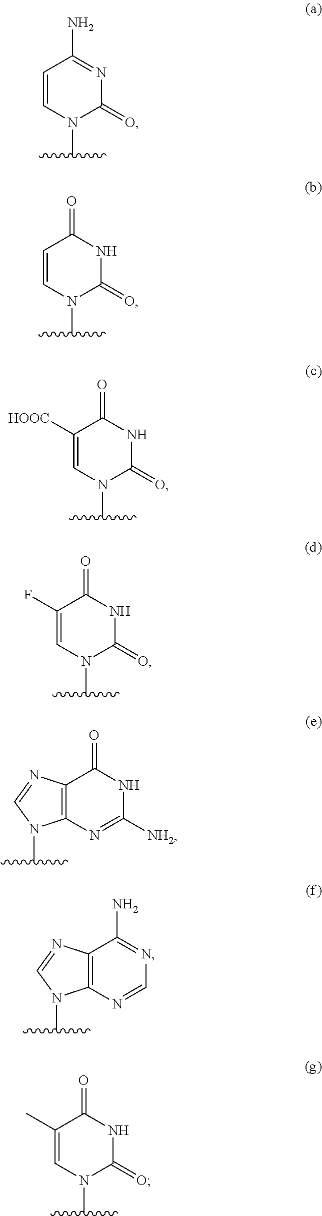
y is 0 or 1;
R³¹ is OH; R³² is H or OH; or, provided that y is O, R³¹ and R³² together form —O—C(R³³)(R³⁴)—O—, wherein R³³ and R³⁴ are independently selected from H and methyl;
A is substituted or unsubstituted C₁₋₂₀ alkyl, substituted or unsubstituted C₁₋₂₀ alkylene-aryl, substituted or unsubstituted C₁₋₂₀ alkylene-C₃₋₂₀ heteroaryl, substituted or unsubstituted C₁₋₂₀ alkylene-C₃₋₂₅ cycloalkyl or substituted or unsubstituted C1120 alkylene-C₃₋₂₀ heterocyclyl, wherein said C₁₋₂₀ alkyl and C₁₋₂₀ alkylene are optionally interrupted by N(R′), O, S or arylene, wherein R′ is H, C₁₋₆ alkyl or aryl, or A is a group of any one of the following formulae (g) to (k): 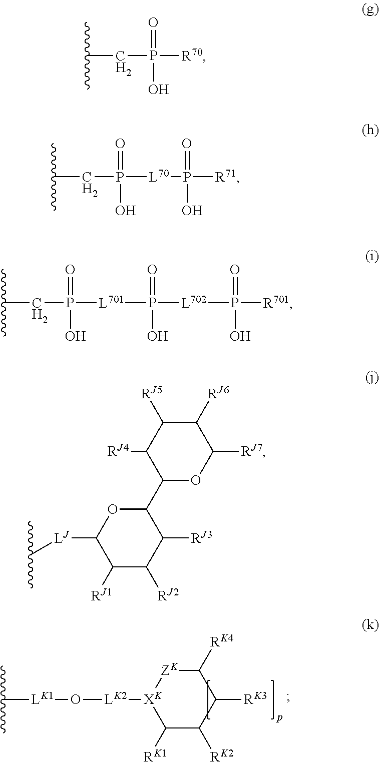
L⁷⁰, L⁷⁰¹ and L⁷⁰² are independently selected from —O—, —C(R³⁵)(R³⁶)— and —NH—, wherein R³⁵ and R³⁶ are independently selected from H, OH and CH₃;
R⁷⁰, R⁷¹ and R⁷⁰¹ are selected from OH, substituted or unsubstituted C₁₋₂₀ alkyl, substituted or unsubstituted C₁₋₂₀ alkoxy, substituted or unsubstituted C₁₋₁₀ alkylamino and -L⁷¹-(X²)ₘ-L⁷²-R⁷²; wherein m is 0 or 1; X² is O, S, —C(R⁴⁵)(R⁴⁶)— or —O—C(R⁴⁵)(R⁴⁶)—, wherein R⁴⁵ and R⁴⁶ are independently selected from H, OH, phosphonic acid or a phosphonic acid salt; L⁷¹ and L⁷² are independently selected from a single bond and substituted or unsubstituted C₁₋₂₀ alkylene, which C₁₋₂₀ alkylene is optionally interrupted by N(R′), O, S or arylene, wherein R′ is H, C₁₋₆ alkyl or aryl; and R⁷² is CO₃₋₂₅ cycloalkyl or C₃₋₂₀ heterocyclyl;
L^{J} is substituted or unsubstituted C₁₋₂₀ alkylene;
R^{J1}, R^{J2}, R^{J3}, R^{J4}, R^{J5}, R^{J6} and R^{J7}, which are the same or different, are independently selected from hydrogen, hydroxyl, carboxyl, amino, thiol, substituted or unsubstituted C₁₋₂₀ alkyl, substituted or unsubstituted C₁₋₂₀ alkoxy, substituted or unsubstituted aryloxy, acyl, ester, acyloxy, C₁₋₁₀ alkylamino, di(C₁₋₁₀)alkylamino, amido, acylamido, —N(H)C(O)CH═CH—R^{J8}, —C₃₋₂₅ cycloalkyl and —O—C₃₋₂₀ heterocyclyl, wherein said C₁₋₂₀ alkyl is optionally interrupted by N(R′), O, S or arylene, and wherein R^{J8} is substituted or unsubstituted C₁₋₂₀ alkyl;
L^{K1} and L^{K2}, which are the same or different, are independently selected from a single bond and substituted or unsubstituted C₁₋₂₀ alkylene;
X^{K} is N or C(R^{K6}), wherein R^{K6} is H, COOH or ester;
Z^{K} is O or CH(R^{K5});
p is 0 or 1;
R^{K1}, R^{K2}, R^{K3}, R^{K4} and R^{K5}, which are the same or different, are independently selected from hydrogen, hydroxyl, carboxyl, amino, thiol, substituted or unsubstituted C₁₋₂₀ alkyl, substituted or unsubstituted C₁₋₂₀ alkoxy, substituted or unsubstituted aryloxy, acyl, ester, acyloxy, C₁₋₁₀ alkylamino, di(C₁₋₁₀)alkylamino, amido, acylamido, —O—C₃₋₂₅ cycloalkyl and —O—C₃₋₂₀ heterocyclyl, wherein said C₁₋₂₀ alkyl is optionally interrupted by N(R′), O, S or arylene;
R^{IVa} and R^{IVd}, which are the same or different, are independently selected from H,
substituted or unsubstituted C₁₋₆ alkyl or substituted or unsubstituted phenyl;
R^{IVb} is H, substituted or unsubstituted aryl, —CH═CHR^{IVf}, or substituted or unsubstituted C₁₋₂₀ alkyl, which C₁₋₂₀ alkyl is optionally interrupted by N(R′), O, S or arylene wherein R′ is H, C₁₋₆ alkyl or aryl;
R^{IVc} is H, substituted or unsubstituted C₁₋₆ alkyl, substituted or unsubstituted phenyl or —C(O)R^{IVg};
R^{IVf} is H or substituted or unsubstituted C₁₋₂₀ alkyl, which C₁₋₂₀ alkyl is optionally interrupted by N(R′), O, S or arylene;
R^{IVg} is H or substituted or unsubstituted C₁₋₂₀ alkyl, which C₁₋₂₀ alkyl is optionally interrupted by N(R′), O, S or arylene;
R^{IVe} is H, hydroxyl, carboxyl, amino, thiol, substituted or unsubstituted C₁₋₂₀ alkyl, substituted or unsubstituted C₁₋₂₀ alkoxy, substituted or unsubstituted aryloxy, acyl, ester, acyloxy, C₁₋₁₀ alkylamino, di(C₁₋₁₀)alkylamino, amido, acylamido, —O—C₃₋₂₅ cycloalkyl, —O—C₃₋₂₀ heterocyclyl, substituted or unsubstituted aryl, substituted or unsubstituted C₃₋₂₀ heteroaryl, substituted or unsubstituted C₃₋₂₅ cycloalkyl or substituted or unsubstituted C₃₋₂₀ heterocyclyl, which C₁₋₂₀ alkyl is optionally interrupted by N(R′), O, S or arylene;
L^{IV} is substituted or unsubstituted C₁₋₂₀ alkylene which C₁₋₂₀ alkylene is optionally interrupted by N(R′), O, S or arylene;
R⁹¹ and R⁹² which are the same or different, are independently selected from H, substituted or unsubstituted C₁₋₂₀ alkyl, substituted or unsubstituted aryl and -L⁹¹-R⁹⁵, wherein L⁹¹ is substituted or unsubstituted C₁₋₂₀ alkylene, wherein said C₁₋₂₀ alkyl and said C₁₋₂₀ alkylene are optionally interrupted by N(R′), O, S or arylene wherein R′ is H, C₁₋₆ alkyl or aryl, and wherein R⁹⁵ is substituted or unsubstituted aryl, amino, C₁₋₁₀ alkylamino or di(C₁₋₁₀)alkylamino;
R⁹³ is -L⁹²-R⁹⁶, wherein L⁹² is a single bond or substituted or unsubstituted C₁₋₂₀ alkylene, which C₁₋₂₀ alkylene is optionally interrupted by N(R′), O, S or arylene, and wherein R⁹⁶ is amido or substituted or unsubstituted aryl;
R⁹⁴ is H or substituted or unsubstituted C₁₋₂₀ alkyl, which C₁₋₂₀ alkyl is optionally interrupted by N(R′), O, S or arylene;
q is 0 or 1;
r is 0 or 1;
R^{IXa} is H, COOH or an unsubstituted or substituted ester;
R^{IXb} is an unsubstituted or substituted C₁₋₆ alkyl;
R^{IXc} and R^{IXd}, which are the same or different, are each independently selected from H, unsubstituted or substituted C₁₋₆ alkyl and unsubstituted or substituted phenyl;
R^{IXe} and R^{IXf}, which are the same or different, are each independently selected from H, unsubstituted or substituted C₁₋₆ alkyl, unsubstituted or substituted phenyl and unsubstituted or substituted acyl;
either (a) one of R^{IXg} and R^{IXh} is H and the other is OR^{Ixr}, wherein R^{IXr} is selected from H, unsubstituted or substituted C₁₋₆ alkyl, unsubstituted or substituted phenyl and unsubstituted or substituted acyl, or (b) R^{IXg} and R^{IXh} together form an oxo group;
R^{IXi} is H, unsubstituted or substituted C₁₋₆ alkyl, unsubstituted or substituted C₁₋₆ alkoxy and unsubstituted or substituted phenyl;
R^{IXj} is H, unsubstituted or substituted C₁₋₆ alkyl or a group of the following formula (X): 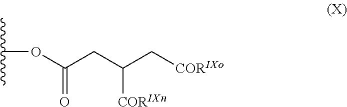
in which R^{IXn} and R^{IXo}, which are the same or different, are each independently selected from OH, unsubstituted or substituted C₁₋₆ alkoxy, unsubstituted or substituted phenoxy, amino, unsubstituted or substituted C₁₋₆ alkylamino and unsubstituted or substituted di(C₁₋₆)alkylamino;
R^{IXk} is H, unsubstituted or substituted C₁₋₆ alkyl or a group of the following formula (XI): 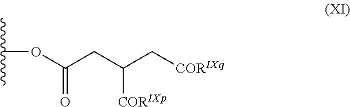
in which R^{IXp} and R^{IXq}, which are the same or different, are each independently selected from OH, unsubstituted or substituted C₁₋₆ alkoxy, unsubstituted or substituted phenoxy, amino, unsubstituted or substituted C₁₋₆ alkylamino and unsubstituted or substituted di(C₁₋₆)alkylamino;
R^{IXm} is selected from H and unsubstituted or substituted C₁₋₂₀ alkyl, which C₁₋₂₀ alkyl is optionally interrupted by N(R′), O, S or phenylene, wherein R′ is H, C₁₋₆ alkyl or phenyl;
R^{Xa} is H, substituted or unsubstituted C₁₋₂₀ alkyl, substituted or unsubstituted C₁₋₂₀ alkylene-aryl, substituted or unsubstituted C₁₋₂₀ alkylene-C₃₋₂₀ heteroaryl, substituted or unsubstituted C₁₋₂₀ alkylene-C₃₋₂₅ cycloalkyl, substituted or unsubstituted C₁₋₂₀ alkylene-C₃₋₂₀ heterocyclyl, substituted or unsubstituted C₁₋₂₀ alkylene-O—C₃₋₂₀ heterocyclyl, substituted or unsubstituted aryl, substituted or unsubstituted C₃₋₂₀ heteroaryl, substituted or unsubstituted C₃₋₂₅ cycloalkyl or substituted or unsubstituted C₃₋₂₀ heterocyclyl wherein said C₁₋₂₀ alkyl and C₁₋₂₀ alkylene are optionally interrupted by N(R′), O, S or arylene wherein R′ is H, C₁₋₆ alkyl or aryl; and
R^{Xb} and R^{Xc}, which are the same or different, are independently selected from H, unsubstituted or substituted C₁₋₁₀ alkyl and unsubstituted or substituted aryl;
or a pharmaceutically acceptable salt thereof.

45. A method according to claim 44 wherein the compound has the following formula (Ia): 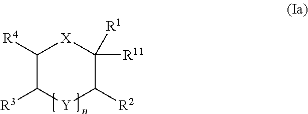
wherein Y is O, S or CHR⁶; and X, n, R¹, R², R³, R⁴, R⁵, R⁶ and R¹¹ are as defined in claim 7.

46. A method according to claim 45 wherein X is NR⁵; n is 1; Y is CHR⁶; R¹¹ is H; and R⁵ is selected from:
hydrogen;
unsubstituted or substituted C₁₋₂₀ alkyl which is optionally interrupted by O; and —C₁₋₄ alkylene-O—C₃₋₂₀ heterocyclyl, wherein said C₁₋₄ alkylene is unsubstituted and said C₃₋₂₀ heterocyclyl is a group of the following formula (m): 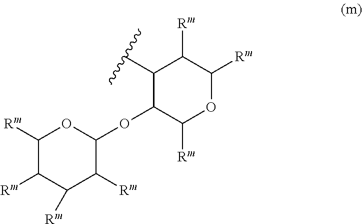
in which each R^{m} is independently selected from C₁₋₆ alkyl, OH, acyloxy, SH, C₁₋₆ alkoxy, aryloxy, amino, C₁₋₁₀ alkylamino, di(C₁₋₁₀)alkylamino, amido and acylamido;
or R⁵ forms, together with R⁴, a substituted or unsubstituted C₁₋₆ alkylene group.

47. A method according to claim 38 wherein the inhibitor of glycolipid biosynthesis is selected from N-butyldeoxynojirimycin; N-nonyldeoxynojirimycin; N-butyldeoxygalactonojirimycin; N-5-adamantane-1-yl-methoxypentyl-deoxynojirimycin; alpha-homogalactonojirimycin; nojirimycin; deoxynojirinycin; N7-oxadecyl-deoxynojirimycin; deoxygalactonojirimycin; N-butyl-deoxygalactonojirimycin; N-nonyl-deoxygalactonojirimycin; N-nonyl-6deoxygalactonojirimycin; N7-oxanonyl-6deoxy-DGJ; alpha-homoallonojirimycin; beta-1-C-butyl-deoxygalactonojirimycin; 1,5-dideoxy-1,5-imino-D-glucitol, 1,5-(Butylimino)-1,5-dideoxy-D-glucitol; 1,5-(Methylimino)-1,5-dideoxy-D-glucitol; 1,5-(Hexylimino)-1,5-dideoxy-D-glucitol; 1,5-(Nonylylimino)-1,5-dideoxy-D-glucitol; 1,5-(2-Ethylbutylimino)-1,5-dideoxy-D-glucitol; 1,5-(2-Methylpentylimino)-1,5-dideoxy-D-glucitol; 1,5-(Benzyloxycarbonylimino)-1,5-dideoxy-D-glucitol, tetraacetate; 1,5-(Phenylacetylimino)-1,5-dideoxy-D-glucitol, tetraacetate; 1,5-(Benzoylimino)-1,5-dideoxy-D-glucitol, tetraacetate; 1,5-(Butylimino)-1,5-dideoxy-D-glucitol, tetraacetate; 1,5-(Ethyl malonylimino)-1,5-dideoxy-D-glucitol, tetraacetate; 1,5-(Hexylimino)-1,5-dideoxy-D-glucitol, tetraacetate; 1,5-(Nonylimino)-1,5-dideoxy-D-glucitol, tetraacetate; 1,5-(Benzyloxycarbonylimino)-1,5-dideoxy-D-glucitol, tetraisobutyrate; 1,5-(Butylimino)-1,5-dideoxy-D-glucitol, tetrabutyrate; 1,5-(Butylimino)-1,5-dideoxy-D-glucitol, tetrapropionate; 1,5-(Butylimino)-1,5-dideoxy-D-glucitol, tetrabenzoate; 1,5-Dideoxy-1,5-imino-D-glucitol, tetraisobutyrate; 1,5-(Hydrocinnamoylimino)-1,5-dideoxy-D-glucitol, tetraacetate; 1,5-(Methyl malonylimino)-1,5-dideoxy-D-glucitol, tetraacetate; 1,5-(Butylimino)-1,5-dideoxy-D-glucitol, tetraisobutyrate; 1,5-(Butylimino)-1,5-dideoxy-4R,6-O-(phenylmethylene)-D-glucitol, diacetate; 1,5-[(Phenoxymethyl)carbonylimino]-1,5-dideoxy-D-glucitol, tetraacetate; 1,5-[(Ethylbutyl)imino]-1,5-dideoxy-D-glucitol, tetraacetate; 1,5-(Butylimino)-1,5-dideoxy-D-glucitol, 2,3-diacetate; 1,5-(Hexylimino)-1,5-dideoxy-4R,6-O-(phenylmethylene)-D-glucitol, diacetate; 1,5-(Hexylimino)-1,5-dideoxy-D-glucitol, 2,3-diacetate; 1,5-[(2-Methylpentyl)imino]-1,5-dideoxy-D-glucitol, tetraacetate; 1,5-(Butylimino)-1,5-dideoxy-D-glucitol, 6-acetate; 1,5-[(3-Nicotinoyl)imino]-1,5-dideoxy-D-glucitol, tetraacetate; 1,5-(Cinnamoylimino)-1,5-dideoxy-D-glucitol, tetraacetate; 1,5-(Butylimino)-1,5-dideoxy-D-glucitol, 2,3-dibutyrate; 1,5-(Butylimino)-1,5-dideoxy-4R,6-O-(phenylmethylene)-g-glucitol, 2,3-dibutyrate; 1,5-(Phenylacetylimino)-1,5-dideoxy-D-glucitol, tetraisobutyrate; 1,5-[(4-Chlorophenyl)acetylimino]-1,5-dideoxy-D-glucitol, tetraacetate; 1,5-[(4-Biphenyl)acetylimino]-1,5-dideoxy-D-glucitol, tetraacetate; 1,5-(Benzyloxycarbonylimino)-1,5-dideoxy-D-glucitol, tetrabutyrate; 1,5-Dideoxy-1,5-imino-D-glucitol, tetrabutyrate; 3,4,5-piperidinetriol, 1-propyl-2-(hydroxymethyl)-, (2S,3R,4R,5S); 3,4,5-piperidinetriol, 1-pentyl-2-(hydroxymethyl)-, (2S,3R,4R,55); 3,4,5-piperidinetriol, 1-heptyl-2-(hydroxymethyl)-, (2S,3R,4R,5S); 3,4,5-piperidinetriol, 1-butyl-2-(hydroxymethyl)-, (2S,3S,4R, SS); 3,4,5-piperidinetriol, 1-nonyl-2-(hydroxymethyl)-, (2S,3R,4R,5S); 3,4,5-piperidinetriol, 1-(1-ethyl)propyl-2-(hydroxymethyl)-, (2S,3R,4R,5S); 3,4,5-piperidinetriol, 1-(3-methyl)butyl-2-(hydroxymethyl)-, (2S,3R,4R,5S), 3,4,5-piperidinetriol, 1-(2-phenyl)ethyl-2-(hydroxymethyl)-, (2S,3R,4R,5S); 3,4,5-piperidinetriol, 1-(3-phenyl)propyl-2-(hydroxymethyl)-, (2S,3R,4R,5S); 3,4,5-piperidinetriol, 1-(1-ethyl)hexyl-2-(hydroxymethyl)-, (2S,3R,4R,5S); 3,4,5-piperidinetriol, 1-(2-ethyl)butyl-2-(hydroxymethyl)-, (2S,3R,4R,5S); 3,4,5-piperidinetriol, 1-[(2R)-(2-methyl-2-phenyl)ethyl]-2-(hydroxymethyl)-, (2S,3R,4R,5S); 3,4,5-piperidinetriol, 1-[(2S-(2-methyl-2-phenyl)ethyl]-2-(hydroxymethyl)-, (2S,3R,4R,5S), β-L-homofuconojirimycin; propyl 2-acetamido-2-deoxy-4-O-(β-D-galactopyranosyl)-3-O-(2-(N-(β-L-homofuconojirimycinyl))ethyl)-α-D-glucopyranoside; ido-N-(5-adamantane-1-yl-methoxy-pentyl)deoxynojirimycin; N-(adamantane-1-yl-methoxypentyl)-L-ido-deoxynojirimycin; N-(adamantane-1-yl-methoxypentyl)-D-galacto-deoxynojirimycin; C1-beta-(adamantane-1-yl-methoxypentyl)-deoxynojirimycin; N-methyl-C1-beta-(adamantane-1-yl-methoxypentyl)-deoxynojirimycin; N-butyl-C1-beta-(adamantane-1-yl-methoxypentyl)-deoxynojirimycin; 2-O-(adamantane-1-yl-methoxypentyl)-deoxynojirimycin; N-methyl-2-O-(adamantane-1-yl-methoxy-pentyl)-deoxynojirimycin; N-butyl-2-O-(adamantane-1-yl-methoxy-pentyl)-deoxynojirimycin; N-benzyloxycarbonyl-2-O-(adamantane-1-yl-methoxypentyl)-3,4,6-tri-O-benzyl-deoxy-nojirimycin; and N-(5-adamantane-1-yl-methoxy-pentyl)deoxynojirimycin.

48. A method according to claim 45 wherein:
X is NR⁵; Y is O or S; n is either 0 or 1;
R¹¹ is H; and
R⁵ is selected from hydrogen and a group of the following formula (VIII): 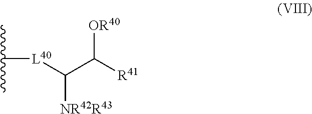
in which:
R⁴⁰ and R⁴², which are the same or different, are independently selected from H, substituted or unsubstituted C₁₋₆ alkyl or substituted or unsubstituted phenyl;
R⁴¹ is H, substituted or unsubstituted aryl, —CH═CHR⁴⁴, or substituted or unsubstituted C₁₋₂₀ alkyl, which C₁₋₂₀ alkyl is optionally interrupted by N(R′), O, S or arylene wherein R′ is H, C₁₋₆ alkyl or aryl;
R⁴³ is H, substituted or unsubstituted C₁₋₆ alkyl, substituted or unsubstituted phenyl or —C(O)R⁴⁷;
R⁴⁴ is H or substituted or unsubstituted C₁₋₂₀ alkyl, which C₁₋₂₀ alkyl is optionally interrupted by N(R′), O, S or arylene;
R⁴⁷ is substituted or unsubstituted C₁₋₂₀ alkyl, which C₁₋₂₀ alkyl is optionally interrupted by N(R′), O, S or arylene; and
L⁴⁰ is substituted or unsubstituted C₁₋₁₀ alkylene.

49. A method according to claim 45 wherein the inhibitor of glycolipid biosynthesis is selected from: D,L-threo-1-phenyl-2-decanoylamino-3-morpholino-1-propanol; D,L-threo-1-phenyl-2-hexadecanoylamino-3-morpholino-1-propanol; D-threo-1-phenyl-2-palmitoilamino-3-pyrrolidino-1-propanol; 4′-hydroxy-D-threo-1-phenyl-2-palmitoilamino-3-pyrrolidino-1-propanol; 3′,4′-ethylenedioxy-P4 and 2,5-dihydroxymethyl-3,4-dihydroxypyrrolidine.

50. A method according to claim 45 wherein:
X is O or S; n is 1; Y is CHR⁶; R¹¹ is H;
R⁶ is H, hydroxyl, acyloxy, C₁₋₂₀ alkoxy, C₁₋₁₀ alkylamino or di(C₁₋₁₀)alkylamino;
R² and R³, which may be the same or different, are independently selected from H, hydroxyl, C₁₋₂₀ alkoxy, acyloxy or acylamido;
R⁴ is H, hydroxyl, acyloxy, thiol or C₁₋₂₀ alkyl, which C₁₋₂₀ alkyl is unsubstituted or substituted with one, two, three or four groups selected from hydroxyl, acyloxy and thiol and
R¹ is C₁₋₂₀ alkoxy, aryloxy or —O—C₃₋₂₀ heterocyclyl.

51. A method according to claim 45 wherein the inhibitor of glycolipid biosynthesis is selected from:

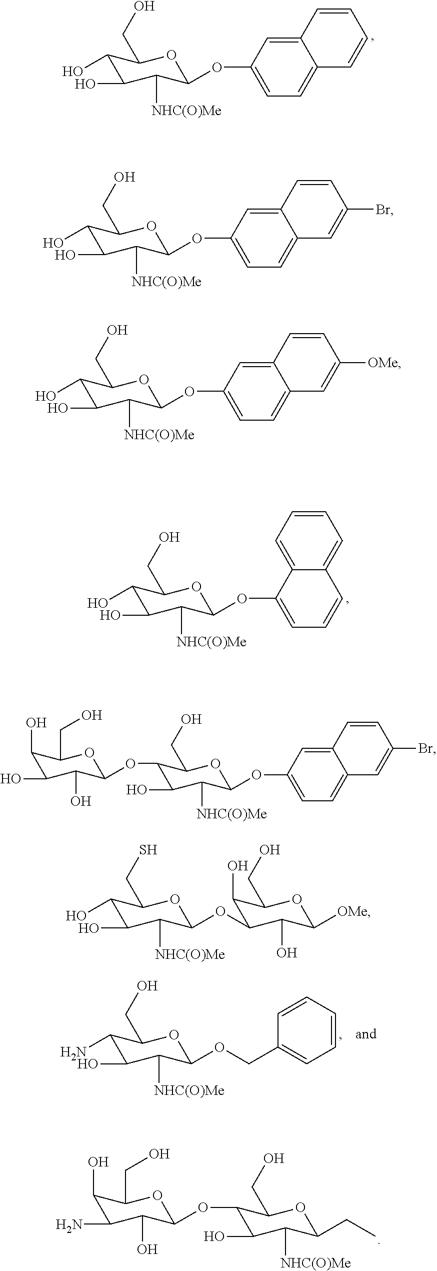

52. A method according to claim 45 wherein:
X is or S; n is 1; Y is CHR⁶;
R⁶ is H, hydroxyl, acyloxy or C₁₋₂₀ alkoxy;
R¹ and R¹¹ which may be the same or different, are independently selected from H, C₁₋₂₀ alkyl, hydroxyl, acyloxy, C₁₋₂₀alkoxy, carboxyl, ester, —O—CO₃₋₂₅ cycloalkyl, and a group of the following formula (VII): 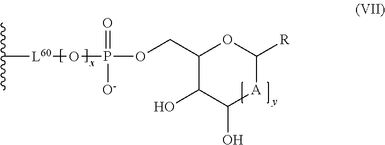
wherein L⁶⁰ is substituted or unsubstituted C₁₋₂₀ alkylene; x is 0 or 1; y is 0 or 1; A is CHR′″ and R is H, C₁₋₂₀ alkyl, C₃₋₂₀ heterocyclyl, C₃₋₂₅ cycloalkyl, aryl or C₁₋₂₀ alkoxy, wherein R′″ is hydroxyl, C₁₋₆ alkoxy, aryloxy or acyl;
R² is H, C₁₋₂₀ alkyl, hydroxyl, acyloxy or —O—C₃₋₂₀ heterocyclyl;
R³ is H, hydroxyl, acyloxy, C₁₋₂₀ alkoxy or acylamido; and
R⁴ is H, carboxyl, ester or C₁₋₂₀ alkyl which is unsubstituted or substituted with one, two, three or four groups selected from hydroxyl and thiol.

53. A method according to claim 45 wherein the inhibitor of glycolipid biosynthesis is cytidin-5′-yl sialylethylphosphonate, sialic acid or Soyasaponin I.

54. A method according to claim 44 wherein the compound is of formula (II), and wherein:
R²¹ is selected from oxo, -L³⁰-R²³, -L³⁰-C(O)N(H)—R²⁴ and a group of the following formula (VI): 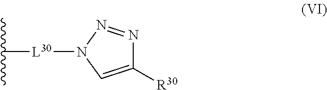
wherein
L³⁰ is substituted or unsubstituted C₁₋₆ alkylene,
R²³ is hydroxyl, carboxyl, ester or phosphate ester,
R²⁴ is C₁₋₆ alkyl which is unsubstituted or substituted with one or two carboxyl groups;
R³⁰ is C₁₋₆ alkyl which is unsubstituted or substituted with one or two groups selected from hydroxyl, carboxyl, amino and phosphonate ester; and
R²² is as defined in claim 7.

55. A method according to claim 44 wherein R²¹ is a group selected from oxo and the groups having the following structures:

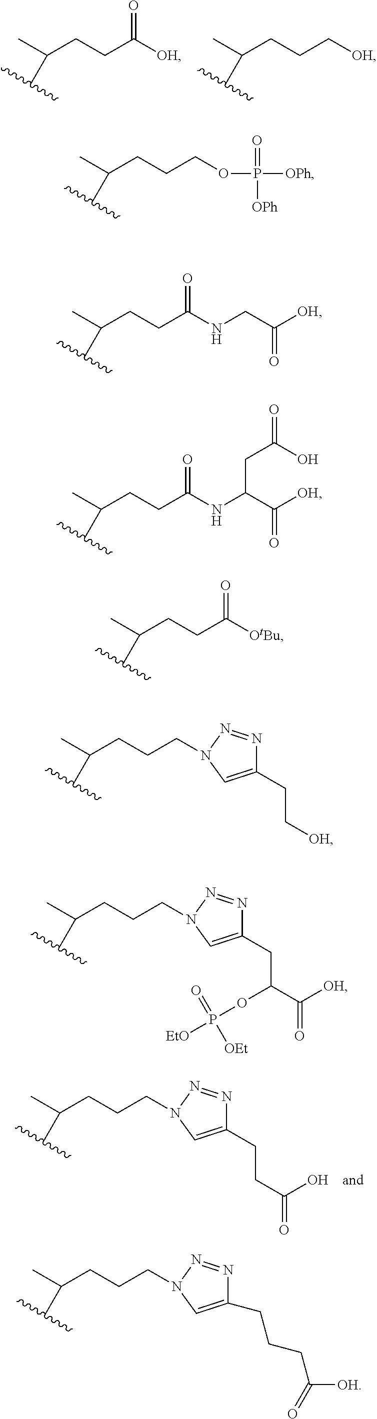

56. A method according to claim 44 wherein R²² is selected from hydroxyl, oxo, phosphoric acid, —OC(O)—CH₂—CH₂—C(O)OH and —OC(O)—CH(NH₂)—CH₂—C(O)OH.

57. A method according to claim 44 wherein the inhibitor of glycolipid biosynthesis is selected from the compounds of formula (II) listed in Table 1.

58. A method according to claim 44 wherein the compound is of formula (III) and wherein:
Base is selected from (a), (b), (c), (d) and (e);
y is 0 and R³¹ and R³² are both —OH;
A is either (g), (h) or (i);
L⁷⁰, L⁷⁰¹ and L⁷⁰² are selected from O, CH₂, CHOH, C(OH)(CH₃) and NH; and
R⁷⁰, R⁷¹ and R⁷⁰¹ are as defined in claim 7.

59. A method according to claim 44 wherein the inhibitor of glycolipid biosynthesis is selected from the compounds of formula (II) listed in Table 2.

60. A method according to claim 44 wherein the compound is of formula (IV) and wherein:
R^{IVa} and R^{IVd} are both H;
R^{IVc} is —C(O)R^{IVg};
R^{IVg} is unsubstituted C₁₋₂₀ alkyl;
R^{IVb} is —CH═CHR^{IVf}, wherein R^{IVf} is unsubstituted C₁₋₂₀ alkyl, or R^{IVb} is a group of the following formula (IVa): 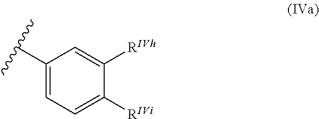
in which R^{IVh} is H, C₁₋₆ alkyl or phenyl, or R^{IVh} forms, together with R^{IVi}, a bidentate group of the structure —O-alk-O—; and R^{IVi} is H, CO₁₋₆ alkyl or phenyl, or R^{IVi} forms, together with R^{IVh}, a bidentate group of the structure —O-alk-O—, wherein alk is substituted or unsubstituted C₁₋₆ alkylene; and
R^{IVe} is OH, substituted or unsubstituted aryl, substituted or unsubstituted C₃₋₂₀ heteroaryl, substituted or unsubstituted C₃₋₂₅ cycloalkyl or substituted or unsubstituted C₃₋₂₀ heterocyclyl.

61. A method according to claim 44 wherein the compound is of formula (V) and wherein
R⁹¹ is H, —C₁₋₄ alkylene-amino, —C₁₋₄ alkylene-C₁₋₁₀ alkylamino or —C₁₋₄ alkylene-di(C₁₋₁₀)alkylamino;
R⁹² is —C₁₋₄ alkylene-phenyl, wherein said phenyl is substituted or unsubstituted;
R⁹³ is -L⁹²-R⁹⁶, wherein L⁹² is unsubstituted C₁₋₁₀ alkylene and R⁹⁶ is amido or substituted or unsubstituted phenyl; and
R⁹⁴ is C₁₋₁₀ alkyl, which C₁₋₁₀ alkyl is unsubstituted or substituted with a hydroxyl group.

62. A method according to claim 44 wherein the inhibitor of glycolipid biosynthesis is selected from the compounds of formula (V) listed in Table 3.

63. A method according to claim 44 wherein the compound is of formula (IX) and wherein r is 0; q is 1; R^{IXa} is H; R^{IXb} is unsubstituted C₁₋₆ alkyl; R^{IXc} and R^{IXd} are independently selected from H and unsubstituted C₁₋₆ alkyl; R^{IXe} and R^{IXf} are independently selected from H and unsubstituted C₁₋₆ alkyl; one of R^{IXg} and R^{IXh} is H and the other is OR^{IXr}, wherein R^{IXr} is selected from H and unsubstituted C₁₋₆ alkyl; R^{IXi} is unsubstituted C₁₋₆ alkyl; R^{IXj} is a group of formula (X); R^{IXk} is a group of formula (XI); R^{IXn}, R^{IXo}, R^{IXp} and R^{IXq}, which are the same or different, are independently selected from H and unsubstituted C₁₋₆ alkyl; and R^{IXm} is unsubstituted or substituted C₁₋₁₀ alkyl.

64. A method according to claim 44 wherein the compound is of formula (IX) and wherein r is 1, q is 0; R^{IXa} is COOH or an unsubstituted ester; R^{IXb} is C₁₋₆ alkyl substituted with a hydroxyl group; R^{IXc}, R^{IXd}, R^{IXe}, R^{IXf}, R^{IXj} and R^{IXk} which may be the same or different, are independently selected from H and unsubstituted C₁₋₆ alkyl; R^{IXg} and R^{IXh} together form an oxo group; R^{IXi} is H; and R^{IXm} is unsubstituted or substituted C₁₋₆ alkyl.

65. A method according to claim 44 wherein the compound is of formula (XII) and wherein R^{Xa}, R^{Xb} and R^{Xc}, which are the same or different, are independently selected from H, unsubstituted C₁₋₆ alkyl and unsubstituted phenyl.

66. A method according to claim 44 wherein the inhibitor of glycolipid biosynthesis is:
a compound of formula (IX), which compound is either Fumonisin or Myriocin; or a compound of formula (XII), which compound is L-cycloserine.

67. A method according to claim 38 wherein the inhibitor of glycolipid biosynthesis is RNA.

68. A method according to claim 66 wherein the RNA is antisense RNA or siRNA.

69. A method according to claim 38 wherein the glycolipid-mediated autoimmune disease is an autoimmune peripheral neuropathy; an autoimmune central neuropathy; a connective tissue disease; an autoimmune complication of a drug therapy; an autoimmune complication of a vaccine; a psycho-neuro-endocrinological autoimmune disease; an autoimmune vasculitide; an autoimmune thyroiditis; a non-vascular dementia; an autoimmune endocrinopathy; a late complication of an infective tick borne disease; an autoimmune arthritis; an autoimmune gastrointestinal disease; an autoimmune clotting disorder; a glomerulonephritides; autoimmune hemolytic anemia; autoimmune hepatitis; an ear disorder; or an autoimmune inner ear disease.

70. A method according to claim 38 wherein the glycolipid-mediated autoimmune disease is Guillain-Barré syndrome; a variant of Guillain-Barré syndrome; Guillain-Barré syndrome with opthalmoplegia; Miller Fisher syndrome; Acute motor axonal neuropathy; Motor neuropathy; Motor neuropathy with multifocal conduction blocks; Lower motor neuron syndromes; Chronic inflammatory demyelinating polyneuropathy; Multifocal chronic inflammatory demyelinating polyneuropathy; Acute inflammatory demyelinating polyneuropathy; Subacute inflammatory demyelinating polyneuropathy; Sensory neuropathies; Multifocal Motor Neuropathy; Multifocal motor sensory neuropathy; Acute Motor Sensory Axonal neuropathy; Multifocal motor demyelinating neuropathy; Chronic idiopainic sensory ataxic neuropathy; Chronic recurrent polyneuropathy; Mixed motor sensory neuropathy; Sciatica; Autoimmune mononeuritis multiplex; Acute relapsing sensory-dominant polyneuropathy associated with anti-GQ1b antibody; Amyotrophic lateral sclerosis; Diabetic neuropathy; Acute panautonomic neuropathy; Bell's palsy; Acute opthalmoparesis; Multiple sclerosis; Transverse myelitis; Optic neuritis; Chronic myelinic neuropathy with IgM gammopathy; Cryptogenic partial epilepsies; Partial oculomotor nerve palsy; Isolated cranial neuropathy; Autoimmune cerebellar disease; Acute Disseminated Encephalomyelitis; Stiff-man syndrome; Bickerstaff's brainstem encephalopathy; Systemic lupus erythamatosus; Discoid lupus; Scleroderma; Morphoea; CREST; Mixed connective tissue disease; Relapsing polychondritis; Sjogren's syndrome; Primary fibromyalgia syndrome; an autoimmune complication of drug therapy with Tumor necrosis factor-□ blocker, Interferon-□Tacrolimus (FK506), Cyclosporine A, Suramin, Zimeldine, Cisplatin, Captopril, Danazol, Gold, Penicillamine, Streptokinase or Anistreplase; an autoimmune complication of vaccination with Influenza Vaccination; an autoimmune complication of vaccination with Menactra meningococcal conjugate vaccine; Fibromyalgia syndrome; Chronic fatigue syndrome; Behçet's disease; Hashimoto's thyroiditis; Graves' disease; Alzheimer's disease; Insulin-dependent (type I) diabetes mellitus; Neuroborreliosis; Acute Disseminated Encephalomyelitis; Guillain-Barré disease; Rheumatoid arthritis; Still's disease; Coeliac disease; Crohn's disease; Ulcerative colitis; Primary adrenal failure; Pernicious anoemia; Idiopathic thrombocytopenic purpura; IgA Nephropathy; Meniere's disease; Autoimmune hemolytic anemia; Autoimmune hepatitis; Autoimmune inner ear disease or Acute opthalmoparesis.
